(19) United States
(12) Patent Application Publication
(10) Pub. No.: US 2006/0148104 A1
(43) Pub. Date: Jul. 6, 2006

(54) Detection of ion channel or receptor activity

(75) Inventors: Davide Marini, Brookline, MA (US); Bimal Desai, Arlington, MA (US); Markus Delling, Boston, MA (US); Daniel Solis, Boston, MA (US); Sebastien Febvay, Cambridge, MA (US); Brett Carter, Brighton, MA (US); Angela Belcher, Lexington, MA (US); David Clapham, Wellesley, MA (US)

Correspondence Address: CHOATE, HALL & STEWART LLP, TWO INTERNATIONAL PLACE, BOSTON, MA, 02110, US

(73) Assignees: Massachusetts Institute of Technology; Children's Medical Center Corporation

(21) Appl. No.: 11/264,074

(22) Filed: Oct. 31, 2005

Related U.S. Application Data
(60) Provisional application No. 60/623,334, filed on Oct. 29, 2004.

(30) Foreign Application Priority Data
Oct. 31, 2005 (WO) PCT/US05/39260

(51) Int. Cl. G01N 33/551

(52) U.S. Cl. 436/524

(57) ABSTRACT

The invention provides nanosensors and nanosensor components for the detection of ion channel activity, receptor activity, or protein protein interactions. Certain of the nanosensor components comprise a nanoparticle and recognition domain. Following contact with cells and, optionally, internalization of the nanosensor component by a cell, the recognition domain binds to a target domain, e.g., a heterologous target domain, of a polypeptide of interest such as an ion channel subunit, G protein coupled receptor (GPCR), or G protein subunit. Ion channel activity, GPCR activity, or altered protein interaction results in a detectable signal. The nanoparticles may be functionalized so that they respond to the presence of an ion by altering their proximity. Certain of the nanosensors utilize the phenomenon of plasmon resonance to produce a signal while others utilize magnetic properties, RET, and/or ion-sensitive moieties. Also provided are polypeptides, e.g., ion channel subunits, comprising a heterologous target domain, and cell lines that express the polypeptides. Further provided are a variety of methods for detecting ion channel activity, receptor activity, or protein interaction and for identifying compounds that modulate one or more of these. In certain embodiments the invention allows the user to detect the activity of specific ion channels even in the presence of other channels that permit passage of the same ion(s) or result in activation of the same downstream targets, thereby achieving improved specificity in high throughput screens while at the same time providing a high signal to noise ratio.

### CROSS-REFERENCE TO RELATED APPLICATION

This application claims priority to provisional application 60/623,334, filed Oct. 29, 2004, which is incorporated herein by reference.

### Government Support

The United States Government has provided grant support utilized in the development of the present invention. The United States Government may have certain rights in the invention.

### BACKGROUND OF THE INVENTION

Life of higher organisms such as humans requires rapid transmission of electric and chemical signals that synchronize a large number of diverse cellular processes. Electrical signals are vital for cellular functions and are mediated primarily by ion channels, a specialized group of proteins that span the membranes of all living cells and which are permeable to electrically charged atoms (ions) present in the body. Ion channels can be considered the biological equivalent of transistors in the sense that they respond to stimuli by switching between “on” (open) and “off” (closed) states, thereby controlling the passage of electric current across the cell membrane. In doing so, ion channels control the electrical state of cells and allow such fundamental processes as the beating of the heart, contraction of muscles and information processing in the nervous system.

In addition to their functions in regulating the electrical state of cells, calcium channels are of particular importance because calcium plays a key role as a second messenger in a wide range of signal transduction pathways, thereby regulating cellular responses to a diverse array of stimuli.

Due to the central role of ion channels in regulating such a vast array of physiological processes as well as the role of defective ion channels in various diseases such as arrhythmias, epilepsy, and cystic fibrosis, a considerable amount of effort has been devoted to elucidating their function and discovering compounds that modulate their function. Such efforts have led to the discovery of a number of pharmaceutical agents whose mechanism of action involves their ability to enhance or inhibit ion channel activity. Many therapeutic agents currently in widespread use act at least in part by modulating ion channel activity. Among them are anti-hypertensive agents, anti-arrhythmics, anti-epileptic agents, anesthetics, and others. The therapeutic potential of ion channels is further reflected by the fact that nearly a third of all drugs approved by the U.S. Food and Drug Administration (FDA) have been shown to act by direct or indirect modulation of ion channels. Given that only 1% of the ion channels encoded by the human genome are directly targeted by available drugs, the full potential of ion channels in medicine is yet to be harnessed. There is thus considerable interest in the study of ion channels and the identification of agents that modulate their activity.

The electrical activity of ion channels and their response to various stimuli can be recorded using the patch-clamp technique, which allows detection of the ion flow (an electric current) through the channel via a micro-pipette tip sealed to the cell membrane (1); the end result is a graph of current versus time or transmembrane voltage. This technique is currently one of the most important tools for investigating the activity of ion-channels. However, it is very difficult to master, requires patient and skilled personnel and is highly labor-intensive. Other approaches include binding assays with whole cell or membrane preparations, radioactive flux assays, and use of ion-sensitive or voltage-sensitive fluorescent dyes to monitor the overall increase in intracellular ion concentration or voltage change upon channel opening. However, these methods suffer from a variety of disadvantages. Development of technologies to more rapidly, reliably, and effectively screen for compounds acting on ion channels would dramatically increase our ability to intervene therapeutically in a host of conditions and disease processes.

G protein coupled receptors (GPCRs) represent another extremely important group of therapeutic targets. Indeed a large fraction of known therapeutic agents modulate the activity of one or more GPCRs. However, the full therapeutic potential of GPCR modulators remains unrealized, and there is a need in the art for improved screening methods in this area also.

### SUMMARY OF THE INVENTION

The present invention provides compositions and methods for detecting activity of a predetermined ion channel species of interest. The compositions include nanosensor components that are designed to attach to a predetermined ion channel subunit so that information regarding the activity of the specific ion channel containing that subunit can be obtained. The compositions and methods may be used for a variety of purposes including screening to identify agents that modulate ion channel activity. Most of the currently available platforms for high throughput screening of ion channels, such as those based on ion-sensitive or voltage-sensitive fluorescent probes, monitor the overall increase in intracellular ion concentration or voltage change upon channel opening. These methods share an inability to distinguish the signal created by the ion channel activity of interest from that of different origin such as release from intracellular stores or other entry pathways. This technical liability manifests itself in the form of large numbers of “false-positive hits”. In contrast, certain embodiments of the present invention measure the activity of specific ion channel types, allowing the rapid acquisition of reliable results with high information content.

The nanosensors of the present invention can be synthesized in large quantities and delivered to millions of cells in parallel. Moreover, in various embodiments, this technology requires only a small fraction of the labor necessary for patch-clamp studies. For these reasons, among others, the invention is extremely well suited for the high-throughput screening of compounds to identify those that target ion channels. Since such channels are directly or indirectly related to many debilitating and/or life-threatening conditions, the technology will reduce the time required for developing better cures. The modular and flexible nature of the technology allows it to be readily adapted to a variety of applications. For example, the nanosensors can be used to detect GPCR activity and protein-protein interactions.

In one aspect, the invention provides a nanosensor component comprising: a nanoparticle having a moiety comprising a recognition domain attached to the nanoparticle, wherein the recognition domain is selected to specifically bind to a target domain of a cellular polypeptide. In certain embodiments of the invention the recognition domain comprises an artificial polypeptide. In certain embodiments of the invention (i) the recognition domain comprises a coiled-coil peptide, an enzyme, or an enzyme inhibitor; or (ii) the nanoparticle comprises an ion-sensitive fluorescent or luminescent moiety or comprises a resonance energy transfer (RET) donor having an emission spectrum capable of exciting an ion-sensitive RET donor; or (iii) the nanoparticle comprises a coating layer comprising a material that undergoes a change in refractive index in the presence of a ligand; or (iv) any combination of (i)-(iii). In certain embodiments of the invention the cellular polypeptide is a recombinant polypeptide comprising a heterologous target domain. In certain embodiments of the invention the cellular polypeptide is an ion channel subunit, a GPCR, or a G protein subunit. The nanoparticle may be, for example, a metal particle, e.g., a plasmon resonant particle or a magnetic particle. In certain embodiments of the invention the nanoparticle comprises an ion-sensitive binding moiety, an ion-sensitive signal-generating moiety such as a fluorescent or luminescent dye or a resonance energy transfer (RET) donor, which may be a quantum dot.

In another aspect, the invention provides a polypeptide comprising a heterologous recognition domain. The recognition domain may be selected from the group consisting of: heterologous coiled-coil domains, enzymes, and enzyme inhibitors. The polypeptide may be selected from the group consisting of ion channel subunits (e.g., calcium channel subunits), G protein subunits, and G protein coupled receptors (GPCRs).

In another aspect, the invention provides a method of introducing a nanoparticle into a cell comprising steps of: (i) providing a nanoparticle having a delivery moiety attached thereto; (ii) providing a cell; and (iii) applying an electric or magnetic field to the cell in the presence of the nanoparticle. The delivery moiety may be, e.g., a peptide such as a TAT peptide.

In another aspect, the invention provides a method of detecting ion channel or GPCR activity or lack thereof comprising steps of: (i) providing a cell comprising a polypeptide having a nanosensor component attached thereto, wherein the polypeptide is an ion channel subunit or GPCR, and wherein the nanosensor component comprises a nanoparticle; (ii) maintaining the cell under conditions in which ion channel or GPCR activity may occur; and (iii) detecting a signal that is indicative of ion channel or GPCR activity or lack thereof. The signal may be, e.g., an optical or magnetic signal, a plasmon resonance property, a fluorescent or luminescent signal, or a RET signal. In certain embodiments of the invention the nanosensor component comprises a nanoparticle having a recognition domain attached thereto, e.g., a coiled-coil peptide, an enzyme, an enzyme inhibitor. In certain embodiments of the invention the recognition domain compises an artificial polypeptide. The nanoparticle may be, for example, a metal particle, e.g., a plasmon resonant particle or a magnetic particle. In certain embodiments of the invention the nanoparticle comprises an ion-sensitive binding moiety, an ion-sensitive signal-generating moiety such as a fluorescent or luminescent dye or a resonance energy transfer (RET) donor, which may be a quantum dot.

The invention further provides a method of testing a compound comprising steps of: (i) providing a cell comprising an ion channel or GPCR having a nanosensor component attached thereto, wherein the nanosensor component comprises a nanoparticle; (ii) contacting the cell with the compound; (iii) gathering a signal indicative of ion channel or GPCR activity or lack thereof; and (iv) determining whether the compound is a modulator of ion channel or GPCR activity based on the information gathered in step (iii).

The invention further provides a method of detecting a modulator of ion channel or GPCR activity comprising steps of: (i) providing an ion channel or GPCR having a nanosensor component attached thereto, wherein the nanosensor component comprises a nanoparticle; (ii) maintaining the ion channel or GPCR under conditions in which exposure to a modulator of ion channel or GPCR activity may occur; (iii) gathering a signal indicative of ion channel or GPCR activity or lack thereof from the nanoscale sensor; and (iv) determining whether a modulator of ion channel or GPCR activity is present based on the signal gathered in step (iii).

The invention further provides a method of attaching a nanoparticle to a cellular polypeptide of interest comprising steps of: (i) providing a cell that expresses a polypeptide of interest comprising a target domain; (ii) contacting the cell with a nanoparticle comprising a recognition domain that corresponds to the target domain under conditions in which internalization of the nanoparticle occurs; (iii) maintaining the cell so that the recognition domain and the target domain bind to one another within the cell.

The invention further provides a method of delivering an agent to a cell comprising steps of: (i) attaching a nanoparticle to a cellular polypeptide of interest according to the foregoing method, wherein the nanoparticle comprises the agent; (ii) maintaining the cell under conditions and for a time sufficient to allow release of the agent from the nanoparticle.

The invention further provides a method of detecting ion channel activity comprising steps of: (i) providing a cell comprising at least one ion channel having a nanoparticle attached thereto, wherein the nanoparticle comprises an ion-sensitive signal-generating moiety; and (ii) detecting a signal indicative of ion channel activity. The signal-generating moiety may, for example, be an ion-sensitive fluorescent or luminescent molecule.

The invention also provides a method of detecting ion channel activity comprising steps of: (i) providing a cell comprising at least one ion channel having a nanoparticle attached thereto, wherein the nanoparticle comprises a RET donor and the cell contains an ion-sensitive RET acceptor, wherein the RET donor and RET acceptor are a RET pair; and (ii) detecting RET between the donor and acceptor, wherein RET is indicative of ion channel activity. In certain embodiments of the invention the RET donor, RET acceptor, or both, comprises a quantum dot or a fluorescent or luminescent molecule.

The invention also includes a method of detecting ion channel activity comprising steps of: (i) providing a cell comprising at least one ion channel having a nanoparticle attached thereto, wherein the nanoparticle comprises an ion-responsive coating layer comprising a compound that changes refractive index in response to an ion; and (ii) detecting an alteration in a plasmon resonance property of the nanoparticle, wherein the alteration is indicative of ion channel activity.

The invention further includes a method of detecting ion channel activity, comprising steps of: (i) providing a cell comprising at least one ion channel having at least two nanoparticles attached thereto; and (ii) detecting an alteration in the distance between the nanoparticles, wherein the alteration in distance is indicative of ion channel activity.

The invention further features a method of detecting a protein protein interaction or an alteration in a protein protein interaction comprising steps of: (i) providing a cell, wherein the cell comprises a first polypeptide having a nanoparticle attached thereto and a second polypeptide having a nanoparticle attached thereto; (ii) detecting an alteration in the distance between the nanoparticles, wherein the alteration in distance between the nanoparticles is indicative of an alteration in the distance between the polypeptides. The polypeptides may be, e.g., a GPCR and a G protein subunit, two G protein subunits, etc.

Unless otherwise stated, the invention makes use of standard methods of molecular biology, cell culture, animal maintenance, etc., and uses art-accepted meanings of terms. This application refers to various patents and publications. The contents of all of these are incorporated by reference. In addition, the following publications are incorporated herein by reference: Ausubel, F., (ed.) Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning: A Laboratory Manual, 3^{rd} ed., Cold Spring Harbor Laboratory Press, Cold Spring Harbor, 2001; Kuby Immunology, 4^{th} ed., Goldsby, R. A., Kindt, T. J., and Osborne, B. (eds.), W.H. Freeman, 2000, Goodman and Gilman's The Pharmacological Basis of Therapeutics, 10^{th} Ed. McGraw Hill, 2001, and Katzung, B. (ed.) Basic and Clinical Pharmacology, McGraw-Hill/Appleton & Lange; 8th edition (Sep. 21, 2000), and Feldheim, D L and Foss, C A (eds.) Metal nanoparticles: synthesis, characterization, and applications, Marcel Dekker, 2002. In the event of a conflict between any of the incorporated references and the instant specification, the specification shall control. The determination of whether a conflict or inconsistency exists is within the discretion of the inventors and can be made at any time.

### BRIEF DESCRIPTION OF THE DRAWING

FIG. 1 shows a schematic diagram of a nanosensor component of the invention.

FIGS. 2A and 2B show a schematic diagram of a nanosensor component designed to become attached to an ion channel that spans the plasma membrane of a cell.

FIG. 3 is a schematic representation showing detection of calcium channel activity using a nanosensor component attached to a subunit of a calcium channel. Upon channel opening a sharp increase in ion concentration occurs in the vicinity of the channel. Lines emanating from the nanoparticle indicate that calcium entry results in production of a detectable optical or magnetic signal.

FIG. 4A is a schematic representation of a coiled-coil recognition motif (48). The diagram on the left symbolizes an α-helix whose amino acid sequence is characterized by a heptad repeat. Two such α-helices wrap aound each other, allowing the specific binding of polypeptides containing the two helices.

FIG. 4B is a ribbon diagram of the barnase-barstar complex.

FIG. 5A is a schematic representation of the binding of an engineered ion channel subunit and a nanoparticle via recognition and target domains that interact to form a coiled-coil. A recognition domain containing multiple heptad repeat units is conjugated to the nanoparticle. The ion channel subunit contains a complementary target domain that includes multiple copies of a heptad repeat having a different sequence from that of the recognition domain. In this illustration the target domain is fused to the N-terminus of the ion channel subunit of interest. A heptad repeat peptide is conjugated to the nanoparticle, while a complementary target peptide (also a heptad repeat) is fused to the N-terminal portion of an ion channel subunit of interest. The C-terminus of the ion channel subunit, which is intracellular, is also shown. After translocation of the nanoparticle into the cytosol, it is carried to the engineered ion channel subunit by Brownian diffusion and binds to it.

FIG. 5B is a schematic representation showing a recognition design based on the barnase-barstar complex.

FIGS. 6A-6C present fluorescence microscopy images showing expression of an engineered TRPV3 ion channel subunit in HEK cells following transient transfection of either (A) TRPV3-EGFP-cc, (B) TRPV3-EGFP-barstar, or (C) TRPV3-EGFP constructs. Note that the order of the elements in the polypeptides encoded by these constructs in an N- to C-direction is actually cc-EGFP-TRPV3 (A), barstar-EGFP-TRPV3 (B), EGFP-TRPV3.

FIGS. 7A and 7B shows whole cell patch clamp recordings taken from HEK-293T cells transiently transfected with constructs encoding recombinant TRPV3 ion channel subunits TRVP3-EGFP-cc and TRPV-EGFP FIG. 7A shows I/V relationships before and after application of 100 μM 2APB to cells expressing TRPV3-EGFP-cc (left) and cells expressing TRPV3-EGFP (right). FIG. 7B shows a timecourse of application of 100 μM 2APB to cells expressing TRPV3-EGFP-cc (left) and TRPV3-EGFP (right). FIG. 8 presents a Western blot analysis showing the ability of the coiled-coil interaction motif to mediate specific binding of a nanoparticle to a polypeptide of interest. A construct encoding EGFP fused to the coiled-coil peptide (EVSALEK)₄ (SEQ ID NO: 4) was generated. The complementary coiled-coil peptide, (KVSALKE)₃, (SEQ ID NO: 3) was covalently attached to silica nanoparticles via chemical crosslinking. For simplicity both peptides, although distinct in sequence, are labeled “cc” (for coiled-coil). Cells were transfected to express EGFP or EGFP-cc. Nanoparticles (either unfunctionalized (NP) or functionalized (NP-cc) were then incubated with cell lysates to allow binding to occur and were recovered from solution. The left image shows Western blot analysis of EGFP and EGFP-cc, as indicated. The right image shows Western blot analysis of pellets containing NP or NP-cc that had been incubated in lysates from cells expressing EGFP or EGFP-cc, as indicated. Notice that EGFP-cc is heavier than EGFP and that only NP-cc can pull down EGFP-cc.

FIG. 9 shows another Western blot in which lysates from cells expressing either EGFP-cc or TRPV3-EGFP-cc or proteins recovered from nanoparticles that had been incubated in such lysates were probed with an antibody that recognizes EGFP.

FIGS. 10A and 10B show dark-field microscopy images of nanoparticle internalization within live cells. (A) HEK cells were incubated for 5 min with TAT-conjugated gold nanoparticles (bright spots) with no electroporation. (B) Same as in panel A, except that cells were subjected to electroporation in the early stage of incubation.

FIG. 11 shows electron microscopy images of cells containing internalized nanoparticles. Gold nanoparticles (15 nm diameter) were either electroporated (left) or endocytosed (right) by COS cells.

FIGS. 12A-12C show schematic representations of various aspects of plasmon resonance. FIG. 12A shows a schematic representation of the scattering spectra of silver nanoparticles. The peak scattering wavelength of silver nanoparticles increases with diameter, allowing particles of different size to be easily distinguishable under dark-field microscopy. Small particles appear blue, while larger ones appear red (adapted from 26). FIG. 12B shows a schematic representation of plasmon resonance coupling between two nanoparticles (23). FIG. 12C is a schematic representation showing the sensitivity of the peak plasmon resonance to the refractive index of their surrounding medium (adapted from 82).

FIG. 13 shows a schematic representation of nanoparticle aggregation in the presence of calcium ions. Gold nanoparticles are coated with a monolayer of molecules carrying calcium-binding moieties such as ionized carboxyl groups. When calcium is present, such groups chelate the ion in between them, overcoming their electrostatic repulsion and inducing aggregation. Exact details of the coating molecules are not represented here; the drawing is not to scale.

FIG. 14 shows the absorption spectrum of a solution of 15 nm plasmon resonant gold nanoparticles (red line) and its change when particles are induced to aggregate (blue line). The plasmon resonance frequency is at the peak of each curve.

FIG. 15A shows a nanosensor component of the invention with a calcium-sensitive coating and an attached recognition domain.

FIG. 15B is a schematic representation of detection of ion channel activity using nanosensor components that utilize the phenomenon of plasmon resonance. A plasmon resonant, calcium-sensitive gold nanoparticle is attached to each of two ion channel subunits. Upon channel opening, a sudden influx of calcium induces aggregation of the calcium-sensitive nanoparticles, changing their peak plasmon resonance, their color under dark-field microscopy, and their spectra. The design can also be used for detection of ion channel activity based on proximity-dependent properties of magnetic nanoparticles.

FIGS. 16A and 16B shows results of experiments demonstrating the sensitivity and specificity of peptide-functionalized nanosensors to calcium ions. Gold nanoparticles were coated with a peptide displaying asparagine groups to the environment. FIG. 16A shows the absorption spectrum of peptide-modified nanoparticles in the presence of various concentrations of calcium. The particles selectively chelate calcium ions, an event leading to their aggregation at a calcium concentration of 1 mM (FIG. 16B, left tube), which is readily detected by a spectral change. When particles are exposed to the same concentration of Mg⁺⁺ (1 mM, FIG. 16B, right tube), they remain in solution and the absorption spectrum does not change (the image was obtained 24 h after mixing the particles with the respective ions).

FIG. 17 shows a schematic representation of a second design for detecting ion channel activity based on plasmon resonance. A single gold nanoparticle is attached to the ion channel. The cell cytosol is loaded with many small gold particles (e.g., particles small enough to be undetectable under dark-field microscopy). Upon channel opening, the calcium flux induces aggregation of the small particles onto the large one, inducing a shift in its peak plasmon resonance and its color under dark-field microscopy. The design can also be used for detection of ion channel activity based on proximity-dependent properties of magnetic nanoparticles.

FIG. 18A is a schematic representation of a polyanion.

FIG. 18B is a schematic representation of the design of an optical calcium sensor based on the conformational change of a coating layer, e.g., polyacrylic acid (PAA), in the presence of calcium. A coating of polyacrylic acid containing large coils collapses in the presence of calcium, inducing a change in the local refractive index of the surrounding medium, and thus a shift in the peak plasmon resonance of a metal nanoshell.

FIGS. 19A and 19B show the effect of calcium ions on the conformation of PAA. Panel A shows a 1% aqueous solution of polyacrylic acid, along with a schematic of its random coil conformation. Dropwise addition of a 10 mM aqueous solution of CaCl₂ induces immediate formation of a white precipitate (Panel B).

FIG. 20 shows absorbance spectra of 15 nm gold particles coated with a layer of polyacrylic acid and exposed to 2 mM calcium in TBS buffer. Exposure to calcium causes the peak plasmon resonance frequency to red-shift by circa 5 nm; reversibility is demonstrated by addition of the calcium chelator EDTA. Curves are not normalized for variability in particle concentration.

FIG. 21 is a schematic representation of a FRET-based design for the detection of calcium channel activity. A fluorescent nanoparticle (FRET donor) is attached to a calcium channel of interest and the cell is loaded with a calcium-sensitive dye (FRET acceptor) whose absorption spectrum overlaps with the emission spectrum from the nanoparticle. When the cell is illuminated at the excitation frequency of the FRET donor, FRET occurs between the nanoparticle and the surrounding dye molecules, which emit light in the presence of calcium. Since the dye cannot be excited at the excitation frequency used to excite the FRET donor, a signal at the peak of the emission spectrum of the dye indicates calcium channel activity. The design can also be used for detection of ion channel activity based on proximity-dependent properties of magnetic nanoparticles.

FIG. 22 is a schematic representation of another method for detecting ion channel activity in vivo using FRET. The FRET donor and acceptor are Au nanoclusters. An ion channel is engineered with two distinct target domains: e.g., barstar and a coiled-coil peptide, both present on a heterologous stretch of polypeptide chain that extends from the N- or C-terminus of the ion channel subunit.

FIG. 23 shows a demonstration of FRET-based detection of calcium in living cells. Nanoparticles (absorption ˜405 nm, emission ˜580 nm) were internalized by COS cells that had been previously loaded with a calcium-sensitive dye (absorption ˜580 nm, emission ˜620 nm). Cells were illuminated with a 405 nm laser and emission from the sample was collected through a narrow bandgap filter centered around 620 nm. Upon addition of thapsigargin (a compound that induces calcium entrance into the cytosol from internal stores), flashes of light (orange dots) were detected only in the immediate vicinity of the nanoparticles. The image is a snapshot from a movie taken at mid-plane section of a cell via confocal microscopy.

FIG. 24 is a schematic of mesoporous silica nanoparticles synthesized as described (45). Each nanosphere is composed of nanoporous silica, which comprises an ordered array of cylindrical pores. The wall of such pores present thiol groups to the environment.

### Definitions

The term “antibody” refers to an immunoglobulin or an antigen-binding portion thereof. The term includes polyclonal or monoclonal antibodies, antibody fragments such as Fab′, F(ab′)₂, scFv (single-chain variable), etc.

The term “approximately” in reference to a number includes numbers that fall within a range of 5% in either direction (greater than or less than) of the number unless otherwise stated or otherwise evident from the context (except where such number would exceed 100% of a possible value).

The terms “attached” and “attachment” are used interchangeably herein with the terms “linked” and “linkage” to refer to moieties that are covalently or noncovalently physically associated and/or joined to one another, either directly or indirectly. Moieties that are attached to one another may also be referred to as “conjugated”, and a composition comprising two attached moieties may be referred to as a “conjugate”.

“Biocompatible” refers to a material that is substantially non-toxic to cells in vitro, e.g., if its addition to cells in culture results in less than or equal to 20% cell death.

A “cellular polypeptide” refers to a polypeptide that is expressed by a cell and is present within a cell or at a cell surface, whether naturally encoded by the genome of the cell or not. The term includes intracellular polypeptides, transmembrane polypeptides, polypeptides synthesized by the cell and attached to the plasma membrane, etc.

The term “coating”or “coating layer” can refer to a layer or film of material around an interior core. The layer can be continuous or can have a configuration such as a “mesh” or “cloud”, e.g., of polymer chains attached to and surrounding an interior core.

An “engineered” cell is one that is manipulated to express or contain a polypeptide that it does not naturally express or contain, or a cell that is descended from an ancestor cell that was so manipulated. An “engineered” polypeptide is used herein interchangeably with “recombinant” polypeptide. An “engineered” protein is a protein that contains at least one engineered subunit.

An “expression control sequence” refers to a nucleotide sequence in a polynucleotide that regulates the expression (transcription and/or translation) of a nucleotide sequence operably linked thereto. Examples include promoters, enhancers, initiation signals, and other transcriptional control elements that induce or control transcription of coding sequences with which they are operably linked.

A “heterologous” or “non-native” polypeptide is one that is not naturally produced by a cell of interest or by the ancestor of the cell, in the absence of intervention by the hand of man.

A “heterologous” or “non-native” portion of a polypeptide is a polypeptide segment that is not naturally found in the polypeptide in the absence of intervention by the hand of man.

“Identity” refers to the extent to which the sequence of two or more nucleic acids or polypeptides is the same. The percent identity between a sequence of interest and a second sequence may be computed by aligning the sequences, determining the number of residues (nucleotides or amino acids) that are opposite an identical residue allowing the introduction of gaps to maximize identity, dividing by the total number of residues of the sequence of interest or the second sequence (whichever is greater), and multiplying by 100. Percent identity can be calculated with the use of a variety of computer programs known in the art. For example, computer programs such as BLAST2, BLASTN, BLASTP, Gapped BLAST, etc. (which employ the well-known algorithsm of Karlin and Altschul), and sequence analysis programs available in the GCG Wisconsin Package (now called Accelrys GCG) generate alignments and provide percent identity. Default parameters can be used. In a specific embodiment, percent identity of a sequence of interest and a second sequence is calculated using BLAST2 with default parameters.

An “ion” is an atom or group of atoms having a positive or negative charge.

An “ion channel” is a protein structure containing a pore that allows the passage of ions across a cell membrane.

The term “nanoparticle” refers to a particle having a longest straight dimension of 1 μm or less, typically 500 nm or less, more typically 200 nm or less. Particles of use in this invention can have a range of shapes and cross-sections including spheres, oblate spheroids, cylinders, shells, cubes, pyramids, hexagons, tetrapods (particles having four leg-like appendages), triangles, prisms, ellipses, rods (e.g., cylinders or elongated structures having a square or rectangular cross-section), etc. Spherical nanoparticles of interest herein generally have a diameter of 200 nm or less, preferably 100 nm or less, and often 50 nm or less. The term “nanoparticle” encompasses atomic clusters, which have a typical diameter of 1 nm or less and generally contain from several (e.g., 3-4) up to several hundred atoms.

The terms “nucleic acid” and “polynucleotide” are used interchangeably herein to refer to a single or double-stranded polymer of nucleotides. The term includes the naturally occurring nucleic acids DNA and RNA. One of ordinary skill in the art will appreciate that nucleic acids can comprise various analogs of the nucleotides found in naturally occurring nucleic acids and that a nucleic acid can contain a variety of modifications to the sugar or backbone structure. The term “nucleic acid sequence” as used herein can refer to the nucleic acid material itself and is not restricted to the sequence information (e.g., the succession of letters chosen among the five base letters A, G, C, T, or U) that biochemically characterizes a specific nucleic acid.

As used herein, “operatively linked” refers to a relationship between two nucleic acid sequences wherein the expression of one of the nucleic acid sequences is controlled by, regulated by, directed by, etc., the other nucleic acid. For example, the transcription of a nucleic acid sequences is under control of an operably linked promoter sequence.

The term “particle” is used herein as generally understood in the art, i.e., to refer to any small piece or fragment of material. A particle can be homogenous or nonhomogeneous in composition. A particle may consist of multiple smaller particles embedded in a matrix.

A “plurality” means more than one.

“Polypeptide”, as used herein, is a chain of amino acids. A peptide is a short polypeptide, typically containing between 2 and 100 amino acids. The term “polypeptide” includes “peptides”. The individual polypeptides of which a protein is composed are referred to as “subunits”. A protein is a molecule composed of one or more polypeptides. Polypeptides can be modified at one or more positions, e.g., by a moiety such as a phosphate group, carbohydrate, lipid, phospholipid, glycolipid, etc. The terms “polypeptide sequence”, “peptide sequence”, or “amino acid sequence” as used herein can refer to the polypeptide material itself and is not restricted to the sequence information (i.e. the succession of letters or three letter codes chosen among the letters and codes used as abbreviations for amino acid names) that biochemically characterizes a polypeptide. A polypeptide sequence presented herein is presented in an N-terminal to C-terminal direction unless otherwise indicated. Standard amino acid abbreviations are used hereinA “proximity-dependent” signal or property is a signal or property that depends at least in part on the distance between two moieties and/or varies at least in part based on the distance between two moieties. Examples include plasmon resonance properties of nanoparticles and resonance energy transfer. Moieties are “in proximity” or “in close proximity” if they are close to one another.

A protein “subunit” is a single polypeptide that assembles (or coassembles) with other polypeptides to form a protein. Examples of proteins composed of multiple subunits include G proteins and many ion channels. The subunits of a protein may be identical, homologous, or dissimilar. A protein that consists of only a single polypeptide chain is considered to consist of a single subunit.

The term “recombinant polypeptide” refers to a polypeptide having polypeptide portions (amino acid sequences) that are not naturally joined together. Thus a recombinant polypeptide has an overall amino acid sequence that does not exist in nature. A recombinant polypeptide can consist entirely of two or more polypeptide portions whose sequences are found in nature, (e.g., within different naturally occurring polypeptides), can comprise or consist of one or more portions whose sequence is not found in any naturally occurring polypeptide (e.g., having a sequence entirely invented by man), can contain both naturally occurring and non-naturally occurring amino acid sequences, etc.

“Small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds.

“Specific binding” refers to noncovalent physical association of a first molecule or molecular complex and a second molecule or molecular complex wherein the first molecule or molecular complex does not substantially physically associate with most or all other molecules or molecular complexes present in the environment in which binding occurs. Binding of two or more entities may be considered specific if the equilibrium dissociation constant, K_{d}, is 10⁻⁴ M or less, more preferably 10⁻⁵ M or less, e.g., 10⁻⁶ M or less, 10⁻⁷ M or less, 10⁻⁸ M or less, or 10⁻⁹ M or less under the conditions employed, e.g., under physiological conditions such as those inside a cell or consistent with cell survival. Physiological conditions typically are average conditions of pH, temperature, and osmolarity that exist in the cell cytosol, extracellular fluid, and/or in standard cell culture medium. Exemplary physiological conditions for mammalian cells are typically a pH of 7.0-7.5, a temperature of 36° C.-38° C., and an osmolarity of 290-310 mOsm. Alternately, binding affinity may be expressed in terms of association constant, which is the reciprocal of the dissociation constant, wherein binding of two or more entities may be considered specific if the association constant, Kₐ, is 10⁴ M or greater, more preferably 10⁵ M or greater e.g., 10⁶ M or greater, 10⁷ M or greater, 10⁸ M or greater or 10⁹ M or greater under the conditions employed.

A “vector” is used herein to refer to a nucleic acid or a virus or portion thereof (e.g., a viral capsid) capable of mediating entry of, e.g., transferring, transporting, etc., a nucleic acid molecule into a cell. The nucleic acid molecule to be transferred is generally linked to, e.g., inserted into, the vector. Vectors of utility in recombinant DNA techniques are often in the form of “plasmids” which refer to circular double stranded DNA loops which, in their vector form are not bound to the chromosome. Vectors derived from viruses, such as retroviruses, adenoviruses, and the like, may be employed, as may such other forms of expression vectors which serve equivalent functions and which become subsequently known in the art. The vector can contain regulatory elements controlling transcription of an operatively linked nucleic acid sequence. The choice of promoter and other regulatory elements generally varies according to the intended host cell. Such elements generally be derived from eukaryotic (e.g., mammalian), prokaryotic, and/or viral or genes. The ability to replicate in a host, usually conferred by an origin of replication, and a selection gene to facilitate recognition of cells that have taken up the expression vector and in which expression occurs, may additionally be present.

### DETAILED DESCRIPTION OF CERTAIN PREFERRED EMBODIMENTS OF THE INVENTION

I. Overview

The present invention provides compositions and methods for the detection of protein activity and/or physical interactions between proteins inside living cells, or at their surfaces. Detection of protein activity and/or physical interaction is achieved by use of nanoscale sensors, which can comprise a variety of different nanosensor components. In accordance with the invention at least one nanosensor component is attached to a predetermined polypeptide of interest by means of a recognition domain that specifically binds to a target domain of the polypeptide. A number of polypeptides of interest herein are subunits that assemble with one or more other subunits to form a larger protein. In various embodiments of the invention the polypeptide of interest is an ion channel subunit, a G protein subunit, or a G protein coupled receptor (GPCR).

The nanoscale sensor is used to detect activity or physical interaction of the protein to which one or more nanosensor components is attached. Thus the invention allows specific detection of the activity of a cellular polypeptide of interest even in the presence of other polypeptides having the same or similar activities. For example, the invention allows the detection of the activity of an ion channel comprising a specific subunit, even in the presence of other ion channels that allow passage of the same ion. Similarly, the invention provides the ability to detect the activity of a specific receptor, e.g. a G protein coupled receptor, even in the presence of other receptors that physically interact with the same downstream targets.

A nanoscale sensor of the present invention is a sensing device whose dimensions may conveniently be measured in terms of nanometers, or whose individual parts (referred to herein as “components”) have such dimensions. Typical dimensions range from approximately 1 μm down to single atoms. The relevant dimension may be, e.g., length, width, depth, breadth, height, radius, or diameter, in the case of devices or components that have a regular two or three-dimensional shape such as solid or hollow spheres, cylinders, cubes, hexagons, triangles, pyramids, etc., or any other relevant dimensions (e.g., longest straight dimension, by which is meant the length of the line joining those two points on the particle that are located furthest from each other). Nanodevices and other nanostructures made of a given material or materials typically display characteristic differences in various physical and chemical properties relative to the bulk material(s) as a result of their small size. Certain embodiments of the present invention make use of a number of these size-dependent properties. Operation of the sensor may involve interaction of one or more nanosensor components with each other and/or with one or more moieties inside the cell such as a fluorescent or luminescent molecule, a quantum dot (QD), etc.

The invention provides a nanosensor component comprising a nanoparticle and a recognition domain, wherein the recognition domain is selected to specifically bind to a target domain of a predetermined polypeptide of interest. In one embodiment the polypeptide is an ion channel subunit, and the nanoparticle sensor component is adapted for sensing the presence of an ion. “Adapted for sensing the presence of an ion” means that the nanoparticle sensor component either produces a detectable optical or magnetic signal in the presence of an appropriate concentration of the ion or that the nanoparticle sensor component operates together with one or more additional moieties such as additional nanosensor component(s), fluorescent or luminescent molecules, or other signal-generating moieties, resulting in production of a detectable optical or magnetic signal in response to the presence of the ion and/or a change in ion concentration. The additional nanosensor component can be, e.g., a second nanosensor component comprising a nanoparticle and a recognition domain; one or more nanoparticles that do not comprise a recognition domain, etc.

In certain embodiments of the invention passage of an ion through an ion channel alters the proximity of two or more nanoparticles, resulting in a proximity-dependent signal. In certain embodiments of the invention passage of an ion through the channel alters the local environment of a nanoparticle, resulting in an alteration in an optical property of the nanoparticle. In certain embodiments of the invention an ion-sensitive signal-generating moiety located in close proximity to the nanoparticle produces a signal as a result of passage of an ion through the channel and excitation by a moiety located in or attached to the nanoparticle.

The invention further provides a nanosensor component comprising a nanoparticle and a recognition domain, wherein the recognition domain is selected to specifically bind to a target domain of a first predetermined polypeptide of interest, and wherein the nanoparticle sensor component is adapted for sensing a physical interaction between the first predetermined polypeptide and a second predetermined polypeptide. “Adapted for sensing a physical interaction” means that the nanoparticle sensor component, optionally together with one or more additional component(s), operates to produce a detectable optical or magnetic signal when the first and second predetermined polypeptides physically interact or cease to physically interact. The additional component can be, e.g., a second nanosensor component comprising a nanoparticle and a recognition domain that is selected to bind to the second predetermined polypeptide. In certain embodiments of the invention the first polypeptide is a GPCR, and the second polypeptide is a G protein subunit. As further described below, activity of a GPCR results in an alteration in its physical association with one or more G protein subunits. Thus detecting an alteration in the physical association of a GPCR and one or more G protein subunits constitutes detecting activity of the GPCR.

In certain embodiments of the invention physical interaction alters the proximity of two or more nanoparticles, resulting in a proximity-dependent signal. In certain embodiments of the invention a signal-generating moiety attached to a first polypeptide produces a signal as a result of excitation by a moiety located in or attached to a nanoparticle that is attached to a second polypeptide upon an increase or decrease in the distance between the two polypeptides.

The recognition domain of a nanosensor component of the invention specifically binds to a predetermined polypeptide such as an ion channel subunit, G protein subunit, or GPCR. The predetermined polypeptide contains a target domain to which the recognition domain specifically binds. The target domain can be either a heterologous target domain or a native target domain. In certain embodiments of the invention the target domain is a polypeptide segment that is not normally found in the polypeptide of interest.

In some embodiments of the invention the nanosensor component further comprises a delivery moiety and/or a biocompatible coating.

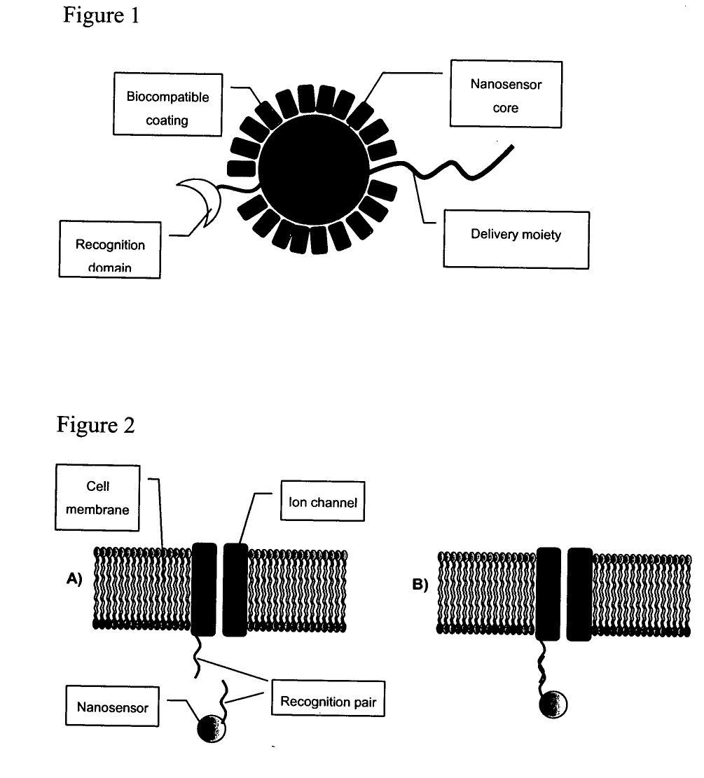

FIG. 1 shows an embodiment of a nanosensor component of the invention. As shown therein, the nanosensor component comprises a nanoparticle, a recognition domain attached to the nanoparticle and selected to bind to a specific polypeptide of interest, a delivery moiety attached to the nanoparticle, and a biocompatible coating, which is optional. The nanoparticle may comprise a core and, optionally, one or more coating layers surrounding the core. The recognition domain may be attached to the nanoparticle core or to the coating layer. Nanoparticles having a variety of different characteristics and made of a variety of different materials are used in different embodiments of the invention, as discussed further below.

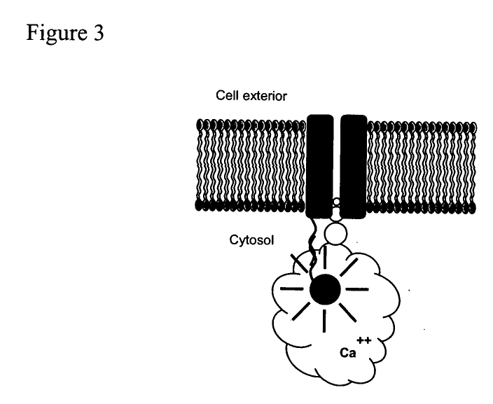

FIG. 2 shows a schematic diagram of a nanosensor component attached to a subunit of an ion channel that spans the plasma membrane of a cell. In FIG. 2A a nanosensor component comprising an attached recognition domain has been delivered to the cytosol of a cell that expresses an ion channel subunit containing a target domain to which the recognition domain specifically binds. The recognition and target domains form a recognition pair. In FIG. 2B the nanosensor component is attached to a selected ion channel subunit that comprises the corresponding target domain via the interaction between the members of the recognition pair. The cell may be genetically engineered to achieve expression of a recombinant polypeptide containing the target domain. In some embodiments of the invention the target domain is located in an extracellular domain of an ion channel subunit and the nanosensor component is delivered to the exterior of the cell.

The invention includes embodiments in which one or more nanosensor component(s) is/are attached to a single subunit of an ion channel and embodiments in which one or more nanosensor components are attached to each of two or more subunits of an ion channel. For example, one or more nanoscale sensor components can be attached to each of two, three, four, or more subunits of an ion channel. The subunits may be identical or different and may contain identical or different target domains.

Multiple nanosensor components can be attached to a single polypeptide of interest that includes two identical or different target domains. For example, two nanosensor components can be attached to a single ion channel subunit.

A nanosensor component can be attached to any polypeptide of interest by appropriate selection of a recognition domain and target domain as depicted in FIG. 2. The polypeptide of interest may be located entirely within the cell, may span the plasma membrane, or may be located extracellularly and attached to the plasma membrane. The target domain should be located in an accessible region of the protein, which is typically one exposed to the cytosol or extracellular environment. It may be desirable to use a target domain that is not located in the transmembrane portion of a protein. For example, in the case of an ion channel, it may be desirable to avoid employing a target domain located in the portion of the ion channel subunit that contributes to pore formation. In general, locations at or near the N- and/or C-termini of a polypeptide are suitable locations for a target domain.

Preferably a heterologous target domain does not interfere with the ability of an ion channel subunit to assume the correct structure and membrane topology and/or assemble with other subunit(s) to form a functional ion channel. If desired, the ability of a recombinant ion channel subunit to assume the correct structure and membrane topology and/or to assemble with other subunits to form a functional ion channel may be tested and/or compared with that of a wild type subunit, e.g., using patch-clamping techniques. Similarly, in preferred embodiments of the invention the target domain does not interfere with the ability of a GPCR or G protein subunit to assume the correct structure and membrane topology and/or to interact with other G protein subunits to form a functional GPCR or G protein, respectively. If desired, the functional characteristics of a recombinant GPCR or G protein subunit can be tested using a variety of methods known in the art. For example, recombinant proteins can be produced and their GTPase activity tested. Cell-based assays can be used.

The invention provides a method of detecting ion channel activity or lack thereof comprising steps of: (i) providing a cell comprising an ion channel having a nanosensor component attached thereto, wherein the nanosensor component comprises a nanoparticle; (ii) maintaining the cell under conditions in which ion channel activity may occur; and (iii) detecting a signal that is indicative of ion channel activity or lack thereof. The nanosensor component, either alone or in combination with one or more additional components or moieties, forms an ion-sensitive nanoscale sensor.

The nanoscale sensors of the invention can be employed to sense the activity of a wide variety of ion channels. Ion channels, for example, can be constitutive (i.e., their probability of being in the open state is high even in the absence of a stimulus), or their opening can be responsive to a stimulus such as a ligand or a change in voltage across the membrane. Many ion channels of interest herein permit the passage of one or more physiologically significant ions such as calcium ions (Ca⁺⁺), magnesium ions (Mg⁺⁺), sodium ions (Na⁺), potassium ions (K⁺), chloride ions (Cl⁻), or protons (H⁺). References to an element such as calcium, magnesium, sodium, potassium, chloride, or hydrogen herein, should be understood to refer to the physiologically relevant ionic form of that element unless otherwise indicated.

Ion channels of interest include, but are not limited to, acid-sensing (proton gated) ion channels, voltage-gated calcium channels, chloride channels, cyclic nucleotide-gated channels, epithelial sodium channels, hyperpolarising cyclic nucleotide channels, inositol 1,4,5 trisphosphate receptors, potassium channels, ryanodine receptor, voltage-gated sodium channels, and transient receptor potential (TRP) channels. Transmitter-gated ion channels include the nicotinic acetylcholine receptor, GABA_{A} receptor, glycine receptor, 5-hydroxytrypamine₃ receptor, and P2X receptors.

Calcium channels and methods of detecting their activity are exemplified herein, but the invention is in no way limited to calcium channels. Voltage-gated calcium channels alter their activity in response to a change in voltage across the plasma membrane. These channels typically form hetero-oligomeric complexes that include a pore-forming α1 subunit that includes four homologous repeats (I-IV), each repeat having six transmembrane (TM) domains. At least 10 α1 subunits have been cloned, and voltage-gated calcium channels have been divided into 3 families based on their α subunit. Under an alternative nomenclature, voltage-gated calcium channels can be classified as L-type, P/Q-type, N-type, R-type, and T-type on the basis of their physiological properties and/or tissue distribution. In addition to the α1 subunit, certain voltage-gated calcium channels also contain β and/or α2-γ subunit(s). A number of different β and α2-γ subunit genes have been cloned (92). Many of the α1 subunits give rise to alternatively spliced products, as do the other subunits.

TRP channels are believed to play a role in a wide variety of physiological processes, responding to temperature, touch, pain, osmolarity, pheromones, and other stimuli from both within and without the cell (84). The TRP superfamily of cation channels can be divided into seven families: TRPC, TRPM, TRPN, TRPV, TRPA, TRPP, and TRPML based on amino acid sequence homologies. The TRPC family includes TRPCI-TRPC7; the TRPM family includes TRPM1-TRPM8; the TRPV family includes TRPV1-TRPV6. Certain TRP channel subunits contain six putative TM domains and are believed to assemble as homotetramers or heterotetramers to form cation-selective channels. Certain of these channels are calcium-selective.

Further information regarding these and other ion channels, and their nomenclature, is found in Alexander, S P H, et al. (eds.), “Guide to Receptors and Channels”, Br. J. Pharmacol., Vol. 141, Supp. 1, 2004. The molecular, biophysical and pharmacological properties of identified mammalian sodium, calcium and potassium channels, as well as cyclic nucleotide-modulated ion channels and transient receptor potential (TRP) channels are also described in the International Union of Pharmacology (IUPHAR) publication entitled “The IUPHAR Compendium of Voltage-gated Ion Channels 2002”. Information on ion channels and receptors is also found in the official database of the IUPHAR Committee on Receptor Nomenclature and Drug Classification, available on the IUPHAR web site: www.iuphar-db.org/iuphar-ic/index.html (ion channels) and www.iuphar-db.org/iuphar-rd/index.html (receptors) (visited Oct. 27, 2005).

Certain ion channels are highly selective, i.e., they permit the passage of one ion to a much greater extent than other ions, while other ion channels are much less selective, and the invention includes embodiments in which a given nanosensor specifically detects a single ion type and embodiments in which a given nanosensor detects any of a plurality of different ion types.

Activity of a channel refers to the ability of a channel to allow passage of ions across a cell membrane such as the plasma membrane. Ion channel activity increases or decreases in response to a variety of stimuli which can be naturally occurring or artificially induced. Ion channel activity is generally detected or measured with respect to one or more ion types to which the channel is permeable in its open state. Increased ion channel activity will generally result in increased passage of ions through the channel and into the cell cytosol, which causes an increase in local ion concentration in the cytosol near the mouth of the channel (i.e., the cytoplasmic side of the ion channel pore). In certain embodiments of the invention one or more ion channel opening or closing events is detected. An ion channel opening event is typically accompanied by a rapid increase in local ion concentration in the cytosol near the opening of the channel. An ion channel closing event is typically accompanied by a decrease in local ion concentration in the cytosol near the channel mouth, as the ion diffuses away.

Ion channel activity may be expressed in a number of ways. For example, activity may be expressed in terms of (i) number of channel opening events per unit time; (ii) average duration of channel open state; (iii) total channel open time per unit time; (iv) number of ions passing through channel per unit time; (v) number of ions passing through channel per unit time during open state; (vi) number of cells in which one or more channel opening events occurs per unit time; (vii) channel opening and/or closing rate; and/or (viii) current through a channel. The methods of the invention may provide information about any of these or related parameters or others. The information may be an average obtained by monitoring a population of cells. Ion channel activity may be monitored over any suitable period of time, e.g., seconds, minutes, hours, or days. Typically the time period over which ion channel activity is assessed is at least several times the expected time interval between channel opening events. The information provided by the sensors described herein can be qualitative or quantitative.

GPCRs are transmembrane proteins having seven transmembrane domains. They represent the largest family of cell surface receptors involved in signal transduction across cellular membranes. These receptors physically associate with trimeric proteins known as G proteins, which mediate a diverse array of cellular activities and responses to internal and external stimuli. G proteins are complexes containing three different subunits (α, β, and γ), of which the a subunit is capable of binding to and hydrolyzing guanosine triphosphate (GTP). A variety of different G proteins are known and have been named in part based on whether they stimulate or inhibit downstream effector molecules. G proteins of interest include, e.g., Gₒ, Gᵢ, Gₛ, and G_{q}. GPCRs, G proteins, and their function, interactions, and regulation have been the subject of considerable study (reviewed in 90 and 91).

Coupling with G proteins is widely considered to be the defining characteristic of GPCRs, and it is by means of such coupling that information from activated (ligand-bound) cell surface GPCRs is relayed to intracellular signaling molecules, which mediate or activate downstream events. Ligand binding to GPCRs promotes or stabilizes conformational states of the receptor that favor engagement with the Gαβγ complex. As a consequence, guanosine diphosphate (GDP) is exchanged for GTP on the Gα subunit, leading to G protein diassembly. The free Gα and Gβγ subunits then interact with downstream effectors, either activating or inhibiting them depending on the particular G protein and effector under consideration. Among these effectors are a variety of ion channels and enzymes.

For purposes of the present invention it is important to note that GPCR activity, upon ligand binding, initially results first in engagement of the GPCR with the G protein, thereby altering the proximity between the two (e.g., decreasing the distance between the G protein and the Gα, Gβ, and/or Gγ subunits) and second in dissociation of the G protein a and βγ subunits both from each other and from the GPCR (increasing the distance between the GPCR and the Gα and Gβγ subunits and the distance between the Gα and Gβγ subunits). The methods of the invention detect one or more of these alterations in proximity, thereby detecting GPCR activity and thus also detecting the activity of the G protein with which the GPCR interacts and (indirectly) providing information about the activity of downstream targets of the G protein.

The nanoscale sensors of the invention can be employed to sense the activity of a wide variety of GPCRs. It is estimated that the human genome encodes between 340 and 400 GPCRs of possible pharmacological significance. Exemplary GPCRs and GPCR caterogies of interest include, but are not limited to, muscarinic acetylcholine receptor, adenosine receptors, adrenoceptors, calcium-sensing receptors, dopamine receptors, histamine receptors, metabotropic glutamate receptors, 5-hydroxytryptamine receptors, opioid and opioid-like receptors, P2Y receptors, tachykinin receptors, etc. The foregoing list is provided merely to offer a selection of the wide range of GPCRs of interest herein. Many of these receptor types represent families having multiple members. It should be noted that the endogenous ligand(s) of many GPCRs of interest have not yet been identified. Further information regarding these and other receptors, and their nomenclature, is found in Alexander, SPH, et al. (eds.), “Guide to Receptors and Channels”, Br. J. Pharmacol., Vol. 141, Supp. 1, 2004.

The invention provides a method of method of detecting GPCR activity or lack thereof comprising steps of: (i) providing a cell comprising a GPCR having a nanosensor component attached thereto, wherein the nanosensor component comprises a nanoparticle; (ii) maintaining the cell under conditions in which GPCR activity may occur; and (iii) detecting a signal that is indicative of GPCR activity or lack thereof. The nanosensor component, either alone or in combination with one or more additional components or moieties, forms a nanoscale sensor capable of detecting proximity of two or more polypeptides.

Among the important aspects of the invention are (i) the selection of particular pairs of recognition domains and target domains; (ii) engineered polypeptides comprising the recognition domains; (iii) cells that synthesize an engineered polypeptide in which ion channel or GPCR activity or physical interaction of polypeptides is to be detected; (iv) the design and implementation of nanosensor components, nanoscale sensors, and detection methods; (v) production of nanosensor components comprising the target domains and adapted for sensing ions and/or physical interactions; (vi) achieving uptake of the nanosensor components by cells; (vii) methods of using the nanosensor components to identify compounds that modulate the activity of ion channels or receptors such as GPCRs. These and other aspects of the invention are described in the following sections.

II. Recognition Domains and Target Domains

As noted above, the selection of particular pairs of recognition domains and target domains such that a nanosensor component comprising a particular recognition domain will bind to a predetermined polypeptide comprising a target domain is an important aspect of the invention. A “recognition domain” is a molecule or portion thereof that is capable of specifically interacting with and binding to a second molecule or portion thereof. A “target domain” is a portion of the second molecule with which the recognition domain specifically binds. Recognition and target domains that specifically bind to one another are referred to as “specific binding pairs” and are said to “correspond” to one another. The recognition and target domains of a suitable specific binding pair should specifically and stably bind to one another under physiological conditions. In certain embodiments of the invention the recognition domain does not substantially bind to any cellular polypeptide other than the polypeptide of interest. For example, the K_{d} of the recognition domain and any other cellular polypeptide may be at least 10-fold greater, at least 100-fold greater, or at least 1000-fold greater than the K_{d} of the recognition domain and the target domain.

Preferably members of a specific binding pair bind to one another with high affinity. For example, the dissociation constant (K_{d}) of a complex in which a recognition domain and target domain are bound may be 50 μm or less, 10 μm or less, preferably 1 μm or less. In certain embodiments of the invention the recognition domain and the target domain are different, such that two molecules, each of which contains the recognition domain, do not bind to one another, and two molecules each of which contains the target domain do not bind to one another (i.e., the recognition domain does not form homodimers and the target domain does not form homodimers).

A variety of different molecules or portions thereof may serve as recognition and/or target domains. A recognition domain can be or comprise an organic or non-organic molecule or molecular complex. A recognition or target domain can be identical to a molecule or molecular complex found in nature or can have a structure invented by man. For example, recognition and target domains can be polypeptides, nucleic acids, carbohydrates, lipids, or small molecules.

The recognition domain can be produced in a wide variety of ways depending on its particular composition. For example, it can be purified from natural sources, synthesized using enzymatic and/or chemical synthesis procedures, etc. In certain embodiments of the invention the target domain is tsynthesized by a cell in which protein activity or interaction is to be detected, using the biosynthetic processes that can take place in the cell, which include protein synthesis, post-translational modification, etc., and by the substrates that are provided to the cell. Such substrates can include naturally occurring and/or non-naturally occurring compounds (e.g., amino acids analogs that are not found in nature, etc.) The cell may also be modified to include synthetic machinery or substrates that are not naturally found in the cell. For example, the cell may be genetically engineered to express biosynthetic enzymes that it does not naturally express. In other embodiments, ion channel subunits are synthesized (e.g., by enzymatic or chemical methods) and are transferred either to the plasma membrane of living cells or to artificial lipid bilayer membranes. A wide range of target domains including polypeptide and non-polypeptide target domains can be used in such synthesized ion channel subunits. In certain embodiments the channel is provided with a pre-attached nanosensor and is directed to the plasma membrane of a cell either in vitro or in vivo, e.g., to assess its activity.

In certain embodiments of the invention the recognition and/or target domain is artificial, i.e., it is not found in nature, or may comprise an artificial portion. In certain embodiments of the invention the recognition and/or target domain is not found in a cell in which the predetermined polypeptide is expressed, e.g., it is not synthesized by the cell or naturally present within the cell (e.g., is not encoded in the genetic information of such cells, is not synthesized by the cell, and/or is not present within the cell as a nutrient, metabolite, waste product, etc.). For example, for purposes of detecting activity or physical association of mammalian polypeptides, a molecule or portion thereof found in viruses, in bacteria, or in non-mammalian eukaryotes (e.g., fungi such as yeast, insects, plants, etc.), may be used as a recognition or target domain. In certain embodiments of the invention the target domain is not naturally found within a cell in which the polypeptide containing the target domain is expressed.

In certain embodiments of the invention the recognition domain, target domain, or both, are polypeptides. A polypeptide may be referred to herein as a polypeptide portion or segment to indicate that the polypeptide is or can be part of a longer polypeptide. In certain embodiments of the invention the target domain is a polypeptide segment that does not naturally occur in the polypeptide of interest. In certain embodiments of the invention the recognition domain, target domain, or both, are polypeptides or polypeptide segments that do not naturally occur in the polypeptide of interest. A recognition or target domain can be a portion of a larger molecule. For example, a recognition domain can be a portion of a longer polypeptide that is attached to a nanoparticle. A recognition domain or a target domain can be a carbohydrate or lipid moiety attached to an amino acid side chain of a polypeptide or polypeptide segment.

In certain embodiments of the invention the target domain, recognition domain, or both, are polypeptides that are heterologous to the cell in which activity or interaction of the polypeptide of interest is to be detected, i.e., the domain(s) do not occur in any polypeptide naturally produced by the cell. Preferably heterologous recognition and target domains do not specifically bind to any native cellular polypeptide, i.e., any polypeptide that is naturally produced by the cell in which activity or interaction is to be detected. The sequence of a heterologous target domain may be one that is not found in nature (although a portion of the sequence may be found in nature).

In certain embodiments of the present invention, the target domains are heterologous polypeptide domains that exist within recombinant polypeptides (e.g., ion channel subunits) produced by cells that are specifically engineered so that they synthesize the recombinant polypeptides. The cells are engineered to synthesize the recombinant polypeptides using methods well known in the art, e.g., by introducing a nucleic acid encoding the recombinant polypeptide into the cells, which can be done according to standard techniques. Stable cell lines expressing the recombinant polypeptide can be generated, or cells that transiently express the recombinant polypeptide can be employed.

In certain embodiments of the invention a recognition or target domain comprises at least 2, at least 3, at least 5, at least 10, or at least 20 amino acids. In certain embodiments of the invention a recognition or target domain comprises not more than 30 amino acids, not more than 50 amino acids, not more than 100 amino acids, or not more than 200 amino acids. In certain embodiments of the invention the length of a recognition or target domain is any integer between 2 and 200. In various embodiments of the invention a recognition domain may bind to any portion of a target domain. In certain embodiments of the invention a recognition domain may bind to a carbohydrate that modifies an amino acid within the target domain.

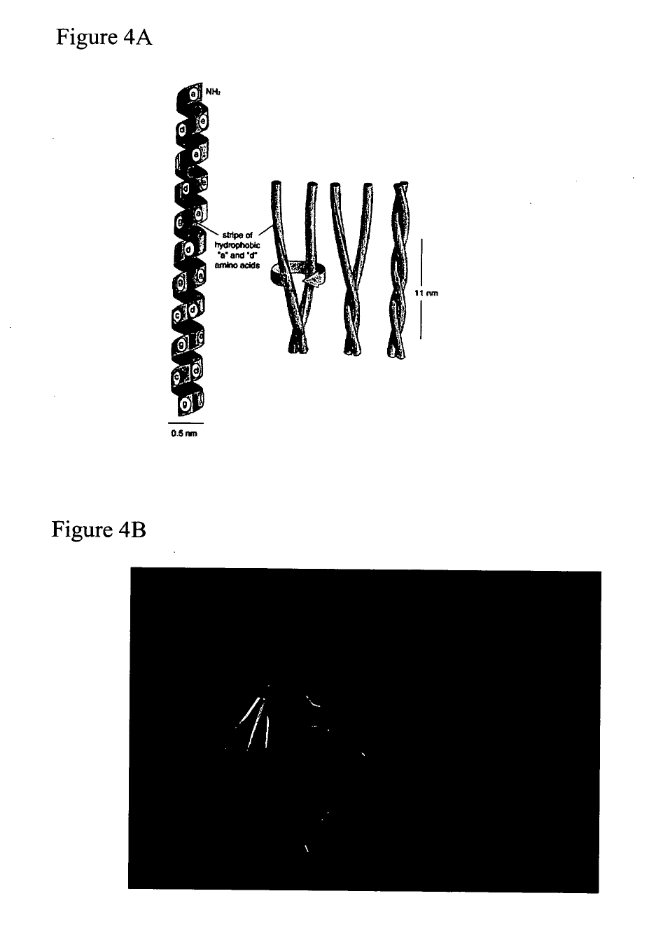

In certain embodiments of the invention the recognition domain and the target domain each comprise an α-helical peptide capable of forming a coiled-coil structure. The coiled-coil is one of the most important protein-protein recognition motifs in nature, occurring in proteins as diverse as motor proteins, DNA binding proteins, extracellular proteins, and viral fusion proteins (FIG. 4A). Examples of proteins that form coiled-coils include, e.g., GCN4, tropomyosin, cortexillin I, Fos, and Jun. A coiled-coil typically consists of two α-helices that wrap around each other to form a super-helix in which the side chains of the helices pack together in a so-called “knobs-into-holes” manner. The most common coiled-coil is left-handed, with each helix being characterized by a “heptad repeat” in its amino acid sequence: (abcdefg)ₙ, where the a and d amino acids are hydrophobic (e.g., leucine, valine, isoleucine), while e and g are charged or polar (e.g., glutamate or lysine), and n represents the number of repeats in the sequence. Each of the coiled-coil helices can contain between 2 and about 200 such repeats. Right handed coiled-coils with an eleven-residue repeat are also known.

A peptide or polypeptide that is capable of interacting to form a coiled-coil with an appropriate second peptide or polypeptide is referred to herein as a “coiled-coil” peptide or polypeptide. Coiled-coil peptides or polypeptides that are capable of interacting with each other to form a coiled-coil are referred to as “complementary”. Coiled-coil peptides or polypeptides may contain only amino acids that are found naturally in proteins or may contain amino acids or amino acid analogs that do not naturally occur in proteins.

The coiled-coil motif has been the object of considerable study (see, e.g., references 48-50 for review). The three-dimensional structure of a number of coiled-coils has been solved; moreover, the three-dimensional structure of this motif can be reliably predicted from its amino acid sequence. Furthermore, computer programs are available to identify coiled-coil domains, e.g., Socket (53) and Twister (54). The ability of any two polypeptides to form a coiled-coil can be assessed using methods known in the art. For example, the secondary structure, oligomerization state, and thermodynamic stability of polypeptides can be assessed by circular dichroism (CD), sedimentation equilibrium, and or chemical unfolding experiments.

Coiled-coil domains have been designed de novo, e.g., in order to obtain desired properties, such as high binding affinity or specificity. For example, a variety of artificial coiled-coil peptides whose complete sequence is not found in nature have been designed and their binding properties assessed (17, 51, 52). Certain coiled-coil peptides form homodimers while others form heterodimers, depending upon their particular interhelical electrostatic interactions (49).

A wide variety of coiled-coil peptides including both naturally occurring coiled-coil peptides (which are typically found within larger polypeptides) and artificial coiled-coil peptides can be employed. In certain embodiments of the invention the recognition domain and/or the target domain comprises a coiled-coil peptide that does not form homodimers.

In one embodiment the recognition domain comprises at least two repeats of the heptad unit (KVSALKE) (SEQ ID NO: 1), and the target domain comprises at least two repeats of the complementary heptad unit (EVSALEK) (SEQ ID NO: 2). In another embodiment the target domain comprises at least two repeats of the heptad unit (KVSALKE) (SEQ ID NO: 1), and the recognition domain comprises at least two repeats of the complementary heptad unit (EVSALEK) (SEQ ID NO: 2). Each domain may comprise 2, 3, 4, 5, 6 or more heptad repeats. In other embodiments the recognition and target domains each comprise at least 3 heptad repeats, e.g., between 3 and 6 heptad repeats. For example, in one embodiment the recognition domain comprises three repeats, e.g., (KVSALKE)₃ (SEQ ID NO: 3) and the target domain comprises four heptad repeats, e.g., (EVSALEK)₄ (SEQ ID NO: 4). In another embodiment the target domain comprises three repeats, e.g., (KVSALKE)₃ (SEQ ID NO: 3) and the recognition domain comprises four heptad repeats, e.g., (EVSALEK)₄ (SEQ ID NO: 4).

FIG. 4A schematically depicts a nanosensor component comprising a nanoparticle functionalized with a coiled-coil peptide and an ion channel subunit comprising the complementary coiled-coil peptide and their interaction within a cell. It will be appreciated that the N- to C-orientation of the peptide with respect to the nanoparticle or ion channel subunit is significant and should be appropriate to allow superhelix formation. In vitro experiments suggest that a coiled-coil formed by these peptides is predicted to have a K_{d} of ˜3 μM (17).

In another embodiment recognition and target domains based on the IAAL-E3/K3 coiled-coil are employed. The IAAL-E3/K3 coiled-coil is a highly stable heterodimer (dissociation constant 70 nM) (78). The sequences of the complementary peptides are: EIAALEK-EIAALEK-EIAALEK (SEQ ID NO: 5) and KIAALKE-KIAALKE-KIAALKE (SEQ ID NO: 6) These two peptides assemble into a parallel coiled-coil.

In another embodiment, recognition and target domains based on the the so-called “Velcro” heterodimer (79) are employed. The sequences of these coiled-coil peptides are AQLEKE-LKALEQE-NAQLEWE-LQALEKE-LAQ (SEQ ID NO: 7) and AQLKKK-LQALKKK-NAQLKWK-LQALKKK-LAQ (SEQ ID NO: 8)

In another embodiment coiled-coil peptides based on the Jun-Fos heterodimer are used (80). The sequences are IARLEEK-VKTLKAQ-NSELAST-ANMLREQ-VAQL (SEQ ID NO: 9) and TDTLQAE-TDQLEDE-KSALQTE-IANLLKE-KEKL (SEQ ID NO: 10)

Yet another embodiment of the invention employs recognition and target domains based on the WinZip-A2B1 heterodimer (81), whose sequences are: VAQLRER-VKTLRAQ-NYELESE-VQRLREQ-VAQL (SEQ ID NO: 11) and VDELQAE-VDQLQDE-NYALKTK-VAQLRKK-VEKL (SEQ ID NO: 12).

The coiled-coil recognition and target domains may be attached to the nanoparticle and inserted into the polypeptide of interest in a variety of different combinations, which are depicted schematically below. Here n represents the N-terminus, c represents the C-terminus of either a coiled-coil peptide or a channel, P represents a nanoparticle, and c1 and c2 are the two a-helices forming the coiled-coil (i.e., the recognition and target domains).

P-c1-c

n-c2-channel-c

P-c2-c

n-c1-channel-c

n-channel-c1-c
n-c2-P

n-channel-c2-c
n-c1-P

The recognition domain and/or target domain may contain one or more amino acids that do not participate in formation of a coiled-coil. For example, it may be desirable to include a spacer (e.g., CGG) at one end of the recognition domain to facilitate its attachment to a nanoparticle. The spacer can be, e.g., between 1 and 10 amino acids in length, e.g., 1, 2, 3, 4, or 5 amino acids in length. Attachment can be achieved using a variety of approaches that are known in the art and discussed elsewhere herein. In addition, one or more modifications may be made to the recognition domain peptide, e.g., acetylation, amidation, etc., e.g., to increase stability by neutralizing the charge at the termini.

In certain embodiments of the invention the recognition domain and the target domain are an enzyme/enzyme inhibitor pair. Either or both the enzyme and enzyme inhibitor may be polypeptides or portions thereof. The enzyme may be, for example, a protease or nuclease, e.g., an endonuclease or exonuclease. In certain embodiments of the invention the enzyme and enzyme inhibitor do not naturally occur in the cell in which ion channel activity or protein interaction is to be assessed.

Many instances are known in which an endogenous enzyme is maintained in an inactive state by complexation with an endogenous inhibitor. For example, serpins are a family of proteins that inhibit a wide variety of different serine proteases (55). The interacting domains of a number of different serpins and the proteases that they inhibit have been identified. Thus a serpin and a protease inhibited by the serpin (or portions thereof) can be used as complementary recognition and target domains. Every mammalian cell studied contains a cytosolic ribonuclease (RNase) inhibitor (R1) that binds to RNase A and other members of the RNase A superfamily with high affinity (see 56 for review). The binding of R1 to ribonucleases results in the complete loss of ribonucleolytic activity. In certain embodiments of the invention an RNase and an RNase inhibitor, e.g., RNase A and R1, are used as complementary recognition and target dmoains.

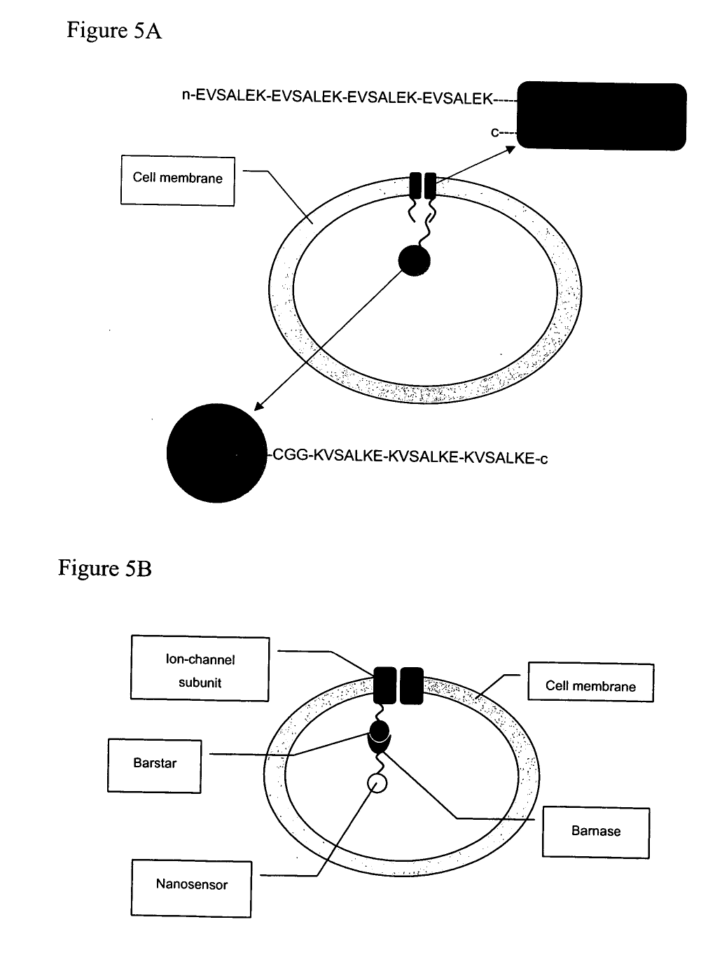

In one embodiment, barnase and barstar are used as the recognition domain and target domain. Barnase is a bacterial ribonuclease protein and barstar is its inhibitor (43). Both are approximately 100 amino acids long, highly soluble and stable as monomers (FIG. 4B). Upon encounter, they rapidly form a complex with quasi-covalent affinity (dissociation constant K_{d} ˜10⁻¹⁴M). In one embodiment depicted schematically in FIG. 5B, a recombinant ion channel subunit comprising barstar (e.g., fused at the N-terminus) is expressed in a cell. Barnase is attached to the nanosensor component.

A variety of changes (e.g., amino acid substitutions, additions, deletions) may be made to certain of the recognition and/or target domains described herein (and others), provided that such changes do not prevent or substantially diminish the ability of the domains to specifically bind to one another. In certain cases, such changes may increase binding affinity. One or more alterations (e.g., substitutions, deletions, or insertions) can be made to the sequence of a naturally occurring or artificial recognition or target domain or portion thereof using standard recombinant DNA techniques. One of ordinary skill in the art will be able to engineer a number of variants and test their binding affinity, e.g., using standard in vitro or in vivo binding assays. Such altered versions of recognition and/or target domains, wherein the binding affinity of a binding pair comprising either (i) an altered recognition domain and an altered target domain; or (ii) an altered recognition domain and an original target domain; or (iii) an original recognition domain and an altered target domain, is substantially similar to that of the original binding pair, are considered equivalents of the original domains. For example, the binding affinity may be within a factor of 10 of the original binding affinity, or within a factor of 100 of the original binding affinity. In certain embodiments of the invention such alterations result in a variant whose sequence is at least approximately 90% identical to the original sequence (which may be either a full length sequence or a portion of the sequence). In certain embodiments not more than 10 amino acid changes are made (e.g., 1, 2, 3, 4, or 5 amino acid changes are made) i.e., not more than 10 amino acids are substituted, added, or deleted. It may be preferable to make conservative amino acid substitutions. For purposes of the present invention conservative replacements may be defined in accordance with Stryer, L,. Biochemistry, 3rd ed., 1988, according to which amino acids in the following groups possess similar features with respect to various side chain properties (1) Aliphatic side chains: G, A, V, L, I; (2) Aromatic side chains: F, Y, W; (3) Sulfur-containing side chains: C, M; (4) Aliphatic hydroxyl side chains: S, T; (5) Basic side chains: K, R, H; (6) Acidic amino acids: D, E, N, Q; (7) Cyclic aliphatic side chain: P, which may be considered to fall within group (1). Alternately it may be desirable to replace nonpolar (hydrophobic) amino acids (G, A, V, L, I, F, W, M, P) with other such amino acids, neutral amino acids with polar side chains (S, T, C, Y, N, Q) with other such amino acids, acidic amino acids (D, E) with other such amino acids, and/or basic amino acids (K, R, H) with other such amino acids.

For example, in certain embodiments of the invention a variant of a naturally occurring enzyme or enzyme inhibitor or portion thereof is employed. In certain embodiments of the invention the alteration substantially reduces the enzymatic activity of the enzyme (e.g., by at least 75%, preferably at least 90%, 95%, or 99%) or even to an undetectable level. Typically such alterations result in a variant whose sequence is at least approximately 90% identical to the original sequence (which may be either a full length sequence or a portion of the sequence). Preferably the alteration(s) do not substantially reduce the affinity of the enzyme and the inhibitor.

In some embodiments either the recognition domain or the target domain comprises an active enzyme, which may be toxic to a cell. However, binding of the enzyme to a complementary recognition or target domain comprising an inhibitor of the enzyme renders the enzyme inactive, thereby providing a selection method: most or all cells alive for observation would have a polypeptide of interest successfully bound to a nanosensor component.

In another embodiment the recognition domain and the target domain are an enzyme/substrate analog pair, where a “substrate analog” refers to a molecule that structurally resembles a substrate of an enzyme such that the substrate binds to the enzyme (e.g., at its active site) but does not actually serve as a substrate, instead remaining bound to the enzyme. The substrate analog may be a naturally occurring substrate analog or pseudosubstrate, which may be a portion of a polypeptide that contains an enzymatically active portion.

In another embodiment the recognition domain and target domain are naturally found as subunits of a protein containing multiple polypeptide subunits, etc. In another embodiment the recognition domain and target domain are polypeptides or portions thereof that specifically bind to one another and are found naturally in a cell other than the one in which activity or interaction is to be assessed. One of ordinary skill in the art will be able to generate a variety of different designs employing different specific binding pairs using the teachings herein.

In certain embodiments of the invention the recognition domain is not an antibody or epitope-binding portion thereof. However, in other embodiments of the invention the recognition domain may be an antibody. For example, if the nanosensors are to be attached to the outside-facing portion of an ion channel or receptor subunit, antibodies that bind to domains that naturally occur in the polypeptide may be used. Antibodies that bind to many of the polypeptides of interest herein are well known in the art. In the case where the nanosensor component is to be delivered inside cells, it may be preferable to avoid use of recognition domains that contain disulfide bonds that are needed for maintenance of secondary structure and/or for the ability of the recognition domain to specifically bind to its corresponding target domain since such bonds may be reduced under physiological conditions, e.g., within cells. In certain embodiments of the invention the recognition domain and the target domain are not biotin, avidin, or streptavidin, although this possibility is not excluded. In preferred embodiments of the invention the recognition domain is not a protein or molecule that is typically used as a coating to render nanoparticles biocompatible, such as bovine serum albumin (BSA), bovine gamma globulin, polyethylene glycol (PEG), etc.

A variety of different natural or engineered polypeptide domains that specifically bind to other polypeptides or to small molecule ligands are known in the art and can be used as recognition and/or target domains in the present invention. See 16 for review, and references therein. For example polypeptides based on the ankyrin repeat motif may be used (58). Affibodies are engineered binding proteins based on the three-helix scaffold of the Z domain derived from staphylococcal protein A. (59). Anticalins are another class of engineered ligand-binding proteins with tailored specificities and are derived from the lipocalin protein scaffold (60). Anticalins that bind to a variety of small molecules such as fluorescein and digoxigenin have been designed and are of use in the present invention.

In certain embodiments of the invention the recognition domain is a polypeptide segment that specifically binds to a native polypeptide of interest such as an ion channel subunit, G protein subunit, or GPCR. Recognition domains that specifically bind to a polypeptide or portion thereof can be identified using a variety of selection methods known in the art. For example, phage display, bacterial display, ribosome display and similar display technologies have been used to successfully identify peptides or polypeptides that bind to a wide variety of different polypeptides (see, e.g., 57 and U.S. Pat. Nos. 5,885,793 and 5,969,108). Such peptides or polypeptides can be selected to bind to a particular portion of the polypeptide of interest. Rational design based on structural information has also been successfully used to design polypeptides that bind to a variety of different target proteins. It may be desirable to select as a target domain a portion that is unique to the polypeptide of interest and is not highly homologous to other cellular polypeptides produced by cells in which the polypeptide activity or interaction is to be detected. Selection of such a target domain can readily be performed by searching a publicly available database such as GenBank with the sequence of the polypeptide of interest to identify regions that do not have close homologs or by selecting a candidate target domain and searching the database specifically with that target domain.

In certain embodiments of the invention the recognition domain is a nucleic acid. A variety of different nucleic acids may serve as recognition domains. In certain embodiments of the invention the recognition domain is an aptamer. An aptamer is an oligonucleotide that binds to a particular polypeptide. Aptamers are typically derived from an in vitro evolution and selection process called SELEX, and methods for obtaining aptamers specific for a polypeptide of interest are known in the art. See, e.g., Brody E N, Gold L. J Biotechnol. 2000 March; 74(1):5-13. Methods for efficient selection of aptamers that bind to any polypeptide of interest are described in U.S. Pub. No. 20050142582. See also (47) and references therein.

The oligonucleotide can comprise only nucleosides such as those found in naturally occurring nucleic acids (RNA or DNA), or can contain one or more modified nucleosides, e.g., nucleosides that confer increased stability or increased binding affinity on the oligonucleotide. The nucleosides can comprise a modified base or sugar moiety. The oligonucleotide backbone can contain phosphodiester linkages, or the oligonucleotide can contain a modified phosphodiester backbone or non-naturally occurring internucleoside linkages e.g., phosphorothioate or phosphoramidite linkages. See Kornberg and Baker, DNA Replication, 2nd Ed. (Freeman, San Francisco, 1992), Scheit, Nucleotide Analogs (John Wiley, New York, 1980), U.S. Patent Pub. No. 20040092470 and references therein for further discussion of various nucleotides, nucleosides, and backbone structures that can be used in an oligonucleotide and methods for producing them.

In one embodiment, the aptamer binds to a heterologous recognition domain of an engineered polypeptide such as an ion channel subunit, G protein, or receptor polypeptide. In another embodiment the aptamer binds to a naturally occurring portion of a polypeptide such as an ion channel subunit, G protein, or receptor polypeptide.

Polypeptide domains (e.g., homeodomains) that bind to specific nucleic acid sequences are also known. Such nucleic acids may be used as recognition domains, wherein the corresponding target domain comprises a homeodomain. Homeodomains occur naturally in a variety of proteins (e.g., transcription factors) and typically comprise about 60 amino acids and contains a DNA-binding helix-turn-helix motif. Artificial homeodomains have been designed and are of use as target domains in certain embodiments of the invention.

In certain embodiments of the invention the recognition domain is a small molecule. Small molecules that bind to a number of different polypeptides are known and could be used as recognition domains provided that the polypeptide or portion thereof to which the small molecule binds is used as a target domain in a polypeptide of interest such as an ion channel subunit. For example, hormones such as glucorticoids and other steroids (e.g., estrogen) bind to specific polypeptide receptors. Other examples include transferrin, which binds to the transferrin receptor. Another example is the binding of tetracycline and related antibiotics to the TET repressor protein. Such small molecules may be used as the recognition domain provided that the appropriate target domain is present in the polypeptide of interest. If the small molecule is also used to regulate expression of the polypeptide of interest it may be desirable to avoid using it also as a recognition domain although in some embodiments of the invention a small molecule is used for both purposes. Screening of small molecule libraries, e.g., combinatorial or natural product libraries can be performed using in vitro assays such as binding assays, and/or computer-based screening, e.g., to identify small organic compounds that bind to a polypeptide of interest. See, e.g., 38 for examples of the use of such methods to identify small organic compounds that bind to concave surfaces (pockets) of a variety of polypeptides.

III. Recombinant Polypeptides and Cells that Express them

The invention provides a recombinant polypeptide comprising (i) an ion channel subunit, GPCR, or G protein subunit and (ii) a heterologous target domain. The ion channel subunit can be a subunit of any of the ion channels known in the art or discovered in the future including, but not limited to, those mentioned above. For example, in certain embodiments of the invention the ion channel subunit is a calcium channel subunit, e.g., a voltage-gated calcium channel subunit, a TRP channel subunit, or a P2X2 channel subunit. The GPCR can be any of the GPCRs known in the art or discovered in the future including, but not limited to, those mentioned above. The G protein subunit can be any G protein subunit known in the art or discovered in the future. For example, the G protein subunit can be an α, β, or γ subunit of a G protein selected from the group consisting of Gₒ, Gᵢ, Gₛ, and G_{q}.

The polypeptide of interest can be from any species. In certain embodiments of the invention the polypeptide is from a mammalian species, e.g., human, non-human primate, rat, mouse, rabbit, etc. The target domain in any of the recombinant polypeptides can be any of the target domains discussed above.

The invention provides an ion channel comprising one or more of any of the recombinant ion channel subunits described herein, e.g., an ion channel subunit comprising one or more of the target domains described herein. The ion channel may span an artificial lipid bilayer or in a cell membrane. The invention further provides a a GPCR comprising one or more of the target domains described herein. G protein comprising one or more recombinant G protein subunits described herein, e.g., a G protein subunit comprising one or more of the target domains described herein.

The invention provides a nucleic acid that encodes any one or more of the recombinant polypeptides. The nucleic acid can be a nucleic acid construct such as a vector. The nucleic acids and polypeptides of the invention can be provided in isolated and/or purified form. For example, they can be substantially free of other nucleic acids, polypeptides, cellular material, chemicals or chemical precursors used for synthesis, etc.

The nucleic acids and polypeptides have a number of uses in addition to the uses described herein. For example, the nucleic acids can be used to produce transgenic non-human animals that express the recombinant polypeptides. Such transgenic animals are also an aspect of this invention. The transgenic animals can be, for example, mice, rats, or other animals suitable for the study of ion channel activity, receptor activity, or protein interaction. The animals can be used to characterize the effects of various agents, e.g., therapeutic agents, potential therapeutic agents, toxins, etc., on the activity of the polypeptide of interest. Both the agent of interest and one or more nanoscale sensor components of the invention are administered to the animal. The characterization can be performed by removing samples from the animal, using whole body imaging (e.g., of a fluorescent or magnetic signal), etc., to gather a signal that provides information indicative of ion channel or receptor activity, protein interaction, etc. The polypeptides can be used as antigens for the production of antibodies or can be used in in vitro binding assays to identify polypeptides or compounds (e.g., potential therapeutic agents or potential targets for therapeutic agents) that bind to them.

The invention further provides a cell that expresses one or more of the recombinant polypeptides. The cell may, for example, express 1, 2, 3, or more different recombinant ion channel subunits. The recombinant subunits may be subunits of any of ion channel species. The recombinant ion channel subunits may be subunits of the same ion channel or of different ion channels. The cell may express one or more recombinant GPCRs and one or more recombinant G protein subunits. The cell may express one or more recombinant ion channel subunits, one or more recombinant GPCRs, and one or more recombinant G protein subunits. The cell can be provided in isolated form, e.g., in culture or in a form suitable for culture as an individual cellular entity. Alternately, the cell can be part of a non-human transgenic animal.

The cell can be from any species. In certain embodiments of the invention the cell is from a mammalian species, e.g., human, non-human primate, rat, mouse, rabbit, etc.

Recombinant DNA techniques well known in the art are employed to produce a polypeptide of interest comprising a target domain. See, e.g., Current Protocols in Molecular Biology, Current Protocols in Immunology, Current Protocols in Protein Science, Sambrook, et al, supra. Briefly, in an exemplary method, nucleic acids encoding the polypeptide of interest and the target domain are obtained from any suitable source (e.g., by cloning from a cDNA library, by PCR amplification using appropriate primers and a template such as mammalian cDNA or a plasmid containing the coding sequence for the polypeptide, etc.). The nucleic acids can also be chemically synthesized based on the known sequence of the polypeptide and target domain. The nucleic acids encoding the polypeptide of interest and the target domain are inserted in frame with one another into a vector containing expression control signals suitable for directing expression in cells of the desired type, e.g., mammalian cells. They may be inserted as a single unit or as individual units in frame with one another. It will be appreciated that the details by which any particular nucleic acid construct is made can vary, and any suitable method can be employed.

The resulting vector typically includes a transcriptional unit comprising (i) genetic element(s) having a regulatory role in gene expression, e.g., expression control sequences such as, promoters, operators, or enhancers, operatively linked to (ii) a “coding” sequence which is transcribed to produce an RNA that can be translated into a polypeptide, and (iii) appropriate transcription initiation and termination sequences. In certain embodiments of the invention, expression of the recombinant polypeptide is regulatable, e.g., its expression is under control of a regulatable promoter. Suitable inducible or repressible promoters are known in the art. For example, certain promoters are inducible by heavy metals, hormones, small molecules, etc. In one embodiment, the TET system is used. As noted above, the target domain can be located anywhere in the polypeptide of interest. In some embodiments of the invention the position of the heterologous target domain is selected so that it will only be present in certain alternatively spliced variants of the polypeptide.

Cells that express the recombinant polypeptide are produced using methods well known in the art. The vector is typically introduced into cells of the desired type using art-recognized methods such as electroporation, lipid or calcium-mediated transfection, etc.), and cells are maintained under conditions in which the recombinant polypeptide containing a polypeptide of interest and a heterologous target domain is expressed. Cells can be transiently tranfected with the vector or stable cell lines can be generated using standard selection methods. A cell has been “stably transfected” with a nucleic acid construct when the nucleic acid construct is capable of being inherited by daughter cells. “Transient transfection” refers to cases where exogenous DNA does not integrate into the genome of a transfected cell, e.g., where episomal DNA is transcribed into mRNA and translated into protein.

In general, the recombinant polypeptides can be expressed in a wide variety of different cell types. Commonly used host cells include, but are not limited to, CHO, R1.1, B-W, L-M, COS-1, COS-7, BSC-1, BSC-40, BMT-10, BHK, HeLa, HEK-293, NIH/3T3, HT1080, HEK-293T, WI-38, and CV-1. For an extensive list of mammalian cell lines, those of ordinary skill in the art may refer to the American Type Culture Collection catalog (ATCC®, Manassas, Va.).

The cell may or may not naturally express the polypeptide of interest. In certain embodiments the cell does not express the natural form of the polypeptide of interest, e.g., the cell may have a natural or engineered mutation in the gene that encodes the polypeptide or may simply not express the polypeptide.

In certain embodiments of the invention, for purposes of detecting ion channel activity, the cell expresses all subunits required for formation of a functional ion channel. In other embodiments the cell does not express one or more ion channel subunits. Any subunit(s) not naturally expressed by the cell can be provided by transfection of an expression vector encoding the missing subunit(s). One use of the invention is to determine which particular subunits are needed for formation of a functional ion channel and/or to assess the properties of ion channels that assemble in the absence or presence of different subunits, e.g., in order to determine which subunits are needed for formation of a functional channel.

Cells, as described above, containing one or more nanosensor components described herein, are an aspect of the invention.

IV. Nanoparticles and their Production

The present invention features nanosensor components that comprise a nanoparticle. A variety of different nanoparticles are of use in different embodiments of the invention as described in more detail in Section V.

In certain embodiments of the invention a nanosensor component comprises a metal nanoparticle, optionally with a coating layer composed of one or more organic molecules, which may stabilize the particles (e.g., under physiological conditions), prevent spontaneous aggregation, improve solubility, etc. Metals of use in the nanoparticles include, but are not limited to, gold, silver, iron, cobalt, zinc, cadmium, nickel, gadolinium, chromium, copper, manganese, palladium, tin, and alloys thereof. Oxides of any of these metals can also be used.

Noble metals (e.g., gold, silver, copper, platinum, palladium) are preferred for sensors that utilize the phenomenon of plasmon resonance, discussed below. For these purposes gold and silver are particularly preferred. Alloys comprsing gold, silver, and optionally one or more other metals can also be used. In addition, core/shell particles (e.g., having a silver core with an outer shell of gold, or vice versa) can be used. Particles containing a metal core and a nonmetallic inorganic or organic outer shell, or vice versa, can also be used and are preferred in certain embodiments of the invention. In certain embodiments the nonmetallic core or shell comprises or consists of a dielectric material such as silica. Composite particles in which a plurality of metal particles are embedded or trapped in a nonmetal (e.g., a polymer or a silica shell) may also be used. Hollow metal particles (e.g., hollow nanoshells) having an interior space or cavity are used in some embodiments. In other embodiments a nanoshell comprising two or more concentric hollow spheres is used. Such a nanoparticle optionally comprises a core, e.g., made of a dielectric material.

In certain embodiments of the invention at least 1%, or typically at least 5% of the mass or volume of the particle or number of atoms in the particle is contributed by atoms of one or more metals. In certain embodiments of the invention the amount of metal in the particle, or in a core or coating layer comprising a metal, can range from approximately 5% to 100% by mass, volume, or number of atoms, or can assume any integer value or range between 5 and 100%. Such values and ranges will not be set forth explicitly herein.

In certain embodiments of the invention the particles are magnetic. The term “magnetic particle” refers to magnetically responsive particles that contain one or more metals or oxides or hydroxides thereof. Such particles typically react to magnetic force resulting from a magnetic field. The field can attract or repel the particle towards or away from the source of the magnetic field, respectively, optionally causing acceleration or movement in a desired direction in space. The term “magnetic” encompasses ferrimagnetic, ferromagnetic, paramagnetic, and superparamagnetic materials. Useful particles may be made entirely or in part of one or more materials selected from the group consisting of: iron, cobalt, nickel, niobium, magnetic iron oxides, hydroxides such as maghemite (γ-Fe₂O₃), magnetite (Fe₃O₄), feroxyhyte (FeO(OH)), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the afore-mentioned oxides or hydroxides, and mixtures of any of the foregoing. See, e.g., U.S. Pat. No. 5,916,539 for suitable synthesis methods for certain of these particles.

Additional materials that may be used in magnetic particles include yttrium, europium, and vanadium. Certain lanthanide ion-doped nanoparticles exhibit strong fluorescence and are of use in certain embodiments of the invention. A variety of different dopant molecules can be used (20). For example, fluorescent europium-doped yttrium vanadate (YVO₄) nanoparticles have been produced (21). These nanoparticles may be synthesized in water and are readily functionalized with biomolecules.

A magnetic particle may contain a magnetic material and one or more nonmagnetic materials, which may be a metal or a nonmetal. In certain embodiments of the invention the particle is a composite particle comprising an inner core or layer containing a first material and an outer layer or shell containing a second material, wherein at least one of the materials is magnetic. Optionally both of the materials are metals. In one embodiment the nanoparticle is an iron oxide nanoparticle, e.g., the particle has a core of iron oxide. Optionally the iron oxide is monocrystalline. In another embodiment the nanoparticle is a superparamagnetic iron oxide nanoparticle, e.g., the particle has a core of superparamagnetic iron oxide.

In certain embodiments of the invention the particle is a composite particle comprising an inner core or layer containing a magnetic material and an outer layer or shell containing a material such as gold or silver, so that the particle exhibits plasmon resonance (discussed below).

Methods for production of metal nanoparticles such as those described above are known in the art. Various suitable methods for making such particles and for attaching moieties thereto are found in U.S. Ser. No. 09/895,019 (U.S. Publication No. 20020086842), U.S. Ser. No. 10/165,258 (U.S. Publication No. 20030092029), and 16, 61-63.

In certain embodiments of the invention the nanoparticle is made at least in part of silica (SiO₂). For example, the particle may consist essentially of silica or may have an optional coating layer composed of a different material. In some embodiments the particle has a silica core and an outside layer composed of one or more other materials. In other embodiments the particle has an outer layer of silica and a core composed of one or more other materials. The other materials can be, e.g., any of a variety of metals or non-metals. The amount of silica in the particle, or in a core or coating layer comprising silica, can range from approximately 5% to 100% by mass, volume, or number of atoms, or can assume any integer value or range between 5 and 100%. Such values and ranges will not be set forth explicitly herein.

In certain embodiments of the invention the particle comprises a signal-generating moiety such as a quantum dot or fluorescent or luminescent protein or dye molecule. The signal-generating moiety can be entrapped within the core or surface layer of the particle or covalently or noncovalently linked to its surface or interior. The “surface” of a particle is understood herein to refer to the outer surface of a non-porous particle or, if the particle is porous, to the outer surface and to the surface of the pores or channels in the particle unless otherwise indicated or evident from the context.

Silica-containing nanoparticles may be made by a variety of methods. Certain of these methods utilize the Stober synthesis which involves hydrolysis of tetraethoxyorthosilicate (TEOS) catalyzed by ammonia in water/ethanol mixtures, or variations thereof (43). Microemulsion procedures can be used. For example, a water-in-oil emulsion in which water droplets are dispersed as nanosized liquid entities in a continuous domain of oil and sefactants and serve as nanoreactors for nanoparticle synthesis offer a convenient approach. Silica nanoparticles can be functionalized with biomolecules such as polypeptides and/or “doped” or “loaded” with certain inorganic or organic fluorescent dyes (see, e.g., U.S. Pub. No. 20040067503 and references 35, 44, 69-72).

In some embodiments of the invention the nanoparticle is made at least in part of a silica-polymer hybrid or nanocomposite. Such hybrids may be manufactured by the sol-gel process. Sol-gel technology is also a convenient way to produce nanoparticles that encapsulate a variety of different moieties in matrices such as silica, metallosilicates, metal oxides, and siloxanes, including biologically active moieties such as proteins. The method allows encapsulation of these molecules under conditions that allow retention of their activity (65-67).

In some embodiments of the invention a particle composed in part or essentially consisting of an organic polymer is used. A wide variety of organic polymers are known in the art. For example, particles composed at least in part of polymethylmethacrylate, polyacrylamide, poly(vinyl chloride), carboxylated poly(vinyl chloride), or poly(vinyl chloride-co-vinyl acetate-co-vinyl alcohol) may be used. Optionally the nanoparticle comprises one or more plasticizers or additives. Co-polymers, block co-polymers, and/or grafted co-polymers can be used.

Nanoparticles composed of any of the afore-mentioned materials may have a coating layer. Use of a biocompatible coating layer can be advantageous, e.g., if the particles contain materials that are toxic to cells. Suitable coating materials include, but are not limited to, proteins, polyethylene glycol (PEG) or a PEG derivative, phospholipid-(PEG), silica, lipids, carbohydrates such as dextran, etc. Coatings may be applied or assembled in a variety of ways such as by dipping, using a layer-by-layer technique, by self-assembly, etc. Self-assembly refers to a process of spontaneous assembly of a higher order structure that relies on the natural attraction of the components of the higher order structure (e.g., molecules) for each other. It typically occurs through random movements of the molecules and formation of bonds based on size, shape, composition or chemical properties. Self-assembly can be defined as the spontaneous organization of individual components into an ordered structure without human intervention.

As described elsewhere herein, many metal nanoparticles are made by a process that results in particles with a coating layer that serves to stabilize, solubilize, and/or prevent spontaneous aggregation of the particles under aqueous conditions, e.g., under physiological conditions. One or more additional coating layers may be applied to such particles.

In certain embodiments of the invention the particle is made at least in part of a porous material, by which is meant that the material contains many holes or channels, which are typically small compared with the size of the particle. For example the particle may be a porous silica nanoparticle, e.g., a mesoporous silica nanoparticle or may have a coating of mesoporous silica. Such particles have pores ranging in diameter from about 1 nm to about 50 nm in diameter, e.g., between about 1 and 20 nm in diameter. Between about 20 and 95% of the volume of the particle may consist of empty space within the pores or channels. In certain embodiments of the invention the pores are open, i.e., they communicate with the exterior of the particle. Methods for making mesoporous silica nanoparticles are known in the art (46 and references therein).

In general, it is frequently desirable to use a population of particles that is relatively uniform in terms of size and shape so that each particle has similar properties, e.g., similar optical or magnetic properties. For example, at least 80%, at least 90%, or at least 95% of the particles may have a diameter or longest straight line dimension that falls within 5%, 10%, or 20% of the average diameter or longest straight line dimension. In certain embodiments of the invention one or more substantially uniform populations of particles is used.

In certain embodiments of the invention the nanoparticle comprises a signal-generating moiety covalently or non-covalently attached thereto or encapsulated or entrapped therein. Exemplary signal-generating moieties include, but are not limited to, quantum dots and fluorescent or luminescent molecules. The signal can be generated by the emission of electromagnetic energy, e.g., in the form of UV, visible, or infrared light.

Fluorescence is a phenomenon in which absorption of light of a given wavelength by a fluorescent molecule is followed by the emission of light at longer wavelengths. The distribution of wavelength-dependent intensity that causes fluorescence is known as the fluorescence excitation spectrum, and the distribution of wavelength-dependent intensity of emitted energy is known as the fluorescence emission spectrum. See, e.g., Valeur, B., “Molecular Fluorescence: Principles and Applications”, John Wiley and Sons (2002), “Handbook of Fluorescent Probes and Research Products” (Molecular Probes, 9^{th} edition, 2002) and “The Handbook—A Guide to Fluorescent Probes and Labeling Technologies”, (Invitrogen, 10^{th} edition, available at the Invitrogen web site).

Chemiluminescence is the emission of light from a chemical reaction that occurs at or near ambient temperatures. Bioluminescence is a luminescent process mediated by an enzyme or other biological system. See, e.g., McCapra and Beheshti in “Bioluminescence and Chemiluminescence: Instruments and Applications”, K. Van Dyke (ed.), CRC Press, Boca Raton, Fla., pgs. 9-42 (1985).

Fluorescent and luminescent molecules include a variety of different organic or inorganic small molecules commonly referred to as “dyes” or “indicators”. Fluorescent and luminescent molecules also include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins (RFP, BFP, YFP, CYP, and SFP), reef coral fluorescent protein, etc. Luminescent proteins include luciferase, aequorin and derivatives thereof. Numerous fluorescent and luminescent dyes and proteins are described in the handbooks from Molecular Probes and Invitrogen mentioned above.

Quantum dots are nanocrystals with physical dimensions small enough (e.g., smaller than the exciton Bohr radius) such that the effect of quantum confinement gives rise to unique optical and electronic properties that are not observed either in the bulk material, in discrete atoms, or in larger nanoparticles. Semiconductor quantum dots are often composed of atoms from groups II-VI or III-V in the periodic table, but other compositions are also possible. Quantum dots generally have a broad absorption spectrum and a narrow emission spectrum. By varying their size and composition, the emission wavelength can be tuned (i.e., adjusted in a predictable and controllable manner) from the blue to the near infrared. Quantum dots and methods for their synthesis and conjugation with biomolecules are well known in the art (74 and references therein). Quantum dots with a large variety of absorption and emission spectra are commercially available, e.g., from Evident Technologies (Troy, N.Y.) or Quantum Dot Corp. (Hayward Calif.; now owned by Invitrogen), etc.

In certain embodiments of the invention the signal-generating moiety senses and produces a signal in response to one or more ions selected from the group consisting of Ca⁺⁺, Mg⁺⁺, Na⁺, K⁺, Cl⁻, H⁺, or Li⁺. In certain embodiments the moiety senses and produces a signal in response to an ion of an atom selected from the group consisting of rubidium, cesium, strontium, and optionally any of the foregoing ions. In some embodiments of the invention the sensing moiety is ion-selective, meaning that it selectively senses only one of the ions, e.g., the signal produced in response to the ion is at least 10-fold greater than the signal produced in response to the same concentration of one or more, or preferably any, of the other ions discussed herein.

In some embodiments of the invention the signal-generating moiety produces a signal in response to excitation by an external energy source. The signal may excite a second signal-generating moiety located in close proximity to the signal-generating moiety associated with the nanoparticle, e.g., by resonance energy transfer (RET), e.g., fluorescence resonance energy transfer (FRET), luminescence resonance energy transfer (LRET), or bioluminescence resonance energy transfer (BRET). Specific signal-generating moieties of use in different nanosensor components of the invention are discussed below.

The nanoparticles may possess more than one of the above properties or features. For example, a plasmon resonant nanoparticle or a magnetic nanoparticle of use in the invention can also comprise one or more signal-generating moieties. The invention thus provides multimodal nanoparticle sensor components comprising nanoparticles and a recognition domain. The multimodal nanoparticles are detectable by a plurality of different detection means.

The nanoparticle sensor components comprising a moiety such as a recognition domain, delivery moiety, and/or fluorescent or chemiluminescent moiety described herein may be produced using a variety of methods, some of which are described below and in the Examples. In some embodiments the recognition domain, delivery moiety, and/or fluorescent or chemiluminescent moiety is attached to a nanoparticle. The attachment may be to any portion of the nanoparticle. For example, certain nanoparticles comprise a coating or capping layer, and the moiety can be attached to such a layer. In the case of nanoparticle comprised at least in part of a porous material, the moiety can be attached to the surface of pores or channels. In general, attachment can be covalent or noncovalent.

In some embodiments a moiety such as a fluorescent or luminescent dye, quantum dot, etc., is entrapped, embedded, or encapsulated by a nanoparticle core and/or coating layer.

Preferably a recognition domain or delivery moiety is present at the surface of the nanoparticle so that it will be accessible to bind to the corresponding target domain or to interact with a cell to facilitate delivery, respectively.

A variety of methods known in the art can be used to attach a recognition domain, delivery moiety, and/or signal-generating moiety to a nanoparticle. Bifunctional crosslinking reagent can be employed. Such reagents contain two reactive groups, thereby providing a means of covalently linking two target groups. The reactive groups in a chemical crosslinking reagent typically belong to various classes of functional groups such as succinimidyl esters, maleimides, and pyridyldisulfides. Suitable cross-linking agents include, e.g., carboiimides, N-hydroxysuccinimidyl-4-azidosalicylic acid (NHS-ASA), dimethyl pimelimidate dihydrochloride (DMP), dimethylsuberimidate (DMS), 3,3′-dithiobispropionimidate (DTBP), etc.

Common schemes for forming a conjugate involve the indirect coupling of an amine group on one molecule to a thiol group on a second molecule, sometimes by a two- or three-step reaction sequence. A thiol-containing molecule may be reacted with an amine-containing molecule using a heterobifunctional crosslinking reagent, e.g., a reagent containing both a succinimidyl ester and either a maleimide, a pyridyldisulfide, or an iodoacetamide. Amine-carboxylic acid and thiol-carboxylic acid crosslinking may also be used.

Polypeptides can conveniently be attached to nanoparticles via amine or thiol groups in lysine or cysteine side chains respectively, or by an N-terminal amino group. If a polypeptide lacks a lysine or cysteine residue, a variant containing one or more of these residues, e.g., at the N- or C-terminus, can easily be prepared. Examples of methods by which polypeptides and other molecules can be linked to a nanoparticle or functionalized nanoparticle include maleimide-sulflhydryl coupling chemistries (e.g., the maleimidobenzoyl-N-hydroxysuccinimide ester (MBS) method). For example, in a specific embodiment MBS (an amine-to-thiol crosslinker, available from Pierce Biotechnology) is used to attach a coiled-coil peptide to a BSA-coated gold nanoparticle.

For additional information on conjugation methods and crosslinkers see generally the journal Bioconjugate Chemistry, published by the American Chemical Society, Columbus Ohio, PO Box 3337, Columbus, Ohio, 43210. See also “Cross-Linking”, Pierce Chemical Technical Library, available at the Web site having URL www.piercenet.com and originally published in the 1994-95 Pierce Catalog and references cited therein and Wong S S, Chemistry of Protein Conjugation and Crosslinking, CRC Press Publishers, Boca Raton, 1991; and G. T. Hermanson, Bioconjugate Techniques, Academic Press, Inc., San Diego, 1996.

It is to be understood that the compositions of the invention can be made in any suitable manner, and the invention is in no way limited to compositions that can be produced using the methods described herein. Selection of an appropriate method may require attention to the properties of the moieties being linked.

If desired, various methods may be used to separate nanoparticles with an attached polypeptide (or other moiety) from nanoparticles to which the polypeptide has not become attached, or to separate nanoparticles having different numbers of polypeptides attached thereto. For example, size exclusion chromatography or agarose gel electrophoresis can be used to separate populations of nanoparticles having different numbers of moieties attached thereto and/or to separate nanoparticles from other entities. A 3% agarose gel running in TAE buffer can be used to separate nanoparticles having an attached polypeptide from a population of nanoparticles. Other methods include size-exclusion or anion-exchange chromatography.

V. Sensor Designs

A. Sensors Utilizing Plasmon Resonant Nanoparticles

In some embodiments of the invention detection of ion channel activity or protein interaction utilizes the phenomenon of plasmon resonance to produce a detectable signal in response to the presence of an ion or in response to an interaction between two or more polypeptides such as a GPCR and a G protein subunit. In some embodiments of the invention the nanosensor comprises at least two nanosensor components, each of which comprises a plasmon resonant nanoparticle (described below). Each of the nanosensor components is attached to a cellular polypeptide such as an ion channel subunit. In other embodiments of the invention the nanosensor comprises a single plasmon resonant nanoparticle.

In the ion-sensing embodiments of the invention the plasmon resonant nanoparticle comprises a ligand-responsive binding moiety at its surface. The ligand-responsive moiety interacts with an ion of interest. An alteration in plasmon resonance properties occurs either as a result of an ion-induced alteration in the distance between the particles upon interaction of the ion with the binding moiety, or as a result of an ion-induced change in the refractive index of the local environment of the particle upon interaction of the ion with the binding moiety. An optical property of the particles indicative of the alteration in plasmon resonance properties is detected, thereby providing a signal in response to the ion.

In the embodiments of the invention directed to sensing protein interactions, an interaction between two or more polypeptides alters the distance between the polypeptides and thereby alters the distance between the particles. An alteration in plasmon resonance properties occurs as a result of the alteration in the distance between the particles attached to the polypeptides. An optical property of the particles indicative of the alteration in plasmon resonance properties is detected, thereby providing a signal that the polypeptides have interacted.

The phenomenon of plasmon resonance coupling between metal nanoparticles is well known in the art and is briefly described here to facilitate an understanding of the invention. Further discussion of plasmon resonance and other properties of metal nanoparticles is found in a review by Link and El-Sayed (19). When a nanometer-scale metal particle (usually made of a noble metal such as gold, silver, copper, platinum, etc.) is subjected to an external electric field, its conduction electrons are displaced from their equilibrium positions with respect to the nuclei, which in turn exert an attractive, restoring force. If the electric field is oscillating (as in the case of electromagnetic radiation such as light), the result is a collective oscillation of the conduction electrons in the particle (22). Such collective electron oscillation is called dipole plasmon resonance; higher modes of oscillation are also possible. As a result of the interaction between electromagnetic radiation and collective oscillation of conduction electrons, nanoparticles exhibit wavelength-selective absorption (usually in the visible range) characterized by large molar extinction coefficients and very efficient Rayleigh scattering.

The peak extinction wavelength (also referred to here as “peak plasmon resonance wavelength or “plasmon resonance wavelength”) of a nanoparticle's plasmon resonance spectrum (also referred to herein as “scattering spectrum” or “extinction spectrum”) depends on a number of factors, including: the material composition, the shape and size of the particle, the refractive index or dielectric properties of the surrounding medium, and the presence of other particles in the vicinity. If such factors change, the plasmon resonance spectrum of the nanoparticle also changes. According to certain embodiments of the invention metal nanoparticles are used to transduce chemical binding events into optically detectable signals. This is achieved in one of at least four ways: a) by monitoring changes in Rayleigh scattering, b) by monitoring changes that occur as a result of particle aggregation, c) by detecting binding of molecules or charge-transfer events occurring at the particle's surface and d) by detecting changes that occur as a result of alterations in the refractive index of the medium surrounding the particle. Events such as particle aggregation, binding of molecules to the particle surface, and alterations in refractive index induce shifts and/or broadening of the extinction and/or scattering spectrum of nanoparticles.

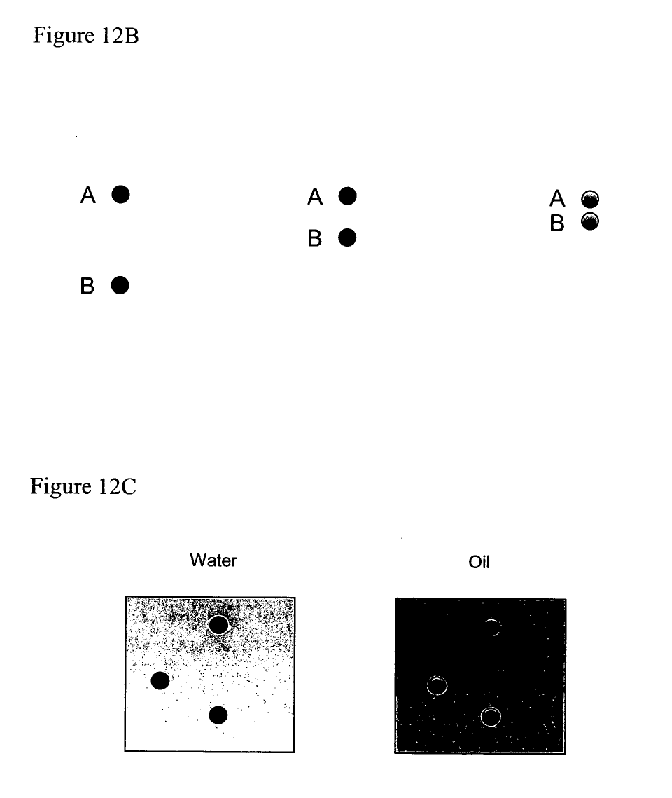

A particle that exhibits the phenomenon of plasmon resonance when excited with electromagnetic energy is referred to as a plasmon resonant particle. In general, spherical plasmon resonant particles of interest herein have a diameter of between approximately 2 nm and 200 nm, preferably between approximately 5 and 100 nm, more preferably between 10 and 50 nm. Selection of particular particle shapes, sizes, and compositions makes it possible to produce particles with a wide range of peak plasmon resonance frequencies, allowing for tuning of the sensors for detection of different ion concentrations and for concurrent detection of multiple ion species using particles with different properties. For example, as depicted schematically in FIG. 12A, the peak plasmon resonant wavelength of a spherical gold or silver nanoparticle increases, i.e., is “red-shifted” with increasing particle diameter.

The plasmon resonance phenomenon described above results in extremely efficient scattering and absorption of light by the particles over particular bands of frequencies. Scattering and absorption give rise to a number of distinctive optical properties that can be detected using various approaches including visually (i.e., by the naked eye or using appropriate microscopic techniques) and/or by obtaining a spectrum, e.g., a scattering spectrum, extinction (scattering+absorption) spectrum, or absorption spectrum from the particle(s). For example, a solution of gold nanoparticles (e.g., ˜15 nm diameter) appears red to the naked eye in transmitted light, while single gold nanoparticles of different diameters can appear green, red, blue, etc., under dark-field microscopy as a result of scattering. Single nanoparticles of sufficient size can be individually detected using a variety of approaches. For example, particles larger than circa 30 nm in diameter are readily detectable under an optical microscope operating in dark-field, and a spectrum from these particles can be obtained, e.g., using a CCD detector or other optical detection device. Despite their small dimensions relative to the wavelength of light, metal nanoparticles can be detected optically because they scatter light very efficiently at their plasmon resonance frequency. An 80 nm particle, for example, would be 5 million times brighter than a fluorescein molecule and 100,000 times brighter than a typical quantum dot under the same illumination conditions (23). In addition to dark-field microscopy, individual plasmon resonant particles, typically having diameters of 10 nm or greater, can be optically detected using a variety of other approaches including near-field scanning optical microscopy, differential interference microscopy with video enhancement, total internal reflection microscopy, photo-thermal interference contrast, etc.

Detection of ion channel activity or protein interaction is accomplished by detecting an optical property of the nanoparticle(s), which serves as a signal of ion channel activity or protein interaction. The optical property can be any spectral feature or optically detectable feature of a particle or population of particles, or a change in any such feature. Visually detectable features include, for example, color, apparent size, or simply whether or not the particle is visible under particular conditions (visibility). Spectral features associated with light scattering and/or absorption by the particles include, for example, peak resonant wavelength or frequency (wavelength or frequency at which maximum scattering intensity, extinction, or absorption occurs), peak magnitude (e.g., peak scattering intensity, peak absorbance value), peak width at half height, or metrics derived from any of the foregoing such as ratio of peak magnitude to peak width. Certain spectra may contain multiple peaks, of which one is typically the major peak and has significantly greater intensity than the others. Each of these peaks has associated features. Unless otherwise indicated, for any particular spectrum, spectral features such as peak wavelength or frequency, peak magnitude, peak width at half height, etc., are generally determined with reference to the major peak. The features of each peak, number of peaks, separation between peaks, etc., can also be considered to be features of the spectrum as a whole. The foregoing features can also be measured as a function of the direction of polarization of light illuminating the particles; thus polarization dependence can be measured. Features associated with hyper-Rayleigh scattering can also be measured. Optical properties of metal nanoparticles have been recently reviewed by Link and El-Sayed (19).

Spectral features can be detected using a variety of approaches. For example, an extinction spectrum from a population of nanoparticles (e.g., a population of particles in solution, on a substrate, etc.) is easily obtained by means of a spectrophotometer, e.g., a UV/Visible spectrophotometer such as a Beckman-Coulter DU-800. Such spectra are frequently referred to as absorption spectra, though what is measured is typically extinction (absorption+scattering). U.S. Ser. No. 09/740,615 describes methods for detecting a number of different spectral features associated with scattering by individual nanoparticles or populations of nanoparticles.

Plasmon resonant particles typically exhibit significant scattering and/or absorption in the optical region of the electromagnetic spectrum, rendering them optically detectable. The optical region of the spectrum is that portion of the spectrum extending from approximately 180 nm to several microns. In certain embodiments of the invention a plasmon resonant particle exhibits significant scattering and/or absorption within the visible portion of the electromagnetic spectrum, i.e., the portion of the spectrum that is detectable by the human eye (approximately 400 nm to approximately 700 nm). For example, the plasmon resonance wavelength of an isolated nanoparticle, or the shifted plasmon resonance wavelength exhibited by aggregated nanoparticles, may fall within the visible region.

In certain embodiments of the invention a plasmon resonant particle is selected or modified so as to exhibit significant scattering and/or absorption within the infrared portion of the electromagnetic spectrum. For example, the plasmon resonance wavelength of an isolated nanoparticle, or the shifted plasmon resonance wavelength exhibited by aggregated nanoparticles, may fall within the infrared region of the spectrum, which extends between approximately 700 nm and 0.01 cm. (Plasmon resonance frequencies are considerably less than 0.01 cm.) Plasmon resonant nanorods have a plasmon resonance wavelength that can be tuned to and within the infrared region, which can be tuned by altering the aspect ratio (length to width) of the nanorod. Plasmon resonant nanoshells, e.g., gold or silver nanoshells, also have a plasmon resonance that can be tuned to and within the infrared region. Infrared absorption spectroscopy, or other infrared detection methods, may be used to detect optical features of such particles.

The plasmon resonance frequency and optical properties of metal nanoparticles are sensitive to their local environment. In particular, when two nanoparticles are in close proximity to one another (separated by a distance comparable to their diameter or short-axis length), the oscillations of their electrons become coupled, resulting in a marked red-shift and broadening of their spectrum (24, 25). For example, in a macroscopic, colloidal dispersion of gold nanoparticles in water the nanoparticles remain in solution at least in part because of their mutual electrostatic repulsion. If conditions change so as to reduce such repulsive forces (for example due to increased ionic strength), particles tend to aggregate. Under such conditions the oscillations of their electrons become coupled and their absorption spectrum changes (FIG. 14). The color of the solution also changes from red to purple (for gold particles). By simple visual inspection of the solution or detection of a change in one or more spectral features, such as a shift in peak wavelength of the absorbance, events happening at the nanometer-scale level can be detected. Changes in spectral features, e.g., shifts in peak wavelength, appearance of additional peaks, peak broadening, etc., can be detected when the separation between particles becomes less than approximately 2.5 particle diameters or short-axis length, though generally a separation of approximately 1 diameter or short-axis length, or less, is preferred for detection of such changes.

The color of a metal nanoparticle, e.g., under dark-field microscopy, corresponds to the frequency of its peak plasmon resonance. Therefore aggregation of individual nanoparticles causes a visually detectable color change and a change in spectral features such as peak scattering wavelength. For example, the color of particles under dark-field may change from green to yellow or orange-red when two particles are brought into proximity with one another (e.g., to within a distance of 1 diameter). For example, as depicted schematically in FIG. 12B silver nanoparticles (labeled A and B, with typical peak plasmon resonance around 450 nm) would appear blue-green under dark-field microscopy. When two such particles are brought into close proximity, the movements of their free electrons become coupled and their peak plasmon resonance red-shifts. Such particles would then appear yellow under dark field microscopy (the resolution of optical microscopy does not allow to distinguish two nanoparticles in such close proximity; therefore the two would appear as a single yellow spot). This effect has been demonstrated in an experiment in which the position of single silver nanoparticles was controlled by means of optical tweezers (26). Nanoparticles made of other metals, e.g., gold or composite particles, behave in a similar manner.

A variety of other spectral features that change when particles are brought into proximity with one another may be measured in addition to, or instead of, peak scattering wavelength. For example, peak intensity, peak width at half peak height, or any other proximity-dependent spectral feature or combination thereof can be measured. A spectral feature of a nanoparticle or population of nanoparticles that exhibits a change when particles are brought into proximity with one another is referred to herein as a proximity-dependent spectral feature.

The present invention provides a variety of nanoscale sensors that utilize an optical property of one or more plasmon resonant particles to detect ion channel activity or protein interaction. In one embodiment, the invention provides a ligand-responsive nanoparticle sensor component. The nanosensor component comprises a nanoparticle that comprises a ligand-responsive binding moiety. The binding moiety is located at the surface of the particle so that it is available to bind to a ligand such as an ion or other analyte. For example, the particle may be coated with a binding moiety that binds to an ion of interest. If the particle is porous, the binding moiety can be attached to the surface of the pores or channels.

In the context of a ligand-responsive nanosensor component, the term “ligand” means any molecular entity (e.g., an ion or other molecular analyte, or a unit composed of multiple molecules) that binds to another molecular entity or entities or portion(s) thereof, which is termed a “binding moiety”. For purposes of the present invention in the context of a ligand-responsive nanosensor component, the term “ligand” will generally refers to an analyte such as an ion, whose detection is desired, while the term “binding moiety” will generally refer to a moiety that is part of or attached to a nanosensor component and that either by itself or in combination with another moiety that is part of or is attached to either the same or a different nanosensor component, binds to an analyte. The binding interaction results in a physical association such as formation of a complex (e.g., a coordination complex) between the ligand and the binding moiety, chelation of the ligand by one or more binding moieties, noncovalent binding (e.g., as a result of van der Waals forces, hydrogen bonds, electrostatic forces) of the ligand and one or more binding moieties, etc. In general, the binding interaction between a ligand and one or more binding moieties will not be via covalent binding, although this possibility is not excluded. The interaction between a binding moiety and a ligand can be reversible or irreversible.

When referring to ligands and binding moieties, or to interactions between them, it is to be understood that the reference generally applies to individual binding moiety/ligand interactions, to interactions between multiple binding moieties and multiple ligands, or both. The term “ligand” can thus refer either to a single ligand or a plurality of ligands of that molecular species, and the term “binding moiety” can refer either to a single binding moiety or to a plurality of binding moieties of that species.

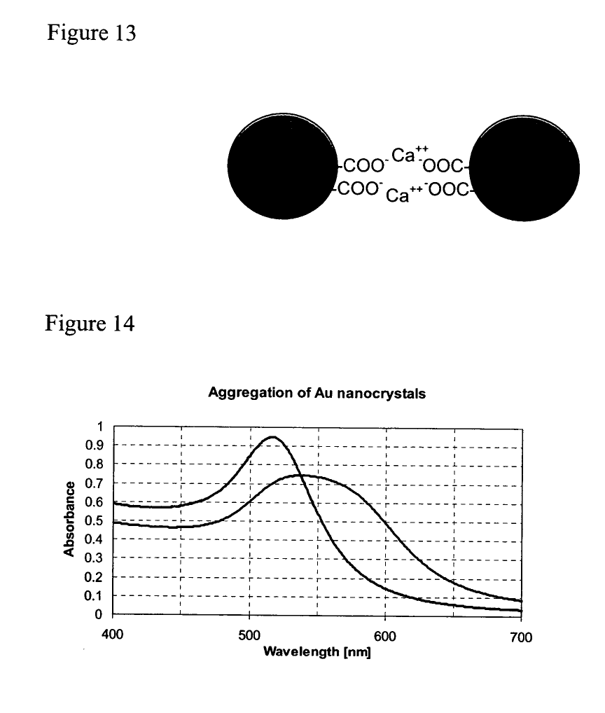

A binding moiety that interacts with a particular ligand is said to be “responsive to” or “sensitive to” that ligand. Preferably the interaction should be (i) sufficient to cause a change in a property of the binding moiety (e.g., a structural rearrangement of atoms; a conformational change in which the spatial relationship of two or more atoms to one another is altered; a change in refractive index or dielectric constant, which may be associated with a structural or conformation alteration, etc.), and/or (ii) sufficient to neutralize at least in part the charge of the binding moiety, and/or (iii) sufficient to overcome repulsive or attractive forces between two or more binding moieties; and/or (iv) sufficient to produce a bridge between two or more binding moieties or portions thereof, in which the ligand serves as the bridge (as depicted schematically in FIG. 13). In certain embodiments of the invention an interaction between a ligand and one or more binding moietie(s) attached either to a single nanoparticle sensor component (e.g., a nanoparticle) or between a ligand and binding moietie(s) attached to different nanoparticle sensor components (e.g., two binding moieties, each of which is attached to a different nanoparticle) is sufficient to cause a change in distance between the particles. Presence of a ligand-responsive binding moiety at a surface of a nanoparticle sensor component renders the nanoparticle sensor component responsive to the ligand. The distance between two particles (or other components) can be measured with respect to any fixed points of the particles (or components), e.g., between the centers of two spherical particles, the centers of mass of two irregularly shaped particles or components, etc. In general, the physical association of two or more molecular entities (e.g. a binding moieties and a ligand), or a resulting change in distance between two sensor components to which the binding moieties are attached, should be stably maintained under the conditions in which it occurs (e.g., under physiological conditions) for a period of time sufficient for detection, e.g., using the various optical detection means described herein.

In certain embodiments of the invention, interaction between (i) binding moieties attached to nanosensor components and (ii) their corresponding ligands causes a physical association between the sensor components. The term “aggregation” is used herein to refer to a physical association of nanoparticles. The physical association may be irreversible or may be reversible, e.g., when conditions such as local ion concentration return to a state similar to that which existed before the association occurred. The overall interaction between binding moieties attached to nanoparticles and ligands that interact with such binding moieties typically involves many individual ligand molecules and many individual binding moieties. The binding moieties may be attached to a single nanoparticle, or individual binding moieties may be attached to different nanoparticles. Aggregation of two or more nanoparticles, e.g., as a result of the presence of a ligand, or an increase in its concentration, results in a change in the distance between them, thereby altering their plasmon resonance properties.

In certain embodiments of the invention, rather than causing an association between binding moieties, the presence of a ligand results in dissociation of two or more binding moieties that are associated with each other (optionally via association with a ligand). For example, if binding moieties having positively and negatively charged groups are associated via electrostatic forces, presence of a positively or negatively charged ion may cause dissociation. For example, the presence of ions that have a greater positive charge than that of the positively charged binding moiety may cause dissociation. Nanoparticles to which the binding moieties are attached would then disaggregate, causing a shift in plasmon resonance frequency and optically detectable changes in color, spectral features, etc.

In order to detect a specific ion, the nanoparticles are provided with a binding moiety that interacts with that ion. In certain embodiments of the invention a sensor that detects activity of an ion channel comprises a binding moiety that has the opposite charge to that of the ion to be detected, or which becomes ionized such that it possesses such a charge under physiological conditions. Thus binding moieties for positively charged ions (cations) such as Ca⁺⁺ include, but are not limited to, negatively charged (anionic) groups such as ionized carboxyl, phosphate, or sulfate groups, etc., or moieties that become ionized at physiological pH to yield such negatively charged groups. Binding moieties for negatively charged ions, include, but are not limited to, positively charged groups such as protonated amines, or moieties that become ionized at physiological pH to yield such positively charged groups.

In an embodiment of particular interest the ion is Ca⁺⁺. A variety of different binding moieties that interact with calcium ions can be used. In an exemplary embodiment the binding moiety comprises one or more carboxyl groups (COOH), which ionizes to provide COO⁻ groups under physiological conditions. In general, suitable molecules have the structure R(COOH), where R represents any of a wide variety of carbon-containing moieties such as aliphatic, alicyclic, heteroaliphatic, heteroalicyclic, aromatic, or heteroaromatic moieties, which can be linear or branched and can be substituted or unsubstituted, and wherein the molecule ionizes to provide COO⁻ groups. In certain embodiments the number of carbon atoms in R is between 1 and 30, e.g., between 6 and 14. In certain embodiments of the invention R is a substituted or unsubstituted aliphatic or heteroaliphatic chain having a length of 5 or fewer carbon atoms, e.g., 1, 2, 3, 4, or 5 carbon atoms.

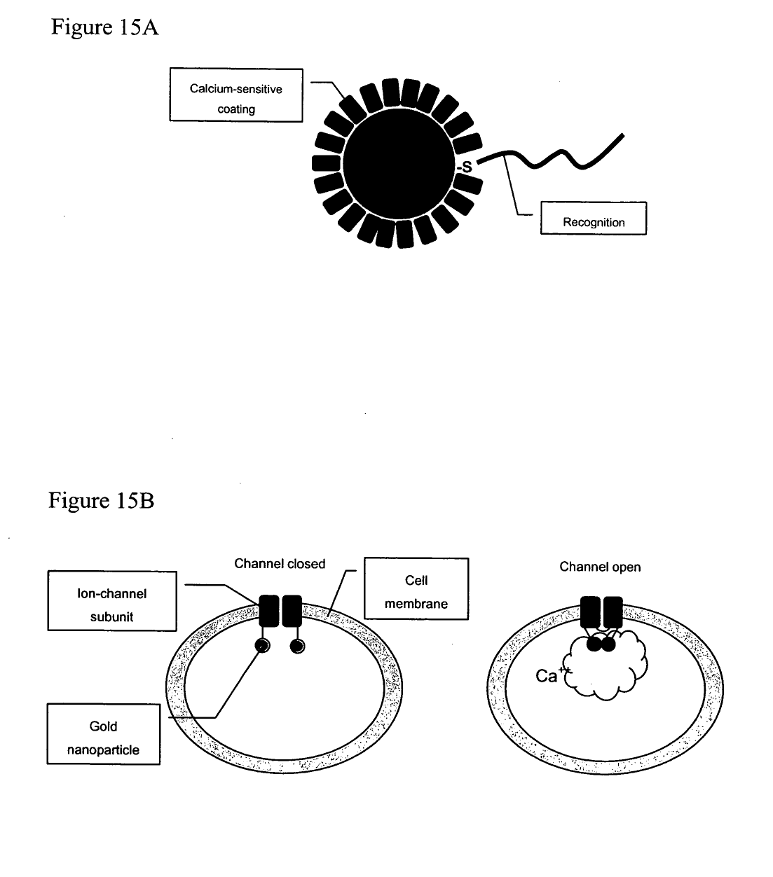

FIG. 13 is a schematic representation of the calcium-induced aggregation of nanoparticles coated with a binding moiety that provides COO⁻ groups. The particles remain separated from one another as a result of electrostatic repulsion. When the concentration of calcium in solution increases beyond a certain threshold, the particles come into proximity with one another, which is an optically detectable phenomenon. Without wishing to be bound by any theory, particle aggregation may occur as a result of the propensity of the negatively charged groups to trap positively charged Ca⁺⁺ ions in between them, thereby overcoming the particles' electrostatic repulsion. Ca⁺⁺ ions may be chelated by neighboring COO⁻ groups on the same particle, which would neutralize the particle's charge, leading to aggregation. FIG. 14 shows the change in absorption spectrum that occurs upon addition of Ca⁺⁺ to a solution containing gold nanoparticles whose surface was functionalized with carboxylate groups. FIG. 15A shows a schematic representation of a gold nanoparticle functionalized for calcium sensitivity.

The invention provides nanoscale sensors that utilize one or more ion-sensitive nanosensor components. In a first embodiment the nanoscale sensor comprises at least two nanosensor components. Each of the nanoparticle sensor component comprises a recognition domain that binds to a cellular polypeptide of interest that comprises a corresponding target domain. The recognition domains may be identical or different and may bind to either the same target domain or to different target domains in various embodiments of the invention.

The operation of a sensor that detects ion channel activity is depicted schematically in FIG. 15B. The nanoparticle sensor components are introduced into a cell that expresses subunits of an ion channel of interest, in this case a calcium channel. The ion channel can consist of an ion channel subunit that comprises two target domains or the ion channel can be composed of at least two ion channel subunits, each of which comprises a target domain, as depicted in FIG. 15B. The nanosensor components attach to the ion channel subunit(s) such that at least two nanosensor components are attached to the ion channel (either to a single subunit or to different subunits). Attachment may occur at any time after synthesis of the ion channel subunit. Ion channel activity results in passage of an ion through the channel pore. In a non-limiting example, upon channel opening, a sudden burst of calcium enters the cell through the channel, raising the ion's concentration approximately 1,000-fold near the channel's mouth. The baseline calcium concentration in the cytosol is approximately 100 nM; after channel opening the concentration is estimated to increase to circa 1 mM near its mouth. The sharp rise in calcium concentration remains a local phenomenon at least in part because calcium ions are rapidly buffered by various calcium binding proteins as they diffuse outward into the cytosol.

The increase in calcium concentration upon channel opening is sufficient to cause aggregation of the nanoparticles and a red-shift in their plasmon resonance wavelength. The shift appears under dark-field as a change in particle color, e.g., from green to orange-red, and results in corresponding changes in the scattering spectrum, e.g., a shift in peak plasmon resonance wavelength. If a sufficient number of channel opening events occurs, as may take place in a population of cells containing nanosensor components attached to ion channel subunits (e.g., in the presence of a stimulus that causes such opening,), then the shift in plasmon resonance wavelength can be detected using an absorption spectrometer. Alternately, channel opening events (either in an individual cell or in a plurality of cells), can be optically detected using microscopy-based approaches. For purposes of simplicity, FIG. 15B and other figures herein depict a channel as having two subunits, each with a nanosensor component attached thereto. However, it is to be understood that the channel may contain only a single subunit or may contain more than two subunits, and that any of these subunits may have one or more nanosensor components attached thereto.

A variety of different binding moieties can be employed for the detection of an ion of interest. Methods for synthesizing metal nanoparticles typically make use of a variety of so-called “capping agents” to render the particles stable, water-soluble, and/or to prevent their spontaneous aggregation. Capping agents include various alkanethiols, citrate, etc. Certain of these capping agents provide negatively charged groups that may render the nanoparticles ion-responsive and are of use in certain embodiments of the invention. However, in other embodiments of the invention the capping agent is not an alkanethiol or citrate.

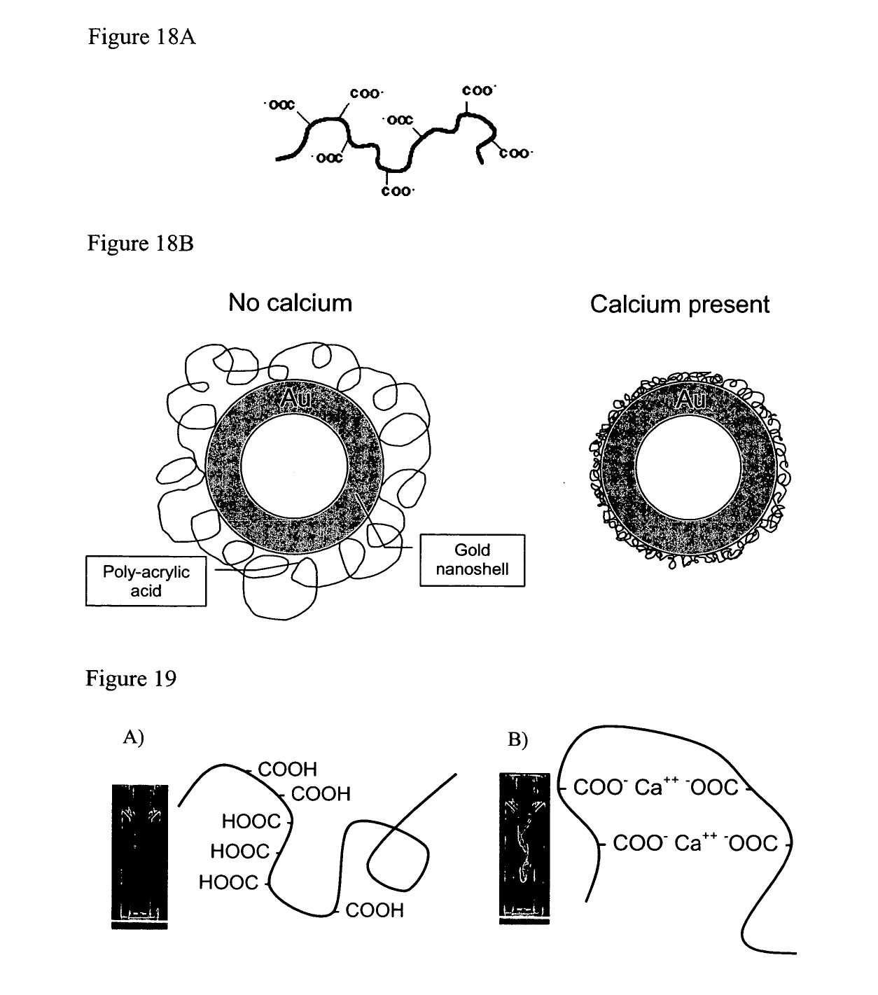

In certain embodiments of the invention the binding moiety is a polymer composed of monomeric subunits having the structure R(COOH) or R(COO⁻) where R is between 1 and 30, e.g., R contains 1, 2, 3, 4, or 5 carbon atoms. Any polymer that exists as a polyanion under physiological conditions can be used. FIG. 18A shows the general structure of a polyanion. Polyacrylic acid, which ionizes to give polyacrylate, is an exemplary polymer of use in the invention for detecting a positively charged ion such as calcium. Polymers such as polyacrylic acid comprising a plurality of negatively charged groups when ionized are preferred in certain embodiments of the invention as they provide a convenient means of providing multiple negatively charged groups at the surface of a nanoparticle while requiring only one point of direct attachment to the particle itself.

In certain embodiments of the invention the binding moiety is a peptide. Nanoparticles with a particularly high stability and solubility in aqueous media can be produced by attaching any of a variety of different peptides (or combinations thereof), to the metal surface as capping agents (28). Suitable peptides include, for example, peptides having sequences CALNN (SEQ ID NO: 13), CTTTT (SEQ ID NO: 14), CCALNN (SEQ ID NO: 15), CCVVVT (SEQ ID NO: 16), etc. In certain embodiments the peptide is at least 5 amino acids in length, e.g., between 5 and 10, or between 5 and 20 amino acids in length. In certain embodiments the peptide has a terminal cysteine residue, e.g., at the N-terminus, or two cysteines at the N-terminus. Other features typically include the presence of hydrophobic amino acids at the second and third positions following the one or two cysteines. Optionally the peptide is amidated at its C-terminus. Additional suitable capping peptides may be designed using the principles described by Fernig and coworkers (28). The peptides may interact with a gold surface via the thiol group in the side chain of an N-terminal cysteine or via the amino group in the N-terminal primary amine.

Attachment of other polypeptides (e.g., delivery-enhancing moieties, polypeptides comprising recognition domains, etc.) to the particle surface can be achieved simply by mixing the desired polypeptides with the capping peptides or by utilizing capping peptides containing at least two domains, one of which provides the desired stability and the other of which comprises a delivery-enhancing peptide or recognition domain. Peptides having a terminal lysine residue, e.g., CALNNGK (SEQ ID NO: 17), can be included in the mixture (e.g., at a relatively low concentration such as 1% to several percent). The lysine contains an amino group that provides a convenient site for conjugation of additional moieties, e.g., using any of a variety of commercially available crosslinkers such as MBS (Pierce Biotechnology). Alternately, additional moieties such as polypeptides comprising a recognition domain may be conjugated to nanoparticles (e.g., via a terminal cysteine) prior to coating the nanoparticles with the capping peptide.

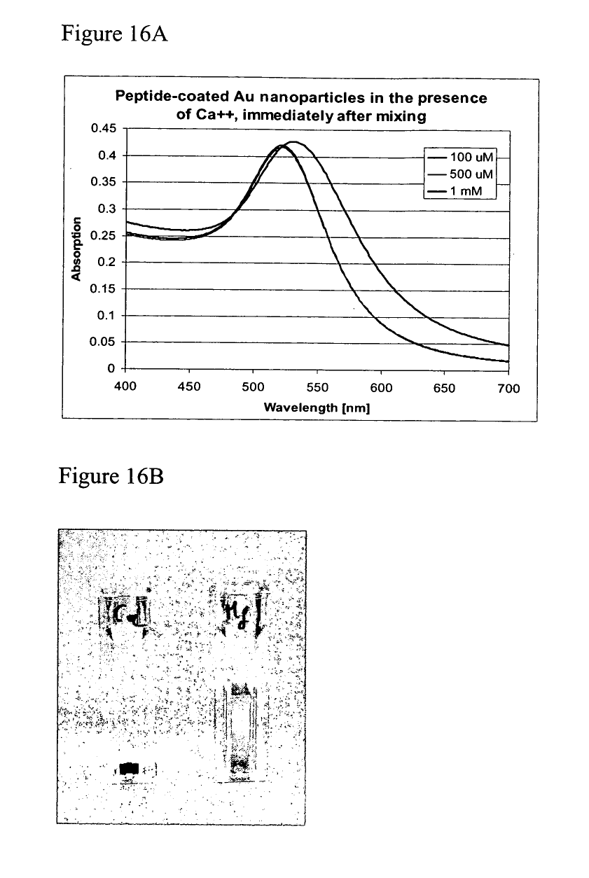

Asparagine is found very frequently in calcium-binding motifs in naturally occurring proteins (27). Without wishing to be bound by any theory, peptides that comprise a plurality of asparagine residues are likely to display increased affinity for Ca⁺⁺ relative to their affinity for other cations such as Mg⁺⁺. In certain embodiments of the invention a capping peptide comprising a plurality of asparagine residues is used. For example, the peptide may comprise between 20% and 100% asparagine residues, e.g., at least 30%, at least 40%, at least 50%, at least 60%, at least 70%, at least 80%, or at least 90% asparagine residues. In certain embodiments of the invention the peptide contains 1, 2, or 3 C-terminal asparagine residues. FIGS. 16A and 16B show that plasmon resonant nanoparticles coated with a peptide comprising a plurality of asparagine residues undergo aggregation in the presence of 1 mM Ca⁺⁺ and undergo a change in their absorption spectrum. No aggregation or change in absorption spectrum of these particles occurred in the presence of 1 mM Mg⁺⁺, indicating that these particles are selectively responsive to Ca⁺⁺.

Sensitivity to a particular ion such as Ca⁺⁺ can be tuned by changing the particles' diameter and/or the molecular coating. For example, since van der Waals attraction forces between metal nanoparticles increase with their diameter, the threshold for particle aggregation can be lowered (and the sensitivity to calcium increased) by using larger particles with a given coating. Selectivity for different ions, e.g., Ca⁺⁺ versus Mg⁺⁺, can also be tuned by appropriate selection of particle size, composition, etc., and by the choice of the coating molecules.

Sensors for detection of any of a variety of analytes may be created based on the principles described above by selection of appropriate binding moieties. In certain embodiments of the invention the binding moiety comprises a cyclic polyether referred to as a crown ether (29, 30). Structures of three typical crown ethers are given below. The common names of these ethers include a number as a prefix to designate the total number of atoms in the ring and a number as a suffix to designate the number of oxygen atoms in the ring. Thus, 15-crown-5 is comprised of 15 atoms in the ring, 5 of which are 0 and 10 of which are C.

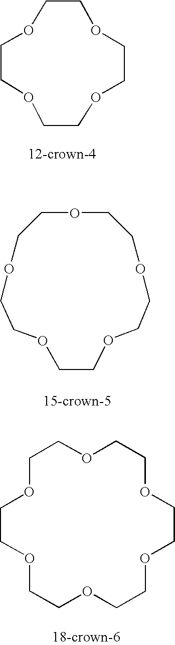

The characteristic chemistry of crown ethers involves complexation of the ether oxygens with various cationic species, including Ca⁺⁺, Mg⁺⁺, Na⁺, K⁺, Li⁺, etc. This is often termed “host-guest” chemistry, with the ether as host and the ionic species as guest (31). This type of chemistry (host-guest) is also found in a variety of other contexts, e.g., in cyclodextrins and macrocyclic polyether antibiotics. Certain crown ethers display selective complexation with one or more cations. Crown ethers can be attached to nanoparticles using a number of different approaches, e.g., through use of crown ether tagged alkanethiols (32). Thus crown ethers, or compounds containing crown ethers, can be used to provide binding moieties useful for detecting the activity of a wide variety of channels that permit passage of one or more of the ion species with which the crown ether binds.

Zeolite crystals can be fabricated to bind specifically to certain ions, e.g., potassium or sodium ions and are of use in the invention. The activity of potassium and sodium channels is optically detected by exploiting the particles' plasmon resonance sensitivity to ions adsorbed onto their surface (bound to zeolites in this case).

The invention encompasses use of multiple different binding moieties in combination, each of which interacts with a ligand or ligands of interest.

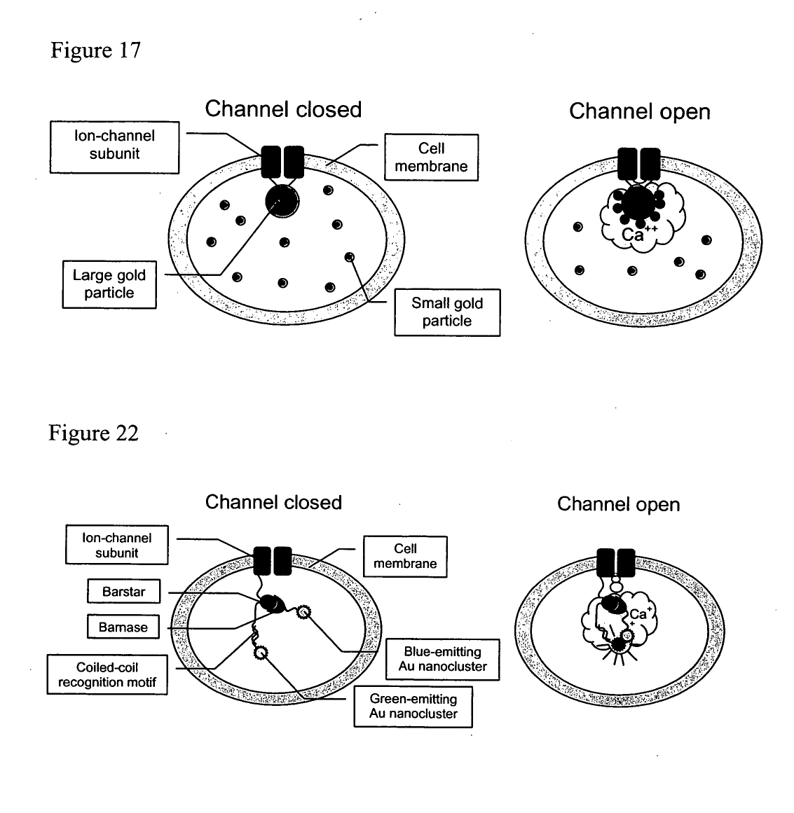

A second embodiment of an ion-sensitive nanoscale sensor is shown in FIG. 17. As shown therein, a nanosensor component comprising a nanoparticle with an ion-responsive binding moiety at its surface is introduced into a cell and attaches to an ion channel subunit. A plurality of small metal nanoparticles that are also modified for ion sensitivity by attachment of a suitable binding moiety to their surface is also introduced into the cell. The smaller particles may be optically detectable or may be too small to be detected individually under dark-field microscopy. In certain embodiments the nanoparticles have a diameter between 1 and 10 nm. The cell is typically loaded with particles ranging in number from tens to several hundred, several thousand, several hundred thousand or more. Upon channel opening and entry of an ion such as Ca⁺⁺, the smaller particles aggregate onto the larger one, causing a shift in its plasmon resonance properties and a change in its color under dark-field microscopy. Aggregation can be detected visually or by measuring a spectral feature of a scattering, extinction, or absorption spectrum.

One of the factors influencing the plasmon resonance frequency of metal nanoparticles is the refractive index of their surrounding medium (23). As a result, the color of a given nanoparticle, e.g., when viewed under dark-field microscopy, depends in part on the surrounding environment and can be changed by changing aspects of that environment. For example, the peak plasmon resonance shifts to longer wavelengths upon increasing the solvent refractive index or dielectric constant of the surrounding material. For example, as shown schematically in FIG. 12C, if nanoparticles (seen as green spots under dark-field microscopy) were to be transferred from air to oil, they would be seen as yellow in the new medium; removing the oil would shift the colors back to the original (82). Since the refractive index and dielectric constant are related, for purposes of simplicity the description herein will refer to the refractive index. As a consequence of this refractive index sensitivity, the plasmon resonance properties of metal nanoparticles also changes when molecules adsorb onto their surface; indeed, this is equivalent to changing the local refractive index of their surrounding medium (22, 82, 83).

A third embodiment of an ion-sensitive nanoscale sensor utilizes a nanosensor component comprising a plasmon resonant nanoparticle comprising a coating layer that undergoes a change in refractive index in the presence of an ion. For example, the coating layer comprises a material that undergoes a conformational change in response to an analyte such as an ion. A “conformational change” in a molecule refers to a rearrangement of the position of atoms with respect to one another. The conformational change alters the refractive index of the coating layer. A variety of materials that undergo an ion-responsive conformational change can be used. For example, when dissolved in water, polymers such as polyacrylic acid acquire a random-coil conformation but “collapse” promptly upon encounter with calcium ions (40) and precipitate out of solution. Formation of intermolecular bridges between COO⁻ groups in a similar manner to that described above results in shrinkage of the coils.

FIG. 18B shows a schematic diagram of a hollow nanoparticle (nanoshell) comprising a polymer coating that undergoes a conformational change in the presence of Ca⁺⁺. In the absence of Ca⁺⁺ (left) the coils of the polymer assume an open, extended configuration. Upon the addition of Ca⁺⁺ (right) the coils assume a closed, collapsed configuration resulting in a change in the refractive index of the coating and a change in the plasmon resonance properties of the particle. In order to detect calcium channel activity, a nanosensor component comprising a coated nanoparticle is attached to an ion channel subunit as described above. An influx of Ca⁺⁺ causes collapse of the coating and a detectable change in plasmon resonance properties.

Plasmon resonant particles coated with PAA show a plasmon resonance wavelength shift of circa 5 nm in the presence of 2 mM calcium, which is reversible upon removal of the ion (FIGS. 19A and 19B). The polyacrylate concentration at which polymer collapse occurs correlates with the local concentration of calcium (40). Therefore, sensors based on this design can be tuned to detect a range of calcium concentrations.

A variety of different coating materials that alter their conformation in the presence of an ion can be used in addition to, or instead of, PAA, including other polymers that are polyanions under physiological conditions. Polymers comprising crown ethers aggregate in the presence of ligands that form complexes with the crown ether (89), likely resulting in a change in their refractive index. Polypeptides that respond to the presence of calcium by altering their conformation are also of use. Examples include calmodulin and myosin light chain kinase (MLCK). For example, MLCK is known to contract in the presence of calcium ions. Particles coated with MLCK or calmodulin exhibit a change in plasmon resonance properties upon calcium-induced change in conformation. Specific detection of particular ions is achieved by choice of a coating material that undergoes a conformational change in response to the desired ion.

The sensitivity of the plasmon resonance to local refractive index depends on the chemical composition and shape of the particle, and both of these parameters can be varied to achieve a desired specificity and/or sensitivity. For example, silver particles are more sensitive to refractive index changes than gold ones; moreover, silver triangular particles are more sensitive than spherical ones (9). Gold nanoshells are also much more sensitive to such environmental changes than solid gold nanospheres (10). Thus in certain embodiments of the invention silver nanoparticles or gold nanoshells are used in a sensor that employs a change in refractive index for ion detection.

In certain embodiments of the invention two nanoparticles are joined to one another by a polypeptide that alters conformation in the presence of calcium such as MLCK. One of the nanoparticles is also attached to a target domain of a polypeptide of interest such as an ion channel subunit. Passage of calcium through the ion channel increases the local calcium concentration, causing the linking polypeptide to alter its conformation. The conformational change brings the nanoparticles into proximity with one another, thereby detectably altering their plasmon resonance properties.

Nanoparticle sensor components comprising plasmon resonant particles are used for detection of protein interactions in various embodiments of the invention. Nanosensor components, each comprising a plasmon resonant nanoparticle and a recognition domain, are attached to first and second polypeptides (e.g., a GPCR and a G protein subunit). Interaction of the polypeptides brings the particles into close proximity, detectably altering their plasmon resonance properties. The change in an optical property of the particles, e.g., their plasmon resonance frequency, is detected as described above and indicates polypeptide interaction. For example, activation of a GPCR results in engagement with G protein subunits, bringing them into closer proximity. Alternately, dissociation of polypeptides (e.g., dissociation of a GPCR from a G protein subunit or dissociation of G protein subunits from one another) results in an increased distance between nanoparticles attached to the respective polypeptides, thereby altering their plasmon resonance frequency.

B. Sensors Utilizing Magnetic Nanoparticles

Other embodiments of the invention employ nanosensor components that comprise magnetic nanoparticles. In a first embodiment the magnetic nanoparticles are coated with a ligand-responsive binding moiety and are attached to one or more ion channel subunits via binding of specific recognition and target domains as described above. Flux of an ion through an ion channel results in an alteration in the proximity of the magnetic particles essentially as described above for plasmon resonant particles. A property of the system that depends on the proximity of the magnetic nanoparticles to one another is detected.

Any of a variety of proximity-dependent properties can be detected. For example, in certain embodiments of the invention a spontaneous change in properties of magnetic particles occurs when the particles come into close proximity to one another. While not wishing to be bound by any theory, magnetic nanoparticles that are in close proximity to one another may become more efficient at dephasing the spins of surrounding water protons, enhancing spin-spin relaxation times (T2) (73). As described above, nanoparticle aggregation occurs during ion channel activity when nanoparticles are attached to an ion channel. Nanoparticle aggregation results in a change in the relaxation time of water. Such a change can be detected in a variety of ways. For example, a handheld nuclear magnetic resonance (NMR) relaxometer can be used. Alternately, a magnetic resonance image can be obtained. MRI is conveniently employed to determine T2 relaxation times from a plurality of samples in a multi-well plate, providing a convenient means for high throughput screening. High throughput NMR and MR spectrometers are known in the art and could also be used.

In another embodiment the nanoparticles are composed of appropriate materials such that the particles are not individually magnetic but become so when aggregated. Certain materials having such properties are known in the art. The particles are functionalized with a ligand-sensitive binding moiety as described above. Ligand-induced aggregation upon the opening of an ion channel to which such particles are attached creates a nano-magnet that can be detected by means that do not rely on optical microscopy or spectroscopy. In addition, the ligand-induced magnetization allows a magnetic extraction, out of a population of cells, of only of those cells whose channels of interest are active.

Any suitable method can be used for detecting magnetic properties, water relaxation times, and/or changes therein. For example, detectors based on the phenomenon of magnetic resonance can be employed. Superconducting quantum interference devices (SQUID), which use the properties of electron-pair wave coherence and Josephson junctions to detect very small magnetic fields can also be used. Magnetic force microscopy or handheld magnetic readers can also be used. U.S. Ser. No. 10/165,259 (Publication No. US2003/009029) describes various suitable methods.

Similar methods can also be used to efficiently detect intracellular interaction of polypeptides such as GPCRs and G protein subunits comprising the target domains described herein and having nanosensor components comprising magnetic nanoparticles attached thereto via corresponding recognition domains.

C. Sensors Utilizing Resonance Energy Transfer

In additional embodiments of the invention the nanoscale sensors utilize the phenomenon of resonance energy transfer to produce a signal in response to ion channel activity or protein interaction. Certain nanosensor components of the invention comprise a resonance energy tranfer donor. Various embodiments of the invention employ fluorescence resonance energy transfer (FRET). Other embodiments of the invention employ luminescence resonance energy transfer (LRET) or bioluminescence resonance energy transfer (BRET).

Fluorescence resonance energy transfer (FRET) is a distance-dependent interaction between the electronic excited states of two molecules in which excitation is transferred from a donor molecule to an acceptor molecule without emission of a photon, resulting in emission from the FRET acceptor. The efficiency of FRET is dependent on the inverse sixth power of the intermolecular separation, making it useful over distances comparable with the dimensions of biological macromolecules. In order for FRET to occur the donor and acceptor must be in very close proximity, e.g., less than approximately 10 nm. In addition, the absorption spectrum of the acceptor must overlap the fluorescence emission spectrum of the donor. The donor and acceptor dipoles should also be appropriately oriented. FRET measurements involve detecting acceptor emission, donor quenching (decreased emission from the FRET donor), and/or an alteration in the fluorescence lifetime of the donor. Nonfluorescent acceptors, also referred to as quenchers, include dabcyl and QSY dyes. Such molecules are capable of absorbing the energy of an excited fluorescent label when located in close proximity and of dissipating that energy without the emission of visible light. The invention, in various embodiments, makes use of increases in acceptor emission, decreases in acceptor emission, donor quenching, reduction in donor quenching, and/or increase or decrease in fluorescence lifetime of the donor to detect ion channel activity and/or to detect increased proximity or decreased proximity of two or more polypeptides from each other.

LRET is similar to FRET in many respects but uses a luminescent lanthanide as the energy-transfer donor. BRET is analogous to fluorescence resonance energy transfer (FRET), but uses a luminescent biomolecule such as luciferase, aequorin, or a derivative thereofas an energy donor and a fluorescent biomolecule such as GFP as the acceptor, thus eliminating the need for an excitation light source (68). BRET²™ is a form of BRET that is based on the transfer of resonant energy from a bioluminescent donor protein to a fluorescent acceptor protein using Renilla luciferase (Rluc) as the donor and a mutant of the Green Fluorescent Protein (GFP2) as the acceptor molecule. The BRET² signal is generated by the oxidation of DeepBlueC (Perkin-Elmer), a coelenterazine derivative that maximizes spectral resolution for superior sensitivity.

In certain embodiments of the invention a nanosensor component comprises a nanoparticle, a FRET donor moiety, and a recognition domain. The nanosensor component is introduced into a cell and attaches to a polypeptide of interest, e.g., an ion channel subunit. The cell is also loaded with an ion-sensitive fluorescent moiety that acts as a FRET acceptor. For example, the cell may be loaded with an ion-sensitive dye. Alternately, the cell may express an ion-sensitive fluorescent protein. The dye or protein emits a signal only if (i) the ion is present in sufficient concentration and; (ii) the dye or protein is excited by energy of an appropriate wavelength. The cell is exposed to electromagnetic radiation that excites the FRET donor but not the FRET acceptor. The FRET donor emits light at a suitable frequency to excite the FRET acceptor. If the ion channel is or has recently been active, the local ion concentration in the vicinity of the FRET donor attached to the ion channel is high. Therefore, FRET acceptors located in close proximity to the ion channel emit light at their preferred emission frequency while FRET acceptors located elsewhere in the cell do not produce a detectable signal.

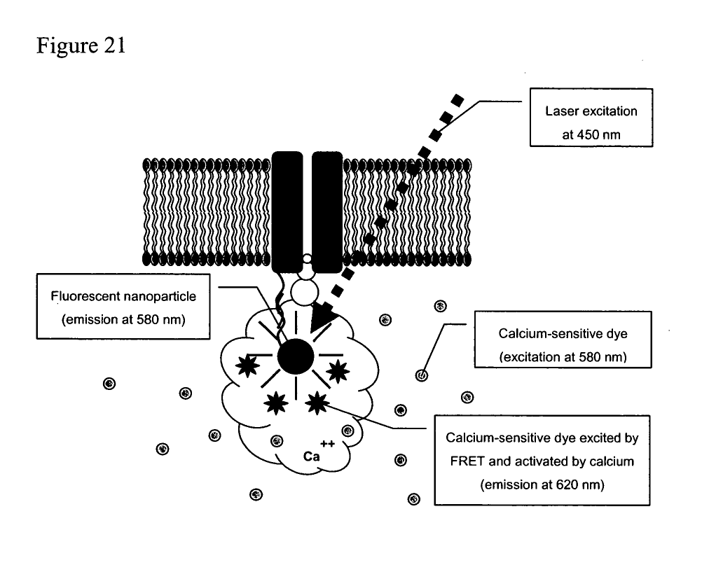

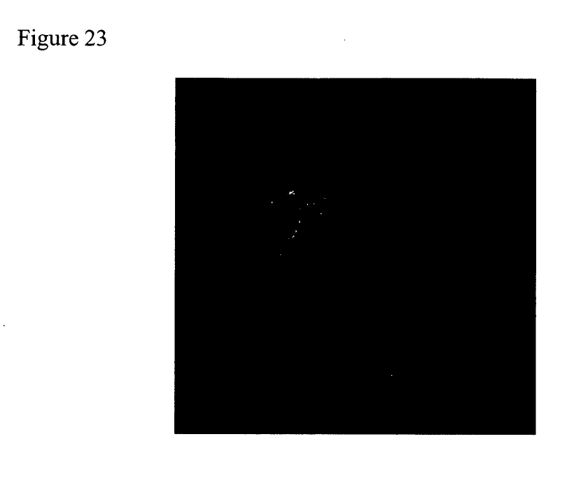

FIG. 21 shows a schematic representation of a nanoscale sensor for ion channel activity that utilizes FRET. Cells are also loaded with a calcium-sensitive dye whose absorption spectrum is chosen to coincide with the emission spectrum of the nanoparticle (580 nm). Typically the absorption peak and emission peak should coincide. The cell is then illuminated at the excitation frequency of the nanoparticle (450 nm laser). When calcium flows through the channel (channel opening), only the dye molecules in close proximity to the mouth of the channel emit light (at 620 nm), because they are: (a) in the presence of calcium and (b) in close proximity of the nanoparticle (allowing FRET). The optical signal at 620 nm can thus be reliably attributed to the presence of calcium at the mouth of the channel (implying channel activation), since the laser source cannot by itself excite the calcium-sensitive dye. Calcium-sensitive dye molecules that are far from the nanoparticle (even if they are in the presence of calcium, which may be coming from unrelated sources) do not emit any signal, as they are not excited at the proper frequency (580 nm) by FRET. Detecting calcium fluxes only in the immediate vicinity of the ion channel ensures that only specific ion channels of interest are monitored. For example, opening of a calcium channel to which no nanoparticle is attached would not result in emission of light from dye molecules in the vicinity of that channel because there would be no FRET donor in close proximity to the dye molecules. Similar designs utilize LRET or BRET donor and acceptor moieties. The dye can be a high affinity dye, a low affinity dye, a ratiometric dye, etc., in various embodiments of the invention. See Handbook of Molecular Probes, supra, for discussion and dyes having these properties. See also discussion in Takahashi (77).

In the embodiment shown in FIG. 21, both the FRET donor and acceptor are located inside the cell. In another embodiment of the invention, a nanoscale sensor component comprising a FRET donor binds to an extracellular domain of an ion channel subunit. The cell is loaded with an ion-sensitive dye and illuminated at the excitation frequency of the FRET donor. FRET takes place across the membrane, exciting the ion-sensitive dye, which emits a signal upon ion channel activity. The recognition domain can be an antibody that binds to an extracellular portion of the ion channel. Alternately, any of the recognition domain/target domain pairs described above can be employed.

In another embodiment of the invention a first nanosensor component comprising a nanoparticle and a FRET donor moiety and a second nanosensor component comprising a nanoparticle and a FRET acceptor moiety are used to detect ion channel activity. The first nanosensor component comprises a first recognition domain that binds to a first target domain in an ion channel subunit and the second nanosensor component comprises a second recognition domain that binds to a second target domain in an ion channel subunit. Alternately the first and second target domains can be located in different subunits of a single ion channel. The first and second target domains are typically different. The nanoparticles of the first and second nanoparticle sensor components comprise an ion-responsive binding moiety. The nanoparticle sensor components are introduced into cells and bind to the ion channel subunit(s). Preferably the nanoparticles are located too far from one another for FRET to occur in the absence of ion channel activity. An influx of an ion results in the nanoparticles coming into close proximity to one another, allowing FRET to occur when the cells are illuminated at the excitation frequency of the FRET donor. Detection of light emitted by the FRET acceptor serves as a signal that ion channel activity has occurred.

A large number of molecules and other moieties capable of acting as RET (FRET, BRET, or LRET) donors and acceptors are known in the art and are of use in various embodiments of the invention. FRET donors and acceptors include molecules in various classes such as acridine dyes, Alexa dyes, cyanine dyes, fluorescein and derivatives thereof, rhodamine derivatives thereof, etc. A nonlimiting list of exemplary FRET donor and acceptor pairs is presented below:

| Donor | Acceptor |
|---|---|
| Fluorescein | Tetramethylrhodamine; |
| | Texas Red |
| IAEDANS | Fluorescein |
| EDANS | Dabcyl |
| Fluorescein | Fluorescein |
| BODIPY FL | BODIPY FL |
| Fluorescein | QSY 7 and QSY 9 dyes; |
| Cy3 | Cy5 |
| FITC | Alexa fluors |
| Alexa fluors | GFP |
| BFP | GFP;YFP |
| CFP | YFP |
| GFP | Rhod-2 |

Numerous fluorescent or chemiluminescent dye molecules and proteins that are sensitive to particular ions are of use in the present invention. See, e.g., Handbook of Molecular Probes, supra. Many of the fluorescent, calcium-sensitive indicators are derivatives of calcium chelators such as EGTP, APTRA, and BAPTA. Exemplary calcium-sensitive dyes, among the dozens or more that are commercially available, are: fura dyes (e.g., fura-2, fura-4F, fura-5F, fura-6F, fura-FF, Fura Red), fluo dyes (e.g., fluo-3, flou-4), indo dyes (e.g., indo-1), rhodamine dyes (e.g., rhod-2, X-rhod-1), Oregon Green 488, Calcium Green, Calcium Crimson, quin-2, etc. A number of these dyes and proteins and their properties are reviewed by Takahashi (77). Fluorescent magnesium indicators include mag-fura-2, mag-fura-4, mag-fura-5, mag-fluo-4, mag-indo-1, mag-rhod-2, Magnesium Orange, and Magnesium Green. Fluorescent chloride indicators include Lucigenin and a variety of 6-methoxyquinolinium derivatives such as 6-Methoxy-N-(3-sulfopropyl)quinolinium (SPQ), N-(Ethoxycarbonylmethyl)-6-methoxyquinolinium bromide (MQAE) and 6-Methoxy-N-ethylquinolinium iodide (MEQ).

SBFI and PBFI are fluorescent indicators for sodium and potassium, respectively. Additional indicators for these ions include Sodium Green Na⁺ indicator, CoroNa Green Na⁺ Indicator, and CoroNa Red Na⁺ Indicator (all from Invitrogen).

In order to achieve ion sensing it is important to select an ion-sensitive FRET acceptor and a FRET donor that is capable of exciting the FRET acceptor. One of ordinary skill in the art will be able to select appropriate FRET donor and acceptor pairs. For example, suitable FRET donor and acceptor pairs for sensing calcium or sodium ions are listed below:

| FRET donor | FRET acceptor |
|---|---|
| FRET pairs for detection of | |
| calcium: | |
| Evident QD “Lake placid | Fluo-4 pentapotassium salt (absorption at |
| blue” (absorption | 493 nm, emission at 516 nm), or Fluo-5N |
| at 400 nm, emission at | (absorption at 493 nm, emission at 518 |
| 490 nm) (Evident | nm) (Molecular Probes/Invitrogen) |
| Corp.) | |
| Evident QD “Birch Yellow” | X-rhod-1, tripotassium salt (absorption at |
| (absorption at | 580 nm, emission at 602 nm) (Molecular |
| 400 nm, emission at 580 nm) | Probes/Invitrogen) |
| (Evident Corp.) | |
| Evident QD “Birch Yellow” | Calcium Crimson; absorption ˜ 580 nm, |
| (absorption at | emission ˜ 620 nm). |
| 400 nm, emission at 580 nm) | |
| (Evident Corp.) | |
| FRET pairs for detection of | |
| sodium: | |
| Evident QD “Lake placid | CoroNa Green (absorption at 492 nm, |
| blue” (absorption | emission at 516 nm) (Molecular |
| at 400 nm, emission at | Probes/Invitrogen) |
| 490 nm) (Evident | |
| Corp.) | |
| Evident QD “Catskill green” | CoroNa Green (absorption at 551 nm, |
| (absorption at | emission at 576 nm) (Molecular |
| 400 nm, emission at 540 nm) | Probes/Invitrogen |
| (Evident Corp.) | |

A variety of different nanoparticles are of use in these embodiments of the invention. For example, silica-containing particles or particles composed at least in part of organic polymers can be used. In one embodiment the particles comprise mesoporous silica. Certain particles have sufficient optical transparency to allow efficient excitation of and/or emission of light from moieties located in their interior. The FRET donor can be associated with such a nanoparticle in a variety of ways. For example, the FRET donor can embedded or entrapped within the particle or covalently or noncovalently bound to its surface, etc.

Note that quantum dots are themselves nanoparticles. Therefore in those embodiments of the invention that use quantum dots as a RET donor or acceptor the term “nanoparticle” can refer to a quantum dot or to a larger nanoparticle that comprises one or more quantum dots. For example, quantum dots can be encapsulated in or attached to a silica nanoparticle or can be provided as part of a polymeric nanocomposite.

In one embodiment, the FRET donor and acceptor are gold nanoclusters. Highly fluorescent gold quantum dots (nanodots) may be produced via reduction of Au⁺ ions in aqueous solution in the presence of dendrimers. Initially, in the absence of the reducing agent, gold ions migrate within the pores of such dendrimers and—upon reduction to Au—they form well-encapsulated Au nanoclusters, ranging in size from a few Au atoms to a few hundred (76). These QDs have a high quantum yield (˜40% for Au₈). In addition, these dendrimer-encapsulated gold nanoclusters exhibit narrow spectra in both excitation and emission, making them ideal FRET pairs. An exemplary FRET pair is Au₈ and Au₁₃. The former has an excitation frequency of ˜380 nm and emission at ˜450 nm (blue); the latter is excited at ˜440 nm (the frequency emitted by the first) and emits at ˜520 nm (green). FIG. 22 is a schematic representation of the operation of a nanosensor for detection of ion channel activity inside cells using Au nanocluster-based FRET. Two Au nanoclusters, both encapsulated in dendrimers coated with a moiety containing carboxyl groups, are attached to the target domains via corresponding recognition domains. One of the Au nanoclusters (e.g., a blue-emitting nanocluster) serves as a FRET donor while the other Au nanocluster (e.g., a green-emitting nanocluster) serves as a FRET acceptor. Upon channel opening, resulting in an increase in Ca⁺⁺ concentration, the two Au nanoclusters come into close proximity due to calcium chelation. This allows the FRET donor to excite the FRET acceptor. The system is illuminated at 380 nm (ultraviolet light) to excite the FRET donor, and the emitted light is filtered by a band-pass filter, e.g., at ˜520 nm (green). When the channel opens, the two Au nanoclusters come together so that the FRET acceptor (green-emitting) is excited by the FRET donor (blue-emitting), resulting in a flash of green light. Other FRET donor and acceptor pairs are also of use in this design. Similar designs utilize LRET or BRET donor and acceptor moieties.

In another embodiment, a nanoparticle comprising a signal-generating polypeptide that contains a a calcium-sensitive RET donor portion and a RET acceptor portion is attached to a calcium channel subunit. For example, a chimeric GFP-aequorin protein contains a calcium-sensitive RET donor (aequorin) and a RET acceptor (GFP) capable of being excited by aequorin (85). The signal-generating polypeptide is attached to the nanoparticle in any suitable manner, e.g., by coating the nanoparticle, covalent attachment, etc. Upon calcium channel activity, the RET donor emits at its emission frequency, exciting the RET acceptor. The ability of the nanoparticle to specifically direct a large number of signal-generating-polypeptides to the ion channel of interest results in an enhanced signal relative to systems in which individual polypeptides are used as calcium sensors. In a related embodiment, only one member of the RET pair (e.g., either aequorin or GFP) is provided by the nanoparticle while the other ion channel of interest is genetically engineered to contain the other member of the RET pair.

In another embodiment of the invention a first nanosensor component comprising a nanoparticle and a FRET donor moiety and a second nanosensor component comprising a FRET acceptor moiety are used to detect protein interactions. The first nanosensor component is attached to a first polypeptide of interest via a first recognition domain/target domain interaction. The second nanosensor component is attached to a second polypeptide of interest via a second recognition domain/target domain interaction. The cells are illuminated at the excitation frequency of the FRET donor. Physical interaction of the polypeptides allows FRET to take place between the donor and the acceptor, resulting in emission of light at the emission frequency of the FRET acceptor, thereby allowing detection of the physical interaction between the polypeptides. It will be appreciated that one could also measure donor quenching or altered donor fluorescence lifetime. Similar designs utilize LRET or BRET donor and acceptor moieties.

In other embodiments, only one nanosensor component is used, comprising either a RET donor or acceptor. The nanoparticle is attached to one of the polypeptides of interest via recognition domain/target domain binding. The other member of the RET pair is provided by genetically engineering the second polypeptide to include a biological RET donor or acceptor polypeptide, e.g., GFP, aequorin, or luciferase.

D. Sensors Utilizing Fluorescent or Luminescent Nanoparticles

In another embodiment of the invention ion channel activity is detected using a nanosensor comprising a nanoparticle that comprises an ion-sensitive signal generating moiety such as a fluorescent or chemiluminescent dye or protein that is sensitive to the ion(s) to be detected. The nanoscale sensor component further comprises a recognition domain. As in the designs above, the nanoscale sensor component is contacted with cells. The nanoscale sensor component becomes attached to an ion channel subunit of interest comprising a target domain that corresponds to the recognition domain. The cell is illuminated with light at the excitation frequency of the signal generating moiety. The signal generating moiety emits a fluorescent or chemiluminescent signal upon influx of the ion into the cell.

In certain embodiments of the invention a silica-containing nanoparticle is used. The invention provides a nanoparticle made at least in part of silica and comprising an ion-sensitive fluorescent or chemiluminescent moiety. The fluorescent or chemiluminescent moiety can be covalently or noncovalently attached to the nanoparticle. Optionally the moiety is attached to a coating layer. The silica can be mesoporous. Suitable methods for producing silica-containing nanoparticles comprising a calcium-sensitive fluorescent dye are described in Examples 11 and 12. Similar methods are used to produce nanoparticles comprising dyes that are sensitive to other ions.

E. Detecting Activity of Channels that Have an Outwardly Directed Ion Flux

In certain embodiments of the invention ion channel activity is detected in the cellular compartment into which ions flow, which is typically the cytosol. However, any of the above sensor designs may be used to detect activity of ion channels that have an outwardly directed ion flux, i.e., that permit passage of ions from the cell cytosol to the exterior of the cell, such as many potassium channels. The cells are contacted with nanoscale sensor components comprising a recognition domain, but there is no need for their internalization. In certain embodiments of the invention an antibody that binds to an extracellular portion of the ion channel subunit is used as a recognition domain in which case the ion channel subunit can be a native ion channel subunit. In certain embodiments an ion channel subunit having a target domain located in an extracellular portion of the channel (following assembly of the channel) is engineered.

VI. Delivery of Nanosensor Components to Cells

In various embodiments of the invention detection of ion channel activity or protein interaction involves the delivery of nanosensor components to the cell interior, e.g., into the cytosol. Cells of various types are known to take up certain foreign particles, including nanoparticles, spontaneously, e.g., via endocytosis or other mechanisms. However, the efficiency may be relatively low. In addition, particles may be trapped within certain compartments, e.g., endosomes or lysosomes, which are topologically part of the cell exterior, rather than being delivered to the cytosol or to the membrane or interior of organelles such as the nucleus, mitochondria, etc. The invention features compositions and methods that provide for improved intracellular delivery of nanoparticles and nanosensor components. For purposes of the present invention, “intracellular delivery” refers to delivery of an entity such as a particle to either (i) the cell cytosol, or (ii) the interior of a compartment within the cell other than one in which it was internalized or (iii) a membrane enclosing a compartment other than one in which the entity was internalized. In certain embodiments of the invention the compartment to which the nanoparticle is delivered is not an endosome, pinosome, or lysosome.

In certain embodiments of the invention an electric field is applied to enhance intracellular delivery of a nanoparticle sensor component. Application of an electric field to cells to enhance their uptake of DNA, a technique referred to as electroporation, has long been known in the art (13, 14). The mechanism may involve temporary disruption of the cell membrane, allowing foreign bodies to enter, followed by resealing of the membrane. In the present invention electroporation is used to enhance the uptake of nanoscale sensor components by cells. Standard electroporation protocols such as those described in references 13 or 14 can be used. In certain embodiments, the protocol used to enhance intracellular delivery of a nanosensor component to a cell type of interest is one that, if used to enhance transformation of cells of that type by a typical high copy plasmid (e.g., a pUC-based plasmid), would enhance the number of transformants by at least 10-fold, at least 50-fold, or at least 100-fold, or at least 1000-fold relative to the number of transformants that would be obtained simply by incubating the cells with the plasmid for a period of time.

Parameters such as electric field strength, voltage, capacitance, and duration of pulse can be optimized for the delivery of nanoparticle sensor components of any particular size, shape, and composition, and the invention is in no way limited to parameters that have been successfully used to enhance cell transformation

In certain embodiments of the invention endosomal disruptors are used to enhance intracellular delivery of nanoparticle sensor components. Endocytosis is a process by which cells absorb material from their surrounding environment. Endocytosis consists of engulfment of substances in a pocket formed by the cell membrane that later migrates into the cell interior. Nanoparticles, once endocytosed, may remain trapped in such endosomes and would not be able to reach their target polypeptide. Release of particles from endosomes can be achieved by treatment of cells with endosomal disruptors, such as chloroquine (4) and NEM (N-ethylmaleimide) (5), and the like. These compounds disrupt the cell's endocytotic pathway and allow discharge of endosomal contents into the cytoplasm.

The invention provides nanosensor components that comprise a delivery moiety, by which is meant any moiety whose attachment (either covalent or noncovalent) to a second moiety such as a nanoparticle enhances intracellular delivery of the second moiety. A delivery moiety may enhance one or more aspects of delivery such as cellular entry (uptake), escape from an endosome, lysosome, or pinosome, or entry of the second moiety into a desired cellular compartment. Preferred delivery-enhancing moieties increase cellular entry (uptake), escape from an endosome or pinosome, or both.

In certain embodiments of the invention the delivery moiety comprises any of a variety of protein domains that are capable of inducing translocation of the whole parent protein inside cells. These domains are commonly referred to as protein transduction domains (PTDs) or cell penetrating peptides (CPPs). Cross-linking or otherwise attaching such peptides to various cargoes such as large proteins, plasmids, and nanoparticles significantly enhances their import into living cells (8, 12). For example, peptides derived from various viruses, DNA binding segments of leucine zipper proteins, etc., have been shown to have delivery-enhancing activity. Any of these domains are of use as a delivery moiety in the present invention. A diverse set of peptide sequences possessing delivery-enhancing properties are known. Many of these peptides are arginine-rich, e.g., they may consist essentially of between 5 and 50 amino acids, of which at least 50% are arginine. See, e.g., Langel, U. (ed.), “Cell-Penetrating Peptides: Processes and Applications”, CRC Press, Boca Raton, Fla., 2002, for further discussion.

The TAT₄₉₋₅₇ peptide, referred to herein as “TAT” or “TAT peptide” (sequence: RKKRRQRRR (SEQ ID NO: 18)) from the HIV-1 protein is a representative example (11). Other peptides of use are listed below:

YGRKKRRQRRRP (SEQ ID NO: 19) (a longer peptide containing TAT₄₉₋₅₇)

GRKKRRQRRRPPQ (SEQ ID NO: 20) (a longer peptide containing TAT₄₉₋₅₇)

RQIKIWFZQRRMKWKK (SEQ ID NO: 21) (from the Antennapedia protein)

GWYLNSAGYLLGK(e-Cys)INLKALAALAKKIL (SEQ ID NO: 22) (Transportan-27)

GWYLNSAGYLLGK(e-Cys)INLKALAAL (SEQ ID NO: 23) (Transportan-22)

GLFEALEELWEAK (SEQ ID NO: 24)

Peptides useful as delivery moieties may contain L-amino acids, D-amino acids, or both and may contain a variety of amino acid modifications.

Other deliverymoieties include peptide-like molecules known as peptoid molecular transporters (11 and U.S. Pat. Nos. 6,306,933 and 6,759,387). Certain of these molecules contain contiguous, highly basic subunits, particularly subunits containing guanidyl or amidinyl moieties.

Delivery moieties such as peptides can be attached to nanoparticles using any of the methods described above. Peptides can also stably adsorb to metal nanoparticle surfaces, e.g., via electrostatic interactions.

In certain embodiments of the invention the delivery moiety can be removed from the nanoparticle following intracellular delivery. A variety of means may be used to remove the probe. For example, removal can occur as a result of light-directed cleavage, chemical cleavage, protease-mediated cleavage, enzyme-mediated cleavage, etc. The delivery moiety may be attached to a nanoparticle via a cleavable linker, e.g., a linker that is readily cleavable under conditions that exist within cells. A number of linkers that result in production of readily cleavable bonds are known. The linker may contain a site for cleavage by a specific protease.

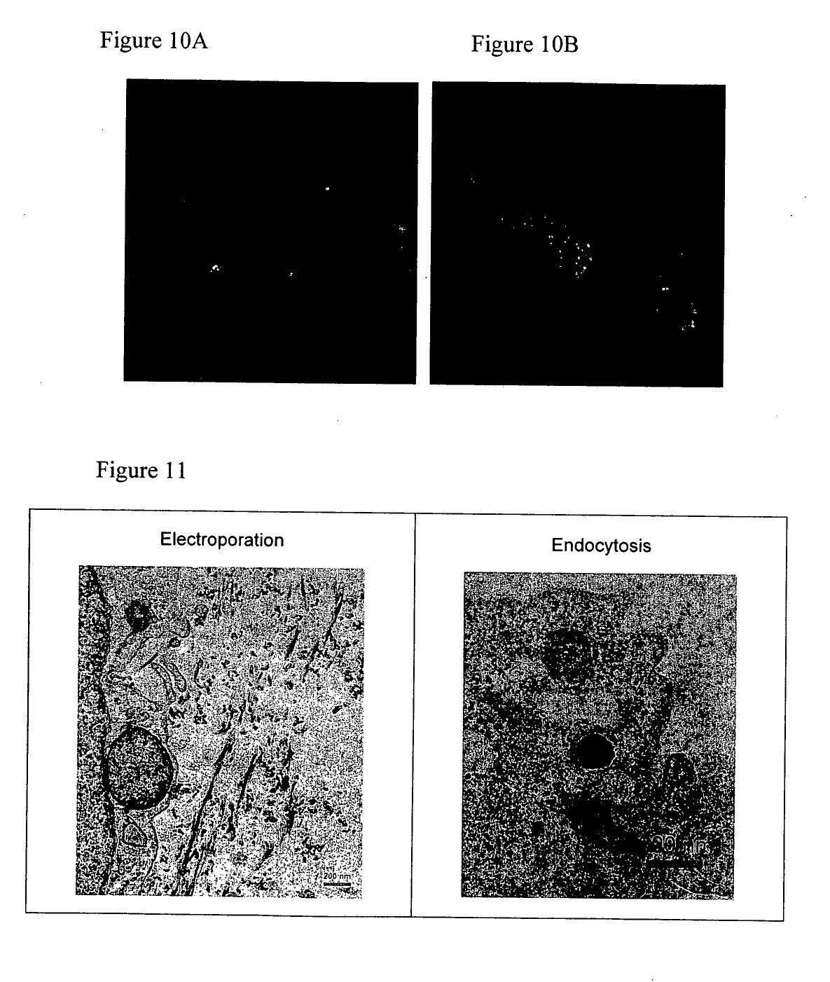

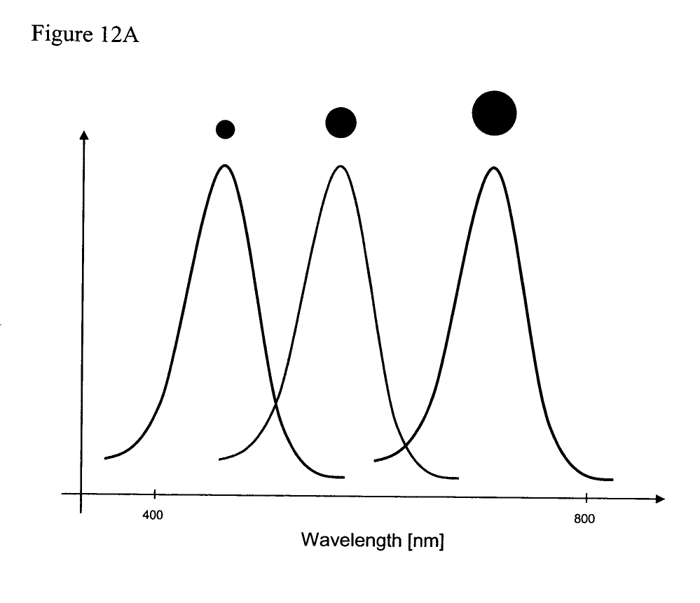

In certain embodiments of the invention electroporation is used to enhance intracellular delivery of a nanoparticle sensor component that comprises a delivery moiety. The exact translocation mechanism mediated by TAT has been elucidated only recently. It is now believed that TAT-fusion proteins are internalized by lipid-raft dependent macropinocytosis (9). The first phase of macropinocytosis involves an ionic cell-surface interaction: the positively charged TAT peptide engages the negatively charged cell membrane. After this event, the cell extends protrusions around the cargo and engulfs it in a vesicle, which is then transported inside. Without wishing to be bound by any theory, we reasoned that subjecting cells to an electric field at the early stages of macropinocytosis would make the cargo translocate across the membrane directly into the cytosol, without being trapped into vesicles. Our data indeed suggest that a combination of TAT-conjugation and application of an electric field surprisingly allows ready translocation of the cargo inside the cytosol, without intermediate endocytosis (FIGS. 10A, 10B, and 11). Regardless of the exact mechanism, our data suggest that application of an electric field in conjunction with an attached delivery moiety, significantly increases delivery of nanoparticles to the cell interior. While both electroporation and the use of TAT and other peptides having similar functions have previously been used independently to enhance nanoparticle uptake, to the best of the inventors' knowledge, the unexpected improvement in intracellular nanoparticle delivery resulting from the combination of these two approaches has not been previously reported. The invention therefore provides a method of introducing a nanoparticle into a cell comprising steps of: providing a nanoparticle having a delivery moiety attached thereto; providing a cell; and applying an electric field to the cell in the presence of the nanoparticle. Standard electroporation protocols can be used. Parameters such as electric field strength and duration of pulse can be optimized for the delivery of nanoparticle sensor components of any particular size, shape, and composition.

In other embodiments of the invention, magnetic forces are used to enhance cellular delivery of nanoparticles in addition to, or instead of, attachment of a delivery moiety and optional application of an electric field. In these embodiments the nanoparticle is made, at least in part, of a magnetic material. The particles are conjugated with appropriate binding moieties as described above. Suitable devices for applying such magnetic forces to cells in the presence of nanoparticles, and suitable magnetic field strengths, may be selected in accordance with the literature (15) and U.S. Ser. No. 09/895,019 (U.S. Publication No. 20020086842). In certain embodiments of the invention the nanoparticle comprises a delivery moiety. The invention therefore provides a method of introducing a nanoparticle into a cell comprising steps of: providing a nanoparticle having a delivery moiety attached thereto; providing a cell; and applying a magnetic field to the cell in the presence of the nanoparticle.

In any of the above methods, cells may be incubated with the nanosensor components for a period of time before application of the electric or magnetic field.

Other methods of introducing nanosensor components into cells may also be used including microinjection and particle bombardment (biolistics), in which particles are accelerated towards cells by a pressure pulse. Lipid-mediated delivery, e.g., using any of a variety of lipid agents typically used for transfecting cells can also be employed. Exemplary agents include commercially available transfection reagents such as Superfect™, Polyfect™, Effectene™, Lipofectamine, Oligofectamine, etc., and other lipids and mixtures thereof. In another embodiment, nanoscale sensor components are delivered using liposomes.

VII. Screening Methods and Reagents

The invention provides compositions and methods for screening to identify compounds, e.g., small molecules, peptides, peptide-like natural products, and proteins that modulate the activity of ion channels, GPCRs, G proteins, or any polypeptides that interact with one another. Ion channel modulators include, e.g., compounds that activate or inhibit ion channels and/or that alter ion channel selectivity. GPCR modulators include, e.g., compounds that activate or inhibit GPCRs or that alter their interaction with G proteins.

The methods can be used to identify compounds that modulate the activity of one or more ion channel species. By “ion channel species” is meant an ion channel that is composed of a specific set of subunits. The technology provides screening methods with a high signal-to-noise ratio and a high degree of specificity.

The invention allows detection of activity of a predetermined ion channel species that contains an ion channel subunit comprising a target domain as described above. The specificity of binding between a nanosensor component comprising a recognition domain and an ion channel subunit comprising a complementary target domain allows detection of the activity of ion channels of a specific, predetermined category, i.e., those containing a target domain that binds to the recognition domain of a nanoscale sensor component introduced into the cell. For example, an ion channel subunit that occurs naturally in a specific naturally occurring ion channel species may be engineered to include a target domain. The invention detects activity of a category of ion channel(s) consisting of ion channel species that comprise this subunit. The ion channel category comprising the predetermined ion channel subunit will often correspond exactly to a single naturally occurring ion channel species. However, this need not be the case. For example, if a particular subunit occurs naturally in two or more naturally occurring ion channel species, then the invention may detect activity of a category of ion channels consisting of one or more of these species, e.g., all of these species.

If desired, a nanosensor that binds to a target domain present in an ion channel subunit that is known in the art to be a component only of a particular ion channel species of interest can be used to detect only the activity of that particular species.

Certain embodiments of the invention employ nanoscale sensors in which two or more sensor components are attached either to a single ion channel subunit or to different ion channel subunits. The different subunits may either have the same original, unmodified sequence (e.g., prior to addition of the target domain) or may have different original sequences. It may be desirable to attach each of two or more components to different subunits of a single channel, thereby increasing the specificity of the sensor. For example, one nanoscale sensor component may be attached to an a subunit of a calcium channel and a second nanoscale sensor component may be attached to a γ subunit of a calcium channel. In these embodiments the two ion channel subunits having different sequences may include, or may be modified to include, different target domains. The nanoparticles targeted to each of these domains may have different optical properties, so that it is possible to distinguish the optical properties of sensors attached to different ion channel species and thus more specifically detect activity of these species.

When reference is made to the action of certain compounds to activate or inhibit a channel, such activation or inhibition may be direct (i.e., involves binding of the compound to the channel) or indirect (does not require binding of the compound to the channel). In general, a compound is considered to activate a channel if the effect of exposure of cells to the compound under any given set of conditions (e.g., in vitro or in vivo) is to increase the activity of the channel, by any means. A compound is considered to inhibit a channel if the effect of exposure of cells to the compound under any given set of conditions (e.g., in vitro or in vivo) is to decrease the activity of the channel, by any means. It is also noted that a variety of synonyms may be used to describe the effect of a compound on a channel. For example, a compound or treatment may be said to “stimulate”, “activate”, or “enhance” the channel or its activity if the effect of the compound or treatment is to increase the activity of the channel. Such compounds may be referred to as activators, stimulators, enhancers, etc. Similarly, a compound or treatment may be said to “inhibit”, “repress”, “block” the activity of the receptor or channel if the effect of the compound or treatment is to decrease the activity of the channel. Similar considerations apply to receptors.

Activators of ion channels or receptors include compounds that act as agonists or antagonists. The term “agonist” is intended to be used as is accepted in the art. In general, the term refers to a substance that can interact with (e.g. bind to) a channel or receptor and initiate a physiological or a pharmacological response characteristic of that induced by a physiological stimulus such as interaction of an endogenous ligand with the channel or receptor or any other physiologically relevant stimulus. The term “agonist” also refers to partial agonists, i.e., compounds that are capable of partially activating a channel or receptor, e.g., activating it to a lesser extent than an endogenous ligand or physiological stimulus. Inhibitors of ion channels or receptors include compounds that act as ion channel antagonists. The term “antagonist” is intended to be used as is accepted in the art. In general, the term refers to a substance that opposes the channel- or receptor-associated responses normally induced by a stimulus; e.g., those normally induced by another bioactive agent such as an endogenous ligand, or by a physiological stimulus. An antagonist may bind to a channel or receptor and prevent binding of an endogenous ligand that would normally activate the channel or receptor, or may prevent binding of another agonist to the channel or receptor. An antagonist may bind to a channel or receptor and alter its properties such that a stimulus that would normally activate the channel or receptor is no longer able to do so (e.g., may raise the threshold needed for activation). The antagonist may or may not induce an effect itself. It is noted that a number of ligand-gated ion channels are considered receptors and that the invention is also applicable to a variety of non-channel receptors such as GPCRs, e.g., when such receptors are involved in protein-protein interactions such as those between GPCR and G protein subunits. The use of the phrase “channel or receptor” is not intended to be limiting and is not intended to imply that these classifications are mutually exclusive.

The invention includes a method of testing a compound comprising steps of: (i) providing a cell comprising an ion channel having a nanosensor component attached thereto; (ii contacting the cell with the compound; (iii) gathering information indicative of ion channel activity or lack thereof from the nanoscale sensor; and (iv) determining whether the compound is a modulator of ion channel activity based on the information gathered in step (iii). The nanosensor component either alone or in combination with one or more additional components (e.g., nanosensor components or signal-generating moieties) forms an ion-sensitive nanosensor.

The invention further provides a method of detecting ion channel or GPCR activity or lack thereof comprising steps of: (i) providing a cell comprising a polypeptide having a nanosensor component attached thereto, wherein the polypeptide is an ion channel subunit or GPCR, and wherein the nanosensor component comprises a nanoparticle; (ii) maintaining the cell under conditions in which ion channel or GPCR activity may occur; and (iii) detecting a signal that is indicative of ion channel or GPCR activity or lack thereof. The method may generally be applied to detect interaction between any two polypeptides.

“Contacting a cell” with a compound means that the compound is provided to the cell in any suitable manner. Typically the compound is added to culture medium in which the cell is cultured or is administered to an organism in which the cell is located. Maintaining a cell under conditions under which ion channel activity, receptor activity, or protein protein interaction can occur is an optional step in any of the methods of the invention and means that the cell is maintained for any period of time under conditions consistent with preserving the integrity of the cell membrane, and preferably, maintaining cell viability, for the length of time required to perform the method. The conditions can be, e.g., standard culture conditions for the cell and can include the presence of one or more test compounds, the presence of one or more known modulators of ion channel activity, receptor activity, and/or protein protein interaction, the manipulation of ion concentration(s) in the extracellular environment, etc. Gathering information can include any aspect of detecting and/or measuring activity of an ion channel or GPCR. The information may be qualitative or quantitative. The step of gathering information may comprise determining whether an ion channel opening or closing event has occurred and/or may comprise determining any one or more of the parameters of ion channel activity mentioned above. Information may be gathered by microscopic means, by obtaining an extinction, scattering, and/or absorption spectrum, by detecting fluorescence or chemiluminescence, by measuring a relaxation time or magnetic field, etc.

The invention also provides a method of identifying or detecting a modulator of ion channel activity comprising steps of: (i) providing an ion channel having a nanosensor component attached thereto, wherein the nanosensor component either alone or in combination with one or more additional components forms an ion-sensitive nanosensor; (ii) contacting the ion channel with a compound; (iii) gathering information indicative of ion channel activity or lack thereof from the nanoscale sensor; and (iv) determining whether the compound is a modulator of ion channel activity, or detecting a modulator of ion channel activity, based on the information gathered in step (iii). In preferred embodiments of the invention the ion channel is present within a cell (e.g., the ion channel spans a cell membrane of a cell).

The above method may be used to identify an ion channel modulator, e.g., to test a specific compound to determine whether it is an ion channel modulator or to measure its effect on ion channel activity, to test a collection of compounds to determine which of them modulate ion channel activity, etc. Alternately, the method can be used to determine whether an ion channel modulator is present in the environment or in a sample to which the cell is exposed. Many toxins (e.g., neurotoxins), including a variety of biowarfare agents, act by modulating ion channel activity. The invention may be used to detect the presence of such agents. A device comprising a nanoscale sensor of the invention, one or more cells, and a suitable system for detecting a signal from the cell(s) can be deployed in an area where a toxin may be present, e.g., a monitoring station, a combat zone, an industrial site, etc. The invention provides miniaturized devices comprising a nanoscale sensor, one or more cells, and a readout mechanism. Such devices could be easily carried into the battlefield or positioned at a remote location. Wireless means of transmitting a signal indicative of the presence of a toxin can be included. In other embodiments ion channels are assembled within artificial membranes (e.g., lipid bilayer membranes, which may be composed of naturally occurring or synthetic lipids) that are capable of supporting assembly and operation of the ion channel. The membranes, appropriately housed in a protective housing, are positioned at a location in which it is desired to monitor for presence of a toxin. Upon exposure to a toxin that activates or inhibits the particular ion channel type to which the sensor is attached, a change in optical and/or magnetic properties results, allowing the presence and/or amount of a toxin that has such an effect to be detected.

The methods may comprise a step of comparing ion channel activity in a cell that is contacted with the compound (a test cell) with ion channel activity in control cell. The control cell can be a cell that is not contacted with the compound or is contacted with a lesser amount of the compound. The method may include a step of gathering information indicative of ion channel activity or lack thereof from a control cell. The control cell is generally similar to the test cell, e.g., it is generally of the same cell type and typically expresses the same ion channel type as that whose activity is measured in the test cell. Generally the control cell also expresses the recombinant ion channel subunit expressed by the test cell. The control cell optionally contains a nanoscale ion channel sensor, which may be used to gather information regarding ion channel activity in the test cell. However, other methods of gathering such information can also be used. A historical control can be used, i.e., it is not necessary to actually measure ion channel activity in a control cell.

The method may comprise a step of comparing ion channel or receptor activity in cells that are contacted with the compound with ion channel or receptor activity in cells that are contacted with a different compound, e.g., a known ion channel activator or inhibitor. The compound is identified as an activator if contacting the ion channel or receptor with the compound causes an increase in activity of the channel or receptor relative to that which occurs in a suitable control cell not contacted with the compound. The compound is identified as an inhibitor if contacting the ion channel or receptor with the compound causes a decrease in activity of the channel or receptor relative to that which occurs in a suitable control cell not contacted with the compound. Activators and inhibitors of a large number of ion channels and receptors, including agents that selectively or specifically activate or inhibit particular channels or receptors are known in the art. See, e.g., Alexander, S., supra.

In certain embodiments of the invention an approved therapeutic agent (e.g., an agent approved by the FDA or an equivalent regulatory agency) known or believed to act on an ion channel or receptor is tested using a method of the invention to determine its activity on one or more ion channel or receptor species. In certain embodiments of the invention the effect of a test compound on ion channel or receptor activity is compared with the effect of an approved therapeutic agent using a method of the invention. See, e.g., Goodman & Gilman, supra, or Katzung, supra, for numerous therapeutic agents that act at least in part by modulating one or more ion channels or receptors.

The above methods may also be applied to detecting and/or identifying modulators of receptors such as GPCRs.

One of ordinary skill in the art will be able to select appropriate conditions for performing the methods of the invention. For example, it may be useful to maintain cells under conditions in which an ion of interest is depleted and then to contact cells with the ion while monitoring ion channel activity. A test cell may be treated with a physiological or pharmacological stimulus or inhibitor prior to contacting the cell with a test compound. For example, a cell may be contacted with an agent that increases or decreases permeability of the cell to an ion of interest by a mechanism that does not involve activation or inhibition of an ion channel whose activity is to be tested. An ionophore, e.g., a calcium ionophore may be used.

In certain embodiments of the invention the test cell is contacted with a known activator or inhibitor of the ion channel whose activity is to be detected and also with a test compound. Contacting the test cell with a known activator will increase ion channel activity and may make it easier to identify compounds that inhibit the ion channel (e.g., if there is a low level of spontaneous activity). Conversely, contacting the test cell with a known inhibitor will decrease ion channel activity and may make it easier to identify compounds that activate the channel (e.g., if there is a high background level of activity). The ability of the test compound to increase or decrease the effect of the known activator or inhibitor is assessed. For example, a cell may be contacted with a known calcium channel activator and with a test compound. The compounds may be applied in either order and may be present at the same time (concurrently), or one compound may be removed (e.g., by washing the cells), prior to addition of the second compound. If the test compound decreases calcium channel activity in cells that have been contacted with the activator, relative to the calcium channel activity that is observed in cells contacted with the activator but without the test compound, the test compound may be identified as a calcium channel inhibitor.

In certain embodiments of the invention a cell is treated with a test substance at a first concentration and ion channel activity is measured. The cell (or a different, comparable cell) is also treated with the same substance at a higher or lower concentration, and ion channel activity is measured. If ion channel activity is higher following treatment at the higher concentration than following treatment at the lower concentration, then the substance is identified as an ion channel activator. Conversely, if ion channel activity is lower following treatment at the higher concentration than following treatment at the lower concentration, then the substance is identified as an ion channel inhibitor.

Similar considerations and methods may be applied to receptors such as GPCRs.

In any of the foregoing methods, compounds can be used at a range of concentrations. Typical concentrations range from nanomolar to millimolar. Ion channel or receptor activity may be monitored in the presence of a test compound or after its removal. In certain embodiments test compounds are added to wells in a caged form so that they can be rapidly and synchronously released after becoming distributed throughout the fluid in the well. In certain embodiments the wells initially contain a low concentration of an ion of interest. The ion of interest is added in a caged form so that it can be rapidly and synchronously released after becoming distributed throughout the fluid in the well. Release of the compound or ion is achieved, e.g., using flash photolysis, UV irradiation, X-ray-induced radiolysis, etc.

Although the above methods have generally been described in terms of single cells, it will be appreciated that they will typically be performed using populations of cells, and results may be expressed in terms of averages. In certain embodiments of the invention the methods are performed in a high-throughput format using techniques that are well known in the art, e.g., in multiwell plates, using robotics for sample preparation and dispensing, etc. According to these approaches cells, or populations of cells, are dispensed into individual vessels, e.g., wells in a multiwell plate. One or more test compounds is added to each well, and information about ion channel or receptor activity is gathered. Representative examples of various screening methods may be found, for example, in U.S. Pat. No. 5,985,829, U.S. Pat. No. 5,726,025, U.S. Pat. No. 5,972,621, and U.S. Pat. No. 6,015,692. The skilled practitioner will readily be able to modify and adapt these methods as appropriate. Methods for gathering spectra, images, and other signals from multiwell plates are well known in the art.

Compounds suitable for screening according to the above methods include small molecules, natural products, peptides, nucleic acids, etc. Sources for compounds include natural product extracts, collections of synthetic compounds, and compound libraries generated by combinatorial chemistry. Libraries of compounds are well known in the art. One representative example is known as DIVERSet™, available from ChemBridge Corporation, 16981 Via Tazon, Suite G, San Diego, Calif. 92127. DIVERSet™ contains between 10,000 and 50,000 drug-like, hand-synthesized small molecules. The compounds are pre-selected to form a “universal” library that covers the maximum pharmacophore diversity with the minimum number of compounds and is suitable for either high throughput or lower throughput screening. For descriptions of additional libraries, see, for example, Tan, et al., “Stereoselective Synthesis of Over Two Million Compounds Having Structural Features Both Reminiscent of Natural Products and Compatible with Miniaturized Cell Based Assays”, Am. Chem Soc. 120, 8565-8566, 1998; Floyd C D, Leblanc C, Whittaker M, Prog Med Chem 36:91-168, 1999. Numerous libraries are commercially available, e.g., from AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc.

Molecular modeling can be used to identify a pharmacophore for a particular target, i.e. the minimum functionality that a molecule must have to possess activity at that target. Such modeling can be based, for example, on a predicted or known structure for the target (e.g., a two-dimensional or three-dimensional structure). Software programs for identifying such potential lead compounds are known in the art, and once a compound exhibiting activity is identified, standard methods may be employed to refine the structure and thereby identify more effective compounds. Structures of a number of ion channels and receptors are known in the art. In addition, known ion channel or receptor modulators can be modified, and the effects of the modified compounds can be assessed. The invention therefore includes methods leading all the way from identification of a lead compound intended to be a therapeutic agents that modulates ion channel or receptor activity, through optimization and selection and testing of a development candidate, and beyond.

Compounds identified as modulators of ion channel or receptor activity may fall into any therapeutic category. Categories of agents discoverable using the methods of the invention include, but are not limited to, antihypertensive agents, antiarrhythmic agents, anesthetics, antispasmodic agents, muscle relaxants, analgesic agents, anticonvulsants, antidiabetic agents, hypnotic or anxiolytic agents, diuretic agents, antipsychotic agents, antidepressive agents, antiinflammatory agents, immunosuppressive agents, agents that inhibit ischemia-reperfusion injury, agents that inhibit or enhance apoptosis, etc.

In addition to methods and reagents for identifying and testing compounds, the invention provides tools for investigating ion channel structure and its effects on ion channel activity and selectivity. For example, the recombinant ion channel subunit containing a target domain may contain one or more additional alterations with respect to the naturally occurring subunit. The alteration can be an addition, deletion, or substitution of one or more amino acids. Methods for producing polynucleotides that encode such altered ion channel subunits are known in the art. The activity of an ion channel containing the altered recombinant ion channel subunit can be detected and compared with the activity of a channel in which the recombinant ion channel subunit does not have the alteration, thereby identifying the effect of the alteration on ion channel activity. Alternately, or in addition to, engineering an alteration in the recombinant ion channel subunit that contains the target domain, one or more of the other subunits known to be present in an assembled ion channel together with the naturally occurring form of the recombinant ion channel subunit containing a target domain can be altered by addition, deletion, or substitution of one or more amino acids. The activity of an ion channel containing both a recombinant ion channel subunit and a second altered ion channel subunit can be detected and compared with that of a channel in which the second ion channel subunit is not altered (or has a different alteration). The effect of a compound on an ion channel containing the second altered subunit may be compared with the effect of the compound on an ion channel that is not altered. This approach may identify amino acid(s) and/or domains of the second ion channel subunit that are particularly important for drug action and may help identify target sites within the channel for molecular drug design and/or suggest modifications of existing agents to alter (e.g., increase) their effect on ion channel activity.

The above methods may be applied to ion channels of any kind. Numerous ion channel subunits have been cloned, and the subunit composition of a large number of channels is known, as discussed above. Similar methods may be applied to investigate GPCR and/or G protein structure, function, and/or interactions. The methods are not limited to ion channels, transporters, or receptors that have been identified as such in the art but are also applicable to ion channels, transporters, and receptors that have yet to be discovered. In addition, although it is envisioned that one of the advantages of the invention lies in its ability to allow detection and monitoring of ion channels of a specific ion channel species, the methods can also be used in a less specific manner. For example, although ion channel activity has its greatest effect on ion concentration near the mouth of the channel, it also causes an overall increase in ion concentration within the cell, particularly if sustained over a period of time. Such an increase may be sufficient to cause changes in optical and magnetic properties of nanoparticles (e.g., by inducing aggregation of nanoparticles) even when none of the nanoparticles is attached to the ion channel. Such changes may be detected as described above. In addition, the nanosensors can be used to detect and/or monitor ions in any environmental setting or sample.

A variety of signal and/or data processing tools (e.g., software, hardware, and combinations thereof) may be used to facilitate any of the steps in the above-described methods including detection of optical or magnetic signals, analysis and presentation of data obtained thereby, etc. Such tools and their use together with the above methods comprise an aspect of the invention. Signal and/or data processing algorithms may be applied to a signal detected from a single cell or population of cells. As one example, algorithms useful for distinguishing scattering by a plasmon resonant particle from background scattering or scattering from other entities are known. Algorithms are typically encoded in software, which may be written in a variety of programming languages and may be provided in computer-readable format on a computer-readable medium. A computer-readable medium containing, e.g., storing, such algorithms, and a computer programmed to execute them, are additional aspects of the invention Alternately, some algorithms may be embodied in hardware. Additional hardware components may also be employed. For example, signals may be filtered or otherwise processed in hardware or software. In addition, the gathering of spectra and/or images for purposes of high throughput screening is typically computer-controlled. In general, methods for doing so analogous to those currently used for high throughput screening can be used (36).

The invention further provides a computer-readable medium containing, e.g., storing, information obtained from performing any of the screening methods of the invention, regardless of where such screening methods were practiced, and a computer capable of processing, displaying, or manipulating the information. The information can be expressed in any convenient format. Typically the information will be stored in a database. The information may, for example, identify compounds by name, structure, identication number, or any other suitable means. The information may identify ion channels (e.g., ion channels species, ion channel subunits, etc.)., that are activated or inhibited by the compounds. The ion channels or subunits may be identified in any suitable manner. The information may identify one or more compounds that activate or inhibit a particular ion channel or receptor. The information can be qualitative or quantitative.

The invention further provides a computer or other electronic device capable of processing, transmitting, or receiving the information and also includes a method comprising the step of transmitting, receiving, or processing the information, preferably by electronic means or using the information in a concrete and substantial manner, regardless of where the information was originally gathered. The invention further provides a method comprising the step of testing a compound identified according to any of the methods described herein either in a cell-based assay, in an animal model (e.g., an animal model of disease), or in a human subject, or of synthesizing or modifying a compound identified according to any of the methods described herein.

The invention provides systems for performing high throughput screening that include apparatus for performing the methods, e.g., robots, plate readers, software, and/or computers as described above. The systems are typically provided for use with nanosensor components and may be specially adapted for use therewith.

The invention further provides a variety of kits. The kits may, for example, allow the user to generate a recombinant polypeptide of interest comprising a recognition or target domain and/or to detect activity of a polypeptide (e.g., a recombinant polypeptide) using any of the methods described above. Certain of the kits include at least one nanosensor component comprising a nanoparticle having a recognition domain attached thereto. The nanoparticle may have a ligand-responsive moiety and/or a delivery-enhancing moiety attached thereto. The recognition domain is able to specifically bind to a heterologous target domain of a recombinant cellular polypeptide, e.g., a recombinant ion channel. A nucleic acid encoding a recombinant polypeptide, preferably comprising a target domain that specifically binds to a recognition domain attached to the nanoparticle, may be included in the kit. A cell comprising a recombinant polypeptide, preferably comprising a target domain that specifically binds to a recognition domain attached to the nanoparticle, may be included in the kit. Other components include, but are not limited to, components selected from the group consisting of: an ion channel activator, an ion channel inhibitor, a receptor activator, a receptor inhibitor, an ionophore, a fluorescent or luminescent moiety, an ion-sensitive dye or protein, a culture medium, a nucleic acid sequence that encodes a target domain, a nucleic acid that encodes a recognition domain, a vector comprising a nucleic acid sequence that encodes a target domain, a vector comprising a nucleic acid sequence that encodes a recognition domain, and instructions for use. The kits may contain multiple different nanoscale sensor components, e.g., components comprising different recognition domains and may contain corresponding recombinant polypeptides, etc. The nanoscale sensor components can be provided in any suitable form. For example, they may be provided in lyophilized form.

Kits may include one or more vessels or containers so that certain of the individual reagents may be separately housed. The kits may also include a means for enclosing the individual containers in relatively close confinement for commercial sale, e.g., a plastic box, in which instructions, packaging materials such as styrofoam, etc., may be enclosed.

In other embodiments, use of magnetic nanoparticles in the methods of the invention allows the construction of a magnetic cell-sorter that can differentiate and remove from solution only cells whose predetermined ion channels or receptors are active, e.g., in the presence of a particular compound such as a calcium channel activator. Moreover, calcium-sensitive nanoparticles can be targeted to mitochondria or to the nucleus, and calcium dynamics can be monitored in the proximity of these organelles. In such embodiments a delivery moiety that comprises a targeting sequence that directs uptake by an organelle can be used. For example, nuclear localization sequences are well known in the art.

The technology can also be extended from detection to control. In naturally occurring voltage-sensitive channels, for example, opening and closing events are driven by “paddles” formed by sub-domains within the channel. Such paddles carry positively-charged lysine groups and can be moved by a change in the external electric field. Such movements induce the opening and closing of the channel (37). Taking inspiration from this natural design, in one embodiment nanoparticles are appropriately attached to certain domains within an ion channel of interest: once such nanoparticles are in place, ion channel activity can be influenced or controlled by moving such nanoparticles using an externally applied magnetic field or an optical trap.

As mentioned above, in another embodiment, metal nanoparticles having different optical properties (e.g., color of scattered light) are attached to proteins of interest inside the cell (by means of the recognition designs described before). When such functionalized proteins come into contact with one another, the particles they carry change color. By monitoring such changes under dark-field microscopy, protein-protein interaction events can be studied inside living cells. An advantage over conventional FRET methods (currently used for this purpose) would be the non-bleaching nature of metal nanoparticles, which would allow observation for an indefinite length of time. This technology could also be used for drug-discovery applications, as it is amenable to high-throughput screening. For example, activation of a receptor or component of a signal transduction pathway could be detected by alterations in proximity of nanoparticles and change in plasmon resonance, and block or enhancement of this molecular movement or reaction, would be detected in response to potential therapeutic agents. Many receptors undergo conformational changes, e.g., changes in conformation of a receptor subunit or changes in the spatial relationship of two or more subunits to each other, or association with another cellular polypeptide, often in response to the binding of particular ligands. Such changes and associations can be detected and measured in accordance with the methods described above. The positioning of such proteins within the cell can also be controlled, by means of an external magnetic field or optical trapping, depending on the nature of the particles.

Nanosensors can also be attached to specific receptors residing in the cell membrane—on the side facing the exterior environment—to monitor or control their activity. Recombinant polypeptides, such as those described herein, may be used for a variety of additional purposes and may be detected using various other methods and assays, e.g., assays that make use of the recognition and/or target domain, in addition to, or instead of, the methods described herein.

In another embodiment, nanoparticles comprising a recognition domain that binds to a specific target domain of a polypeptide of interest are used to deliver an agent to a cell. For example, the nanoparticles can deliver the agent to a polypeptide of interest to which the nanoparticle becomes attached after its internalization. The target domain can be a heterologous target domain or a native target domain. In another embodiment the polypeptide comprises a target domain located in an extracellular portion of the polypeptide, in which case the nanoparticle does not need to be internalized. The target domain can be a heterologous target domain or a native target domain.

In either of these delivery methods, the agent can be entrapped or encapsulated in the nanoparticle (e.g., in the nanoparticle core, in a hollow cavity within the nanoparticle, in a coating layer, etc.), attached to the nanoparticle surface, or physically associated with the nanoparticle in any manner. The agent can, for example, be a known or candidate modulator of a polypeptide or protein of interest, e.g., an ion channel, GPCR, G protein, etc. By allowing specific and localized delivery to the vicinity of a protein of interest, these delivery methods enable a more specific evaluation of the effects of an agent on activity of the protein of interest and allow the artisan to distinguish the effect of the agent on the protein of interest from its effects on other cellular proteins.

In either of these delivery methods, following the binding of the nanoparticle recognition domain and the corresponding target domain, the agent is released from the nanoparticle. The release can occur by diffusion out of the nanoparticle or can occur as a result of nanoparticle erosion or breakdown (e.g., erosion or breakdown of a coating layer). In some embodiments a bond by which the agent is attached to the nanoparticle is cleaved, so that the agent is released. After release, the agent can diffuse to a nearby protein of interest. The polypeptide to which the nanoparticle recognition domain binds may, but need not be, the protein of interest or a subunit thereof. For example, the nanoparticle recognition domain may bind to an ion channel subunit, in which case the agent may be delivered to the ion channel or to another protein located in close proximity to the ion channel. The nanoparticle recognition domain may bind to a GPCR, in which case the agent may be delivered to the GPCR or to another protein located in close proximity to the GPCR, e.g, a G protein, an ion channel, a G protein target such as a downstream effector protein, etc.

### EXAMPLES

### Example 1

### Synthesis and Spectral Properties of Plasmon Resonant Gold Nanoparticles

Materials and Methods

Synthesis of gold nanoparticles. All glassware was cleaned in aqua regia and rinsed with ultrafiltered water before use. 200 mL of a 0.25 mM aqueous solution of HAuCl₄ were brought to a boil (plate temperature 280° C.) under constant, vigorous stirring (440 rpm). The pH of this solution was adjusted to 7 by addition of 120 μL of a 0.5 M aqueous solution of NaOH. 3.4 mL of a 50 mM solution of trisodium citrate were then added at a constant rate (approximately in 2 s). The solution slowly changed color: from transparent, to grey, purple and finally orange-red after circa ½ h. The heat was then turned off, but stirring was continued until the solution cooled to room temperature.

Modification of gold particles' surface for calcium sensitivity with MUA. A solution of 25 mM mercaptoundecanoic acid (MUA) was prepared by sonicating the powder for 10 min in a 50% water/ethanol solution, resulting in marked opalescence. The pH was adjusted to 7 with NaOH, after which the opalescence disappeared. 100 mL of a gold nanoparticle solution (prepared as described above) were brought to 60° C. under vigorous stirring. The pH was adjusted to 9 by addition of NaOH, after which the solution was brought to 500 μM mercaptoundecanoic acid by adding an aliquot of the previously prepared solution. The color immediately changed from red to slightly purple. Stirring at 60° C. was continued for 5 h. Prior to use as a calcium sensor, the excess MUA was removed from solution by centrifugation of the particles (25 min at 8,000 rpm on a bench centrifuge) and resuspension in water for three times. Other, e.g., longer, alkanethiols containing a carboxyl group could also be used in a similar manner.

Modification of gold particles' surface for calcium sensitivity with capping peptides. 15 nm Au particles were prepared according to the method described above (citrate reduction of gold chloride). Before proceeding to the coating step, the particle solution was brought to pH 7 by addition of NaOH. Coating of the particles with cysteine-terminated peptides was achieved by mixing the peptide of sequence CALNN (reduced using the disulfide reducing TCEP gel, from Pierce Biotechnology, Inc., according to the manufacturer's instructions) with the particle solution (typically 20 mL in a Falcon tube), which had been previously brought to 50° C. (to reduce oxygen concentration). After mixing and allowing the solution to stand for ˜1 h, it was brought to 20% PBS and incubated overnight at room temperature (increasing gradually the salt concentration allows peptides to pack more tightly on the particles' surface, due to charge screening). The minimum peptide concentration required for complete surface coverage (computed for a 2 nM solution of 15 nm particles and counting 2 peptides per square nanometer, as described by Fernig (28)) is predicted to be 3.4 μM. A 100-fold excess was found to be the optimal peptide concentration to use.

Results

TEM revealed the presence of nearly monodisperse Au, polycrystalline particles of spherical shape and diameter circa 15 nm when particles were coated with either MUA, peptides, or left uncoated. Uncoated particles showed a marked tendency to fuse. FIG. 14 shows the spectrum of a solution of 15 nm gold MUA-coated nanoparticles (red line) and its change when particles are induced to aggregate by the addition of calcium to the solution (blue line).

### Example 2

### Sensitivity and Specificity of Peptide-Functionalized Nanoscale Sensors to Calcium Ions

Gold nanoparticles (˜2 nM) were coated with a peptide displaying asparagine groups to the environment made and functionalized as in Example 1, using 34 mM peptide and the peptide CALNN (SEQ ID NO: 13). Following synthesis and functionalization, and after repeated washes in PBS by centrifugation and resuspension, nanoparticles were incubated in a solution containing either calcium or magnesium in water at a range of different concentrations. FIG. 16A shows the absorption spectrum of peptide-modified nanoparticles in the presence of various concentrations of calcium. The particles selectively chelate calcium ions, an event leading to their aggregation, which is readily detected by a spectral change in the presence of 1 mM Ca⁺⁺, relative to the spectrum at lower Ca⁺⁺ concentrations. Particle aggregation in the presence of Ca⁺⁺ is evident in FIG. 16B, left tube—note the colored particles at the bottom of the tube. When particles are exposed to the same concentration of Mg⁺⁺ (1 mM, right tube), they remain in solution and the absorption spectrum does not change (the image was taken 24 h after mixing the particles with the respective ions).

### Example 3

### Synthesis and Characterization of Calcium-Sensitive Polyacrylic Acid Coated Nanoparticles

Materials and Methods

Coating of gold nanoparticles with polyacrylic acid (PAA) for calcium sensitivity. Gold nanoparticles of diameter ˜15 nm were synthesized and coated with a monolayer of mercaptoundecanoic acid (MUA), according to the methods described in Example 1. After coating with MUA, particles carry a negative surface charge, due to the presence of carboxyl groups. In order to coat them with polyacrylic acid—also negatively charged—a layer of a positively charged polymer is first added. Particles were therefore coated with poly-diallyl-dimethylammonium-chloride (PDADMAC) by incubating them for ½ h in an aqueous solution of 10 mM NaCl, also containing 0.1% PDADMAC (20 KDa). The excess polymer was removed by centrifugation of the particles, removal of the supernatant and resuspension. In order to obtain the final layer of large-coiled polyacrylic acid, one needs to maximize the intra-chain charge repulsion of the polymer. This was achieved by adding the final coating in an environment of no ionic strength. Particles were suspended in ultra-filtered water, after which an aqueous solution of polyacrylic acid (100 KDa) was added, to bring the final concentration to 0.1%; incubation proceeded for ½ h. The excess polymer was removed by centrifugation and resuspension in TBS buffer.

Assessment of calcium sensitivity of PAA. The effect of calcium ions on the conformation of polyacrylic acid was tested by observing the effect of dropwise addition of a 10 mM aqueous solution of CaCl₂ to a 1% aqueous solution of polyacrylic acid

Assessment of calcium sensitivity of PAA-coated gold nanoparticles.

The sensitivity of the PAA-coated nanoparticles to the presence of calcium ions was tested by incubating the nanoparticles in TBS buffer and bringing the solution to 2 mM calcium. To demonstrate reversibility, the calcium chelator EDTA was then added to the solution. Absorption spectra were acquired prior to addition of calcium, in the presence of calcium but no EDTA, and following addition of EDTA.

Results

The effect of calcium on the conformation of polyacrylic acid is shown in FIG. 19. Panel A shows a 1% aqueous solution of polyacrylic acid, along with a schematic of its random coil conformation. Dropwise addition of a 10 mM aqueous solution of CaCl₂ induces immediate formation of a white precipitate (Panel B). Such precipitate is composed of clumped polymer chains: the carboxyl groups present along the polymer backbone chelate calcium ions and induce polymer collapse.

Coating nanoparticles with polymers can sometimes induce aggregation and precipitation. We adapted the layer-by-layer method developed by Caruso and coworkers (41), in which alternating positively- and negatively-charged polymer coatings allow the production of stable particles, avoiding aggregation, to coat gold nanoparticles with polyacrylic acid. This was achieved by tailoring the polymer flexibility (via ionic strength) to the particles' radius of curvature. For our purposes, we varied the ionic strength of the solution at different phases of the coating process, so as to obtain an external coat of polyacrylic acid with ample coils, which allowed collapse in the presence of calcium.

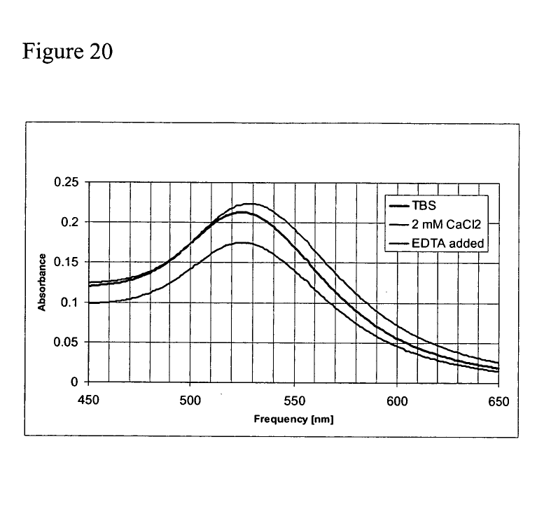

Exposure to calcium causes the peak plasmon resonance frequency of the PAA-coated nanoparticles to red-shift by circa 5 nm; reversibility is demonstrated by addition of the calcium chelator EDTA, following which the peak of the absorption spectrum returns to its previous value (FIG. 20).

### Example 4

### Conjugation of Nanoparticles with TAT Peptide and their Internalization by Cells

Materials and Methods

Conjugation of gold nanoparticles. Gold nanoparticles were synthesized as described in Example 1 and conjugated with BSA by mixing with an equal volume of a 1% BSA solution. Excess BSA was removed by centrifugation of the particles and resuspension in PBS. The cysteine-terminated TAT peptide n-CGGRKKRRQRRR-c (SEQ ID NO: 25) was custom synthesized using standard methods. In order to conjugate our nanoparticles with the TAT peptide, BSA-coated gold particles were conjugated with the cysteine-terminated TAT peptide using the commercially available cross-linker m-Maleimidobenzoyl-N-hydroxysuccinimide ester (MBS), from Pierce Biotechnology; the amine groups from the lysines in BSA were activated with MBS and reacted with the -SH group on the cysteine belonging to the TAT peptide.

In order to obtain TAT-conjugated nanoparticles that are also calcium-sensitive, the reduced TAT peptide (reduced using TCEP from Pierce) was incubated for 3 days with naked gold nanoparticles in TBS buffer. Excess peptide was removed by centrifugation and resuspension of the nanoparticles three times. The concentration of the solution was then brought to 300 μM MUA and left standing for 3 days, after which particles were washed three times by centrifugation and resuspension.

Cell culture and electroporation. Cells were cultured in DMEM supplemented with 10% FCS. Prior to electroporation, cells were trypsinized, washed twice with PBS and resuspended in OptiMEM at a density of 10⁷/ml. 600 μL of cell suspension were added to an electroporation cuvette with electrodes separated by 0.4 cm. 20-50 μL of nanoparticle suspension (approximately 10¹⁰ in number of 15 nm gold nanoparticles) were added to the cell suspension in the cuvette and incubated on ice for 15 min. The electroporation was carried out using a voltage of 0.27 kV and capacitor set at 960 μF. After electroporation, cells were cultured in complete medium and allowed to recover overnight before analysis by dark-field microscopy.

Results

FIG. 10 shows dark-field microscopy images of nanoparticle internalization by HEK cells. In panel (A), HEK cells were incubated for 5 min with TAT-conjugated gold nanoparticles (bright spots) with no electroporation. The two bright spots are most probably particles engaged onto the cell surface. The cells in panel A were treated in the same manner except that cells were subjected to electroporation in the early stage of incubation. Particles were readily internalized in this case.

FIG. 11 shows electron microscopy images of COS cells containing internalized nanoparticles. Gold nanoparticles (15 nm diameter) were either electroporated (left) or endocytosed (right) by COS cells. In the first case, many particles were found well-dispersed throughout the cell cytosol (seen in proximity to the Golgi apparatus in the left image). Endocytosed particles, on the other hand, were mostly trapped within endosomes (right image).

Absorption spectra have been obtained from a sample of COS cells with internalized nanoparticles (data not shown). The instrument was blanked using cells that did not contain nanoparticles. Different nanoparticle concentrations, ranging from 10⁵ particles/cell to 10⁶ particles/cell, were employed.

### Example 5

### Calcium Sensing Inside Living Cells Using a Fret-Based Calcium Sensor

Quantum dot nanoparticles with an absorption peak at ˜405 nm and an emission peak at ˜580 nm (“Birch Yellow, Type 2”, Evident Corp.) were internalized by COS cells that had been previously loaded with a calcium-sensitive dye with absorption ˜580 nm, emission ˜620 nm (Calcium Crimson; Invitrogen N. C-3018). Dye loading was performed according to the directions of the manufacturer, and electroporation was used to enhance nanoparticle uptake. Live cells were illuminated with a 405 nm laser and emission from the sample was collected through a narrow bandgap filter centered around 620 nm. Upon addition of thapsigargin (a compound that induces calcium entrance into the cytosol from internal stores), flashes of light (orange dots) were detected only in the immediate vicinity of the nanoparticles. Since the calcium-sensitive dye cannot be excited by the 405 nm laser, their activation is due to FRET from the nanoparticles. The image is a snapshot from a movie taken at mid-plane section of a cell via confocal microscopy.

### Example 6

### Construction of Engineered Ion Channel Subunits and Characterization of their Expression and Functional Activity

Materials and Methods

Plasmid construction. Plasmids containing ion channel subunits fused to either of two different recognition domains (barstar or a coiled-coil heptad repeat) were generated as described below. The recognition domains were inserted at the N-terminus of the ion channel subunit. In order to allow characterization of the engineered ion channel subunits using fluorescence microscopy, a sequence encoding enhanced GFP (EGFP) was also included in the constructs. The EGFP coding sequence was positioned between the coding sequence for the recognition domain and the coding sequence for the ion channel subunit.

Generation of EGFP-barstar and EGFP coiled-coil constructs. The heptad repeat (EVSALEK)₄ was fused to the N-terminus of EGFP by overlap PCR using the following 3 primers as forward primers and the vector EGFP-C1 (Clontech) to provide the EGFP template:

| | |
|---|---|
| 5′-CCCGCTAGCGCCACCATGAAGGTAAGCGCCTTC | (SEQ ID NO:26) |
| AAGGAGAAAGTGTCCGCACTGAAAGAA-3′ | |
| 5′-AAGTGTCCGCACTGAAAGAAAAAGTTTCTGCAC | (SEQ ID NO:27) |
| TGAAGGAAAAGGTCTCGGCACTCAAG-3′ | |
| 5′-AAGGTCTCGGCACTCAAGGAAGGTGGTGGCATG | (SEQ ID NO:28) |
| GTGAGCAAGGGCGAGG-3′ | |

and the following reverse primer:

| | |
|---|---|
| 5′-GGTTCAGGGGGAGGTGTG-3′ | (SEQ ID NO:29) |

The PCR product obtained coded for the EGFP protein, including the heptad repeat (EVSALEK)₄ at the N-terminus of EGFP, with additional NheI and EcoRI restriction sites at the 5′ and 3′ end, respectively. The PCR product was cloned into the EGFP vector backbone using NheI and EcoRI.

The sequence encoding Barstar was fused to the N-terminus of EGFP by first amplifying barstar (from a plasmid encoding barstar, obtained from A. Plückthun, U. of Zurich) with the following primers:

PCR Reaction A

| | |
|---|---|
| 5′-GATCCGCTAGCCACCATGAAAAAAGCAGTCATT | (SEQ ID NO:30) |
| AACGGG-3′ | |
| 5′-CAGCTCCTCGCCCTTGCTCACCATAGAAAGTAT | (SEQ ID NO:31) |
| GATGGTGATGTCGCA-3′ | |

and amplifying EGFP with the following primers:

PCR Reaction B

| | |
|---|---|
| 5′-TGCGACATCACCATCATACTTTCTATGGTGAGC | (SEQ ID NO:32) |
| AAGGGCGAGGAGCTG-3′ | |
| 5′-GGTTCAGGGGGAGGTGTG-3′ | (SEQ ID NO:33) |

The PCR product encoding the barstar-EGFP fusion protein was obtained by using PCR products A and B as templates and primers 1 and 4 as amplification primers. The PCR product was cloned into the EGFP-C1 vector backbone using NheI and EcoRI restriction sites resulting in constructs that encode EGFP with either the heptad repeat or barstar fused in frame at the N terminus. These constructs are referred to as EGFP-cc and EGFP-barstar.

Cloning of TRPV3, TRPV1 and P2X2 into the EGFP-cc and EGFP-barstar vectors. The multiple cloning site (MCS) 3′ of the EGFP-C1 vector is not affected by the heptad repeat or barstar insertion described above, i.e., the restriction sites at the 3 prime end of EGFP are identical to the original EGFP-C1 clone. This MCS was used to clone each of three different ion channel subunits in-frame with the EGFP-heptad repeat module or EGFP-barstar module that had been inserted into the EGFP-C1 vector. Mouse TRPV3 cDNA was amplified either from total mouse cDNA or from plasmids containing the appropriate subunit sequence by PCR using the following primers:

| | |
|---|---|
| 5′-AGGCAAGCTTCGATGAATGCCCACTCCAAG-3′ | (SEQ ID NO:34) |
| and | |
| 5′-AATTGGATCCCTACACCGACGTTTCTGG-3′. | (SEQ ID NO:35) |

The resulting PCR product was digested using the restriction enzymes HindIII and BamHI and cloned into the EGFP-cc and EGFP-barstar vectors in frame with the EGFP-cc and EGFP-barstar modules using HindIII and BamHI restriction sites.

Rat TROV1 cDNA was Amplified Using the Following Primers:

| | |
|---|---|
| 5- | |
| GATGTCGACAGATGGAACAACGGGCTAGC-3′ | (SEQ ID NO:36) |
| 5′- | |
| TGCAGAATTCTTATTTCTCCCCTGGGAC-3′ | (SEQ ID NO:37) |

The resulting PCR product was digested using the restriction enzymes SalI and EcoRI and cloned into the EGFP-cc and EGFP-barstar vectors in frame with the EGFP-cc and EGFP-barstar modules using XhoI and EcoRI restriction sites.

Rat P2X2 cDNA was Amplified by PCR Using the Primers

| | |
|---|---|
| 5′-AATTAGATCTACCATGGTCCGGCGCTTGGCCC | (SEQ ID NO:38) |
| G-3′ | |
| 5′-TTTGTCGACTCAAAGTTGGGCCAAACCTTTG-3′ | (SEQ ID NO:39) |

The resulting PCR product was digested using the restriction enzymes BglII and SalI and cloned in frame into the EGFP-cc and EGFP-barstar vectors in frame with the EGFP-cc and EGFP-barstar modules using BglII and SalI restriction sites to generate constructs containing the following components in the N- to C-terminal direction, (i) a recognition domain (either the heptad repeat or barstar); (ii) EGFP; (iii) TRPV3, TRPV1, or P2X2.

Transient transfection of COS and HEK cells. COS or HEK cells were transfected using Lipofectamine 2000 (Invitrogen) according to the manufacture's protocol. In brief, 2 μg of the respective DNA plasmid were incubated for 30 min with 6 μl of Lipofectamine in 200 μl of Optimem medium (Gibco) at room temperature. Cells were incubated with the DNA-Lipofectamine complex for 3 h at 37 degrees Celsius in 500 μl Optimem medium. Afterwards the medium was replaced with DMEM containing 10% FCS. Cells were assayed for expression and activity of the engineered ion channels 24-48 h after transfection.

Characterization of expression of engineered ion channel subunits. Expression of the engineered TRPV3-cc and TRPV3-barstar ion channel subunits in HEK cells after transient transfection was examined by fluorescence microscopy to detect EGFP fluorescence. Cells were also immunostained and examined by fluorescence microscopy. The FLAG epitope had been previously inserted into the extracellular domain of TRPV3, and the FlagM2 antibody (Sigma) was used to detect membrane localized channels.

Characterization of functional activity of engineered ion channel subunits. Whole-cell patch clamp recordings were taken from HEK293T cells transiently transfected with the construct of choice. The voltage clamp recordings were made using an Axopatch 200A amplifier, and controlled by a Digidata 1320 and pClamp 9.2 software. The cells were voltage clamped with a holding potential of 0 mV and exposed to a voltage ramp from −100 mV to +100 mV every 4 seconds. Recordings were made with External Solution, which is standard modified Tyrode's Solution (22° C.): 150 mM NaCl, 5 mM KCl, 1 mM MgCl₂, 2 mM CaCl₂, 20 mM HEPES, 10 mM Glucose, pH 7.4 (NaOH adjusted), osmolality 311. 100 μM 2APB solution was made by adding 100 μM 2APB to standard external solution. Internal pipette solution is standard internal solution (22° C.): 147 mM Cs⁺, 120 mM Methane-Sulfonate, 8 mM NaCl, 10 mM EGTA, 2 mM Mg-ATP, 20 mM HEPES, pH 7.2 (CsOH adjusted).

Results

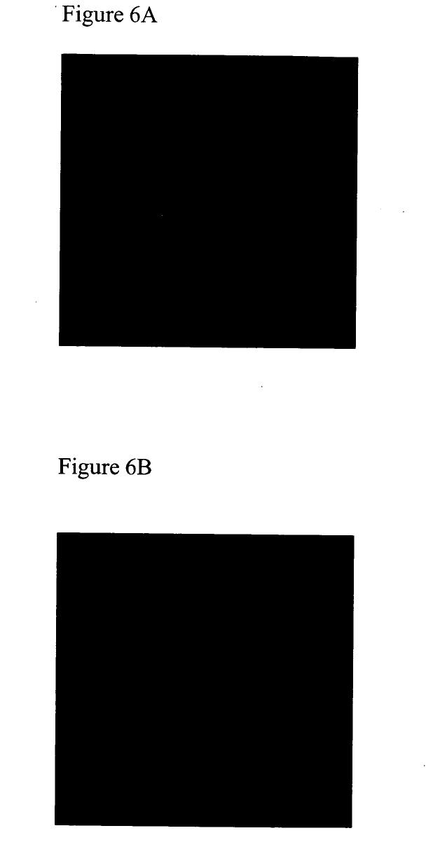

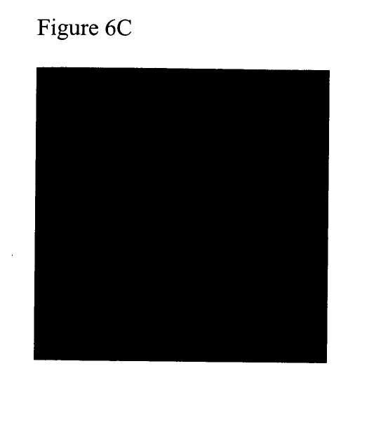

To evaluate expression and localization of the engineered TRPV3 ion channel subunits and assembly and localization of ion channels containing them, cells were transiently transfected with constructs encoding either TRPV3-EGFP or TRPV3-EGFP-cc. As shown in FIGS. 6A-6C, EGFP fluorescence is evident in cells transfected with either TRPV3-EGFP-cc (FIG. 6A), TRPV3-EGFP-barstar (FIG. 6B), or a construct encoding TRPV3 with EGFP at its N terminus but lacking a recognition domain (TRPV3-EGFP) (FIG. 6C). These results indicate that the engineered ion channel subunits are expressed. Immunostaining with an antibody that binds to an extracellular epitope of TRPV3 (inserted FLAG tag) indicated that the engineered ion channel subunits were inserted into the plasma membrane and assumed the proper topology.

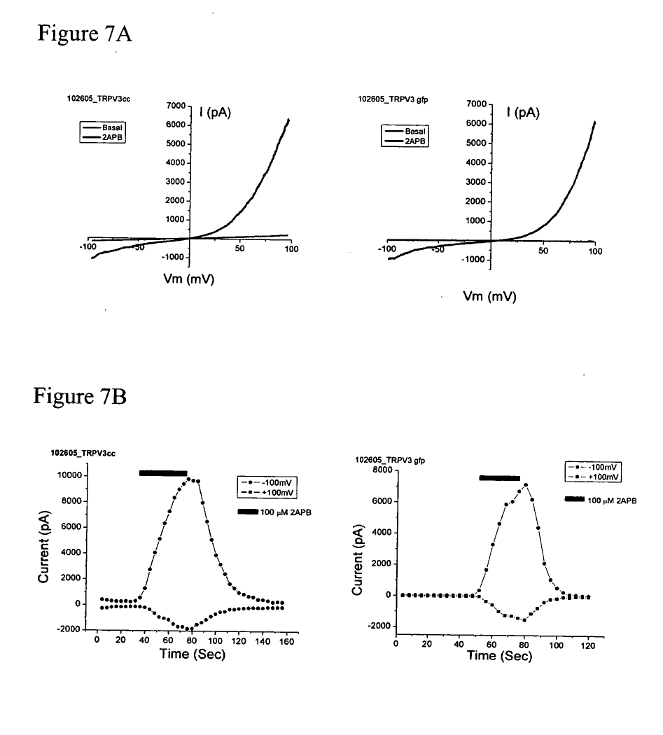

To evaluate the functional activity of ion channels channels containing engineered TRPV3 subunits, whole-cell patch clamp recordings were made from cells that had been transiently transfected with constructs encoding TRPV3-EGPF-cc or TRPV3-EGFP. FIG. 7A shows UV relationships before and after application of 100 μM 2APB to cells expressing TRPV3-EGFP-cc (left) and cells expressing TRPV3-EGFP (right). FIG. 7B shows a timecourse of application of 100 μM 2APB to cells expressing TRPV3-EGFP-cc (left) and TRPV3-EGFP (right). Cells transfected with either TRPV3-EGFP or TRPV3-cc responded similarly to application of 100 μM 2APB and this effect was quickly washed out.

These results demonstrate that TRPV3-cc is functionally expressed in transiently transfected HEK293T cells. Channels containing TRPV3-cc are robustly activated and behave in a qualitatively similar manner to channels containing TRPV3-EGFP. Previous results had shown that channels containing TRPV3-EGFP behaved in a qualitatively similar manner to wild type channels.

### Example 7

### Production of Barnase Mutants

Materials and Methods

Generation of Haemaglutinin-Barnase, GST-Barnase, Hamaglutinin-BarnaseHis-102-Ala and GST-BarnaseHis-102-Ala constructs. The sequence encoding for barnase with BamHI and XhoI restriction sites at the 5′ and 3′ end was amplified using the primers:

| | |
|---|---|
| 5′-AAAGGATCCGCCACCATGGCACAGGTTATCAAC | (SEQ ID NO:40) |
| ACGTTTG-3′ | |
| 5′-TTCCCTCGAGTCAGCAACAGTGATGGTGATGAT | (SEQ ID NO:41) |
| GGTGCGG-3′ | |

The PCR product was cloned in-frame to the 3′ end of Haemaglutinin (HA) (pcDNA6 vector (Invitrogen)). The HA tag had been previously introduced into the vector using the NheI and HindIII restriction sites and GST epitope tag (pGEX −4T-1 vector; Amersham Bioscience) introduced using BamHI and XhoI restriction sites such that the tags were positioned at the N-terminus of barnase. The histidine at position 102 in barnase was mutated to alanine by site-directed mutagenesis using the following primers with the Haemaglutinin-Barnase and 6X-GST-Barnase constructs as templates.

| | |
|---|---|
| 5′-TACAAAACAACGGACGCTTATCAGACCTTTAC | (SEQ ID NO:42) |
| A-3′ | |
| 5′-TGTAAAGGTCTGATAAGCGTCCGTTGTTTTGT | (SEQ ID NO:43) |
| A-3′ | |

GST-tagged barnase was purified form E. coli lysate using a Glutathione sepharose column according to the manufactures protocol. The GST tag was cleaved off using thrombin following the manufactures protocol.

Results

In order to minimize the possibility that presence of an engineered polypeptide containing barnase inside cells would be toxic, a barnase mutant in which an active site residue (His 102) was mutated to Ala was produced. Barnase and barstar were readily expressed and purified. However, initial experiments suggested that the barnase variant in which His 102 was altered to Ala did not bind efficiently bind barstar.

Further Experiments

In further experiments, additional barnase variants are generated and tested for RNase activity and for their ability to bind barstar. In particular, variants in which His 102 is mutated to an amino acid other than Ala are produced, and variants in which one or both of the other two active site residues, Glu-73 and Lys-27 (42) is mutated either to Ala or to another amino acid are tested. A variant that displays low or undetectable RNase activity and retains ability to bind barstar is identified. If none of these barnase variants proves to have both a desirably low level of RNase activity and a desirably high ability to bind to barstar, then additional residues located near one of or more of the active site residues are mutated either individually or in combination with mutation of one or more active site residues. Systematic generation and testing of barnase variants either by site-directed mutagenesis or random mutagenesis results in identification of a barnase variant having a desirably low level of RNase activity and a desirably high ability to bind to barstar. If necessary each residue in barnase is mutated either individually or in combination with mutation of one or more active site residues until a suitable variant is found. Barstar and the identified barnase variant are employed as a recognition domain/target domain pair.

In further experiments, barstar variants are generated and tested for their ability to bind the inactive barnase variant in which His 102 is altered to Ala. Selection of residues to mutate is guided by knowledge of the structure of the barnase/barstar complex. A barstar variant able to bind with high affinity (e.g., K_{d}<10⁻⁶ M, preferably <10⁻⁷ M) to the inactive barnase variant is identified. The barstar and barnase variants are employed as a recognition domain/target domain pair.

### Example 8

### Specific Binding of Functionalized Nanoparticles and a Polypeptide of Interest Via Interaction of Complementary Coiled-Coil Peptides

Nanoparticle synthesis and conjugation. Dye-doped silica nanoparticles containing the fluorescent dye tris (2,2′-bipyridine) dichlororuthenium (II) hexahydrate) (Ru(bpy)) were synthesized following the reverse microemulsion procedure described by Tan and coworkers (35). These particles are approximately 40 nm in diameter and can be dispersed in de-ionized water. To impart biocompatibility (stability in PBS and other physiological buffers), particles were coated with bovine serum albumin (BSA) by mixing the nanoparticles in aqueous solution with an equal volume of freshly prepared BSA solution (1% weight in water). The mixture was incubated at room temperature for 1 h, after which it was brought to a standard PBS concentration by addition of an appropriate amount of concentrated PBS solution (10× PBS) and left standing overnight at room temperature. Excess BSA was then removed by repeated cycles of centrifugation (12 min at 9,500 rpm), removal of the supernatant and resuspension in PBS (a step involving bath sonication for a few minutes).

Conjugation of the nanoparticles to the recognition domain peptide CGG-(KVSALKE)₃, (SEQ ID NO: 44) which contains a three amino acid spacer (CGG) followed by three copies of the KVSALKE (SEQ ID NO: 1) heptad sequence (cc, custom synthesized by the MIT biopolymer lab) was achieved using commercial crosslinking chemistry. Amine groups on the surface of BSA-coated nanoparticles (there are 35 lysine groups accessible at the surface of each BSA molecule) were activated by transferring the nanoparticles to a solution of 10 μM or 100 μM freshly prepared MBS (an amine-to-thiol crosslinker, available from Pierce Biotechnology) in PBS. After 20 min incubation, nanoparticles were washed by centrifugation and resuspension in a conjugation buffer (PBS at pH 7 and 1 mM EDTA). Since the recognition peptide contains a cysteine (chosen for conjugation), reduction of possible disulfide bonds was performed (using TCEP reducing gel, Pierce Biotechnology) before addition to the activated nanoparticles. Conjugation was completed by adding the reduced peptide at a concentration of 10 μM or 100 μM to nanoparticles activated with MBS at the same respective concentrations. After peptide addition, nanoparticles were incubated overnight in a gently rotating tube at 4° C., after which they were washed 3 times in PBS.

HEK 293T cells were transiently transfected with a construct encoding either EGFP or the EGFP-cc construct containing (EVSALEK)₄ (SEQ ID NO: 4) fused to the N-terminus of EGFP as described in Example 6. 2×10⁶ cells were lysed in 200 μl Lysis buffer (PBS, pH7.4 with 1% Triton-X100) and afterwards diluted with 800 μl PBS to obtain 1 ml of cell lysate with 0.2% Triton-X100. Pull-down experiments were performed by incubating 100 μL of nanoparticles with 900 μL of cell lysate for one hour at 4° C. (with gentle rotation). Particles were then centrifuged (12 min at 9,500 rpm) and the supernatant removed. After brief washing of the pellet with 100 μL of PBS, particles were resuspended by sonication in 50 μL of PBS and the resulting solution analyzed by Western blot using antibodies to EGFP (Covance) according to standard methods.

Results

Pull-down experiments were performed to assess the ability of the complementary heptad repeats to mediate binding of the functionalized nanoparticles (NP-cc) to the EGFP-cc polypeptide. Briefly, lysates made from cells expressing either EGFP or EGFP-cc (where cc=(EVSALEK)₄) (SEQ ID NO: 4) were incubated in the presence of nanoparticles that had been functionalized with a complementary heptad repeat. The nanoparticles were recovered, and proteins bound to them were analyzed for presence of EGFP.

FIG. 8, left panel, shows a Western blot in which lysates from cells expressing either EGFP (left lane) or EGFP-cc (right lane) were probed to detect presence of EGFP. The band at the size of EGFP in the right lane may represent EGFP-cc in which the cc has been cleaved off. The right panel of FIG. 8 shows Western blot analysis of proteins recovered from the pellets to detect presence of EGFP. The left lane shows analysis of proteins recovered from functionalized nanoparticles that had been incubated in lysate of cells expressing EGFP-cc. Note the strong band at the position expected for EGFP-cc. The right lane shows analysis of proteins recovered from unfunctionalized nanoparticles that had also been incubated in lysate of cells expressing EGFP-cc. The faint band at the position expected for EGFP-cc represents a small amount of nonspecific binding to the nanoparticles. Based on visual inspection, nonspecific binding of EGFP-cc to the unfunctionalized nanoparticles appears to be orders of magnitude less than the amount of specific binding of EGFP-cc to nanoparticles functionalized with a complementary heptad repeat.

These results confirm the binding of nanoparticles (NP-cc) functionalized with a heptad repeat recognition domain to a polypeptide containing the complementary target domain under conditions at least as stringent as those that would be expected to exist inside cells. Experiments using different amounts of CGG-(KVSALKE)₃ (SEQ ID NO: 44) conjugated to nanoparticles confirmed that the amount of EGFP-cc pulled down by NP-cc is proportional to the number of heptad repeat peptides conjugated to the nanoparticles (data not shown).

### Example 9

### Specific Binding of Functionalized Nanoparticles to an Engineered Ion Channel Via Interaction of Complementary Coiled-Coil Peptides

Materials and Methods

Nanoparticle synthesis and conjugation. These were performed as described in the previous example.

Pull-down experiments. These were performed essentially as described in the previous example except that (i) two different ratios of peptides/nanoparticle (10 μM or 100 μM peptide) were used to test their effect on binding ability; and (ii) lysates were made from cells that expressed engineered ion channel subunits TRPV3-EGFP-cc and TRPV3-EGFP, which are described in Example 6.

Results

Pull-down experiments were performed to assess the ability of the complementary heptad repeats to mediate binding of the functionalized nanoparticles to engineered TRPV3 ion channel subunits containing either EGFP-cc or EGFP at the N-terminus of the ion channel sequence. Briefly, lysates made from cells expressing either TRPV3-EGFP or TRPV3-EGFP-cc (where cc=(EVSALEK)₄) (SEQ ID NO: 4) were incubated in the presence of nanoparticles that had been functionalized with a complementary heptad repeat. The nanoparticles were recovered, and proteins bound to them were analyzed for presence of EGFP.

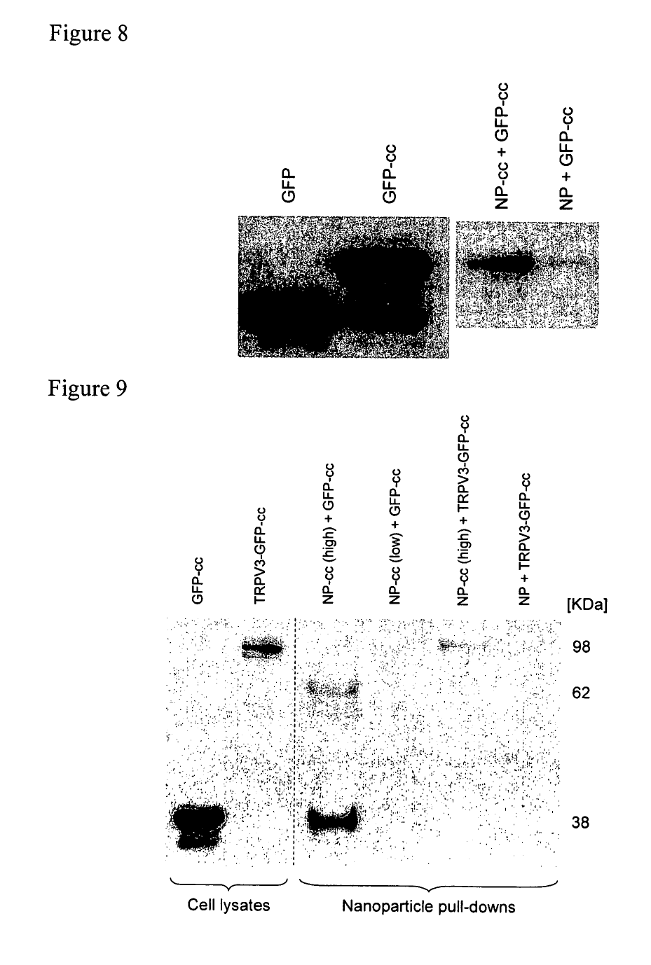

FIG. 9 shows a Western blot in which lysates from cells expressing either EGFP-cc or TRPV3-EGFP-cc or proteins recovered from nanoparticles that had been incubated in such lysates were probed with an antibody that recognizes EGFP. The leftmost two lanes show detection of EGFP-cc or TRPV3-EGFP-cc in lysates from cells that express either EGFP-cc (lane 1, band at ˜38 kD) or TRPV3-EGFP-cc (lane 2, band at ˜98), respectively. Lane 3 (to the right of the dotted line) shows presence of EGFP-cc (lane 3) in proteins recovered from functionalized nanoparticles that had been incubated in lysate of cells expressing these two polypeptides. Lane 5 shows presence of EGFP-cc (lane 5) or TRPV3-EGFP-cc (lane 6) respectively in proteins recovered from functionalized nanoparticles that had been incubated in lysate of cells expressing these two polypeptides. The nanoparticles used in these incubations were conjugated with 100 μm peptide. Similar incubations using nanoparticles conjugated with 10 μm peptide were also performed. The lower concentration did not result in detectable pull-down of EGFP-cc by NP-cc (lane 4) under the conditions tested. A very faint band representing only a small amount of pull-down of TRPV3-EGFP-cc is evident in lane 6.

These results confirm the binding of nanoparticles (NP-cc) functionalized with a heptad repeat recognition domain to an ion channel subunit containing the complementary target domain under conditions at least as stringent as those that would be expected to exist inside cells.

To test the functionality of this recognition motif in vivo, the dye-labeled nanoparticle nanoscale sensors are delivered into live cells expressing ion channels engineered to specifically bind them (TRPV3-EGFP-cc). Since Ru(bpy) produces a red fluorescence signal while EGFP produces a green fluorescence signal, both the nanoparticles and engineered ion channel subunits can be detected. Colocalization of the ion channel subunits and nanoparticles, with appropriate controls to account for possible spectral bleed through, would suggest that internalized nanoparticles reach and bind to their target ion channel within living cells.

### Example 10

### Generation of Stable Cell Lines that Inducibly Express Ion Channel Subunits mTRP3-EGFP-cc, TPV1-EGFP-cc, or P2X2-EGPF-cc

Inducible, stable COS cell lines expressing the engineered ion channel subunits described in Example 6 were generated using the T-REX system (Invitrogen). In brief, COS-7 cells were transfected in 3.5 cm dishes with 2 μg of the TET repressor gene in the pcDNA6/TO vector according to the manufacturer's instructions. Cells stably expressing the TET repressor protein were selected with 10 μg/ml Blasticidin in DMEM with 10% FCS. Single clones were isolated after 2 weeks of selection and the highest expressing clone was identified. Constructs TRPV3-EGFP-cc, TRPV1-EGFP-cc or P2X2-EGFP-cc were transfected into this clone following the manufacturer's protocol, and cells stably expressing either TRPV3-EGFP-cc, TRPV1-EGFP-cc or P2X2-EGFP-cc were isolated by selection in 250 μg/ml Zeocin in DMEM with 10% FCS. After 2 weeks of selection single clones are isolated and characterized.

### Example 11

### Synthesis of Silica Nanoparticles with a Calcium-Sensitive Dye Embedded in the Silica Matrix

Methods for producing monodisperse silica nanoparticles that rely on the hydrolysis of tetraethoxyorthosilicate (TEOS) catalyzed by ammonia in water/ethanol mixtures were developed by Stober (43). Van Blaaderen and Vrij developed an extension of the method that allows covalent embedding of fluorescent molecules within the silica matrix (44). Their strategy relies on covalent binding of the fluorescent molecule to a variant of TEOS before hydrolysis, so that the silica matrix is formed using building blocks that are already functionalized with a fluorescent molecule when the reaction starts.

Our approach utilizes the covalent binding of a thiol-reactive calcium-sensitive molecule to mercaptopropyltrimethoxysilane (MPTS, a silane that possesses a thiol group available for functionalization). MPTS or another silane possessing a thiol group is used instead of TEOS (or in combination with it) as the starting material for production of nanoparticles through hydrolysis (either via Stober synthesis or via a water-in-oil microemulsion method (35)) to produce a population of silica nanoparticles that carry within their matrix a covalently attached, ion-sensitive fluorescent molecule. Suitable thiol-reactive, Ca⁺⁺-sensitive fluorescent molecules are commercially available. For example, Ca⁺⁺-sensitive, thiol-reactive fluo-4 iodoacetamide (Invitrogen, catalog N. F36200), can be used. Following synthesis, the surface of these nanoparticles is functionalized with recognition domains such as those described above, allowing attachment of the nanoparticle to any ion channel of interest.

### Example 12

### Synthesis of Mesoporous Silica Nanoparticles with a Calcium-Sensitive Dye Covalently Attached to the Pore Walls

Mesoporous silica nanoparticles can be synthesized by hydrolysis of TEOS (or its derivatives) in the presence of surfactants. The size of the particles and their pores can be tuned by changing the molar ratio of reactants during synthesis.

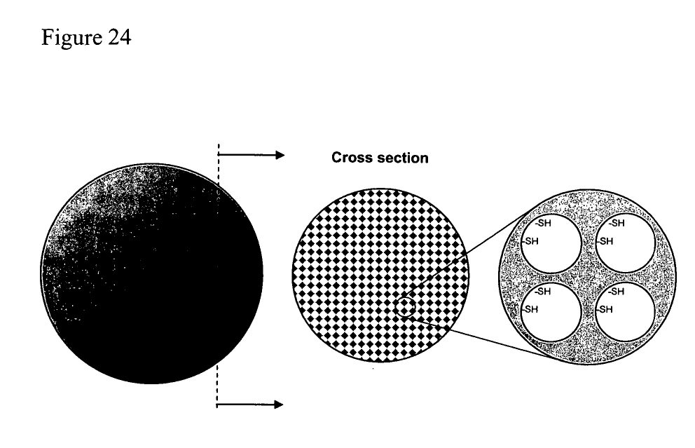

Thiol-derivatized nanoparticles composed of an ordered, silica, mesoporous silica matrix characterized by well-organized nano-pores of cylindrical shape (nano-channels) are synthesized by dropwise addition of TEOS to a n-Cetyltrimethylammonium bromide (CTAB) solution, followed by dropwise addition of 3-mercaptoporopytrimethoxysilane (MPTMS) as described (45). The synthesis method results in mesoporous silica nanoparticles with thiol groups lining the interior surface of the channels, as shown schematically in FIG. 24. To synthesize mesoporous silica nanoparticles with a Ca⁺⁺-sensitive dye covalently attached to the pore walls, a Ca⁺⁺-sensitive, thiol-reactive fluorescent dye (such as fluo-4 iodoacetamide, as described in Example 11) is allowed to react with the thiol groups lining the channels to form a covalent linkage. Following synthesis, the surface of these nanoparticles is functionalized with recognition domains such as those described above, allowing attachment of the nanoparticle to any ion channel of interest.

### Example 13

### Detecting Activity of an Engineered TRPV3 Ion Channel Using an Attached Nanoscale Sensor

A stable cell line (TRPV3-EGFP-cc-st) that inducibly expresses a modified TRPV3 ion channel subunit containing a (EVSALEK)₄ recognition domain is generated as described in Example 10. The cells are cultured under inducing conditions, resulting in synthesis of the engineered ion channel subunit. Silica nanoparticles containing quantum dots with an absorption peak at ˜405 nm and an emission peak at ˜580 nm are functionalized with a heptad repeat recognition domain complementary to (EVSALEK)₄ (SEQ ID NO: 4) (i.e., (KVSALKE)₃) (SEQ ID NO: 3) and the TAT peptide described in Example 4. The functionalized nanoparticles are incubated with TRPV3-EGFP-cc-st cells that have been previously loaded with a calcium-sensitive dye (Calcium Crimson; absorption ˜580 nm, emission ˜620 nm). The nanoparticles are internalized by TRPV3-EGFP-cc-st cells.

The cells are plated in wells of a 384-well multiwell dish. Different compounds from a compound library are dispensed into each well. Cells are illuminated using a 405 nm laser and emission from the sample is collected through a narrow bandgap filter centered around 620 nm. Flashes of light (orange dots) coming from cells in a well indicate TRPV3 ion channel activity in the cells in that well. A difference, e.g., a statistically significant difference, in the amount of light coming from any individual well relative to the amount of light coming from a control well to which no compound is added indicates that the added compound is an activator of the TRPV3 channel if the amount of light in the presence of the compound is greater than in its absence. A difference, e.g., a statistically significant difference in the amount of light coming from any individual well relative to the amount of light coming from a control well to which no compound is added indicates that the added compound is an inhibitor of the TRPV3 channel if the amount of light in the presence of the compound is less than in its absence.

Additional stable cell lines containing engineered ion channel subunits that contain the cc recognition domain but do not contain EGFP are also generated using standard recombinant DNA methods such as those described above. The cell lines are also used to screen for compounds that activate or inhibit ion channels containing the engineered subunits.

### Example 14

### Detecting Activity of an Engineered P2X2 Ion Channel Using an Attached Nanoscale Sensor

A stable cell line (P2X2-cc-st) that inducibly expresses a modified P2X2 ion channel subunit containing a heptad repeat (EVSALEK)₄ recognition domain is generated in a similar manner to that described in Example 10 using a construct that encodes P2X2-cc, with the heptad repeat at the N-terminus of P2X2. The cells are cultured in the absence of tetracycline to induce synthesis of the engineered ion channel subunit. Mesoporous silica nanoparticles containing a covalently attached calcium-sensitive dye (fluo-4) are synthesized as described in Example 12 and functionalized with a heptad repeat recognition domain complementary to (EVSALEK)₄ (SEQ ID NO: 4) (i.e., (KVSALKE)₃) (SEQ ID NO: 3) and the TAT peptide described in Example 4. The functionalized nanoparticles are incubated with P2X2-cc-st cells for 5 minutes, following which the cells are subjected to electroporation to enhance nanoparticle internalization.

After electroporation cells are plated in wells of a 384-well multiwell dish. Different compounds from a compound library are dispensed into each well. Cells are illuminated at the excitation frequency of fluo-4 using a laser and emission from the sample is collected through a narrow bandgap filter centered around the emission frequency of fluo-4. A fluorescent signal coming from cells in a well indicates P2X2 ion channel activity in the cells in that well.

A difference, e.g., a statistically significant difference, in the signal coming from any individual well relative to the signal coming from a control well to which no compound is added indicates that the added compound is an activator of the P2X2 channel if the signal in the presence of the compound is greater than in its absence. A known activator of P2X2 channels, ATP, is used as a positive control.

In additional experiments, ATP is added to each well after addition of the test compound. A difference, e.g., a statistically significant difference, in the signal coming from any individual well relative to the signal coming from a control well to which no test compound is added, but to which ATP has been added, indicates that the added compound is an inhibitor of the P2X2 channel if the signal in the presence of the compound is less than in its absence. For example, the test compound may be a blocker of the P2X2 channel, which prevents activation of the channel by ATP.

### Equivalents and Scope

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,”, “an” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nanoparticle type, characteristic, size, material composition, etc., any target or recognition domain, any delivery moiety, any method of sensing ion channel or receptor activity, any particular analyte, any particular ion channel or receptor or subunit thereof, any particular molecule or component employed in a nanosensor, etc.), can be excluded from any one or more claims, for any reason, whether or not related to the existence of prior art.

### REFERENCE LIST

1. Sakmann, B. and Neher, E. Patch-clamp techniques for studying ionic channels in excitable membranes. Annu. Rev. Physiol. 46, 455 (1984).
2. Berridge, M. J., Lipp P. and Bootman, M. D. The versatility and universality of calcium signaling. Nat. Rev. Mol. Cell. Bio. 1, 11 (2000).
3. Berridge, M. J., Bootman, M. D. and Roderick, H. L. Calcium signaling: dynamics, homeostasis and remodeling. Nat. Rev. Mol. Cell. Bio. 4, 517 (2003).
4. Seglen, P. O., Grinde, B. and Solheim, A. E. Inhibition of the lysosomal pathway of protein degradation in isolated rat hepatocytes by ammonia, methylamine, chloroquine and leupeptin. Eur. J. Biochem. 95, 215 (1979).
5. Drin, G., Cottin, S., Blanc, E., Rees, A. R. and Temsamani, J. Studies on the internalization mechanism of cationic cell-penetrating peptides. J. Biol. Chem. 278, 31192 (2003).
6. Green, M. and Loewenstein, P. M. Autonomous functional domains of chemically synthesized human immunodeficiency virus tat trans-activator protein. Cell 55, 1179 (1988).
7. Frankel, A. D. and Pabo, C. O. Cellular uptake of the tat protein from human immunodeficiency virus. Cell 55, 1189 (1988).
8. Lewin, M., Carlesso, N., Tungl, C-H, Tang, X-W, Cory, D., Scadden, D. T. and Weissleder, R. Tat peptide-derivatized magnetic nanoparticles allow in vivo tracking and recovery of progenitor cells. Nat. Biotech. 18, 410 (2000).
9. Wadia, J. S., Stan, R. V. and Dowdy, S. F. Transducible TAT-HA fusogenic peptide enhances escape of TAT-fusion proteins after lipid raft macropinocytosis. Nat. Med 3, 310 (2004).
10. Won, J., Kim, M., Yi, Y-W, Kim, Y. H., Jung, N. and Kim, T. K. A magnetic nanoprobe technology for detecting molecular interactions in live cells. Science 309, 121 (2005).
11. Wender, P. A. et. al. The design, synthesis and evaluation of molecules that enable or enhance cellular uptake: peptoid molecular transporters. Proc. Natl. Acad. Sci. USA 97, 13003 (2000).
12. Futaki, S., et al. Arginine-rich Peptides. J. Biol. Chem., 276(8), 5836-5840 (2001).
13. Somiari S, Glasspool-Malone J, Drabick J J, Gilbert R A, Heller R, Jaroszeski M J, Malone R W. Theory and in vivo application of electroporative gene delivery. Mol Ther. 2(3):178-87 (2002).
14. Nikoloff, Jac A., ed. Animal Cell Electroporation and Electrofusion Protocols, Methods in Molecular Biology, Volume 48, Humana Press, Totowa, N.J. (1995).
15. Scherer, F., et al., Magnetofection: enhancing and targeting gene delivery by magnetic force in vitro and in vivo. Gene Ther. 9, 102-109 (2002).
16. Binz, H. K., Amstutz, P. and Plückthun, A. Engineering novel binding proteins from nonimmunoglobulin domains. Nat. Biotech. 23, 1257 (2005).
17. De Crescenzo, G. D., Litowsky, J. R., Hodges, R. S. and O'Connor-McCourt, M. D. Real-time monitoring of the interactions of two-stranded de novo designed coiled-coils: effect of chain length on the kinetic and thermodynamic constants of binding. Biochemistry 42, 1754 (2003).
18. Deyev, S. M., Waibel, R., Lebedenko, E. N., Schubiger, A. P. and Plückthun, A. Design of multivalent complexes using the barnase-barstar module. Nat. Biotechnol. 21, 1486 (2003).
19. Link, S. and El-Sayed, M. A., Optical Properties and Ultrafast Dynamics of Metallic Nanocrystals. Annu. Rev. Phys. Chem. 54, 331-66 (2003).
20. Blasse, G. and Grabmeier, B. C., Luminescent Materials; Springer-Verlag: Berlin (1994).
21. Beaureparie, E., et al., Functionalized Fluorescent Oxide Nanoparticles: Artificial Toxins for Sodium Channel Targeting and Imaging at the Single-Molecule Level, Nano Letters 4, 2079 (2004).
22. Kelly, K. L., Coronado, E., Zhao, L. L. and Schatz, G. C. The optical properties of metal nanoparticles: the influence of size, shape and dielectric environment. J. Phys. Chem. B. 107, 668 (2003).
23. Schultz, S., Smith, D. R., Mock, J. J. and Schultz, D. A. Single-target molecule detection with nonbleaching multicolor optical immunolables. Proc. Natl. Acad. Sci. USA 97, 996 (2000).
24. Su, K. H. et. al. Interparticle coupling effects on plasmon resonances of nanogold particles. Nano Letters 3, 1087 (2003).
25. Rechberger, W. et. al. Optical properties of two interacting gold nanoparticles. Opt. Comm. 220, 137 (2003).
26. Prikulis, J. et. al. Optical spectroscopy of single trapped metal nanoparticles in solution. Nano Letters 4, 115 (2004).
27. Dudev, T., Lin, Y. L., Dudev, M. and Lim, C. First-second shell interactions in metal binding sites in proteins: a PDB survey and DFT/CDM calculations. J. Am. Chem. Soc. 125, 3168 (2003).
28. Levy, R., Thanh, N. T., Doty R. C., Hussain, I., Nichols, R. J., Schiffrin, D. J., Brust, M. and Fernig, D. G. Rational and combinatorial design of peptide capping ligands for gold nanoparticles. J Am Chem Soc. 126(32):10076-84 (2004).
29. Pederson, C. J. J. Am. Chem. Soc. 89, 2498, 7017 (1967).
30. Gokel G W, Leevy W M, Weber M E., Crown ethers: sensors for ions and molecular scaffolds for materials and biological models. Chem Rev. 104(5):2723-50 (2004).
31. Cram, D. J.; Cram, Accts. Chem. Res. 11, 8 (1978).
32. Lin, S-Y., et al., Recognition of Potassium Ion in Water by 15-Crown-5 Functionalized Gold Nanoparticles. Anal. Chem. 74, 330-335 (2002).
33. Masala, O. and Seshadri, R. Synthesis Routes for Large Volumes of Nanoparticles. Annu. Rev. Mater. Res. 34, 41-81 (2004).
34. Jares-Erijman, E. A. and Jovin, T. M. FRET imaging. Nat. Biotech. 21, 1387 (2003).
35. Bagwe, R. P., Yang, C., Hilliard, L. R. and Tan, W. Optimization of dye-doped silica nanoparticles prepared using a reverse microemulsion method. Langmuir 20, 8336 (2004).
36. Worley, J. F. and Main, M. J., An Industrial Perspective on Utilizing Functional Ion Channel Assays for High Throughput Screening. Receptors and Channels, 8, 269-282 (2002).
37. Jiang, Y., Ruta, V., Chen, J., Lee, A. and MacKinnon, R. The principle of gating charge movement in a voltage-dependent K⁺ channel. Nature 423, 42 (2003).
38. Huang, Z., Structural chemistry and therapeutic intervention of protein-protein interactions in immune response, human deficiency virus entry, and apoptosis. Pharm. & Ther. 86: 201-215 (2000).
39. Sun, Y. and Xia, Y. Increased sensitivity of surface plasmon resonance of gold nanoshells compared to that of gold solid colloids in response to environmental changes. Anal. Chem. 74, 5297 (2002).
40. Huber, K. Calcium-induced shrinking of polyacrylate chains in aqueous solution. J. Phys. Chem. 97, 9825 (1993).
41. Gittins, D. I. and Caruso, F. Tailoring the polyelectrolyte coating of metal nanoparticles. J. Phys. Chem. B 105, 6846 (2001).
42. Mossakowska, D. E., et al., Biochemistry, 28, 3843-3850 (1989).
43. Stober, W., Fink, A. and Bohn, E. J. Colloid Interface Sci. 26, 62 (1968).
44. Van Blaaderen, A. and Vrij, A. Synthesis and characterization of colloidal dispersions of fluorescent, monodisperse silica spheres. Langmuir 8, 2921 (1992).
45. Lai, C-Y, Trewyn, B. G., Jeftinija, D. M., Jeftinija, K., Xu, S., Jeftinija, S. and Lin, V. S-Y. A mesoporous silica nanosphere-based carrier system with chemically removable CdS nanoparticle caps for stimuli-responsive controlled release of neurotransmitters and drug molecules. J. Am. Chem. Soc. 125, 4451 (2003).
46. Lin, Y., et al., Well-ordered mesoporous silica nanoparticles as cell markers, J. Am. Chem. Soc., 17, 4570-4573 (2005).
47. Murphy, M. B., et al., Nucleic Acids Res., 31(18) (2003).
48. Alber, T. Curr. Opin. Genet. Devel. 2, 205 (1992).
49. Mason, J. M. and Arndt., K. M., Chem Bio Chem, 5:170-176 (2004).
50. Burkhard, P., Stetefeld, J., and Strelkov, S. V. Coiled-coils: A highly versatile protein folding motif. Trends Cell Biol. 11: 82-88 (2001).
51. Burkhard, P., Ivaninskii, S., and Lustig, A. Improving coiled-coil stability by optimizing ionic interactions. J. Mol. Biol. 318: 901-910 (2002).
52. Arndt, K., et al., A heterodimeric coiled-coil peptide pair selected in vivo from a designed library-versus-library ensemble. J Mol. Biol., 295(3):627-39 (2000).
53. Walshaw, J., and Woolfson, D N., J Mol. Biol. 307(5):1427-50 (2001).
54. Strelkov, S V. and Burkhard, P., J Struct Biol, 137(1-2):54-64 (2002).
55. van Gent, D., et al., Int J Biochem Cell Biol., 35(11):1536-47 (2003).
56. Shapiro R., Cytoplasmic ribonuclease inhibitor. Methods Enzymol., 341, 611-628 (2001).
57. Benhar, I., Biotechnol Adv., Biotechnological applications of phage and cell display. 19(1):1-33 (2001).
58. Binz, H K, et al., High-affinity binders selected from designed ankyrin repeat protein libraries. Nat Biotechnol., 22(5):575-82 (2004).
59. Wahlberg, E., et al., Proc Natl Acad Sci U S A, 100(6):3185-90 (2003).
60. Schlehuber, S. and Skerra A., Biophys Chem., 96(2-3):213-28 (2002).
61. Masala, O., and Seshadri, R., Synthesis routes for large volumes of nanoparticles. Ann. Rev. Mater. Res. Vol. 34: 41-81 (2004).
62. Sun, Y. and Xia, Y., Shape-Controlled Synthesis of Gold and Silver Nanoparticles, Science, 298: 2176-2179 (2002).
63. Hao E, et al., J Fluoresc. 14(4):331-41(2004).
65. Gil; l, I. and Ballesteros, A., Bioencapsulation within synthetic polymers (Part 1): sol-gel encapsulated biologicals, Tibtech, Vol. 18 (2002).
66. Li, Y., and Yip. W. T., Liposomes as Protective Capsules for Active Silica Sol-Gel Biocomposite Synthesis, J. Am. Chem. Soc., 127: 12756-12757 (2005).
67. Gill, I., Bio-doped Nanocomposite Polymers: Sol-Gel Bioencapsulates, Chem. Mater., 13:3403-3421 (2001).
68. Xu, Y., et al. Proc Natl Acad Sci USA, 96(1):151-6 (1999).
69. Qhobosheane, M., et al., Analyst, 126, 1274-78 (2001).
70. Zhao, X., et al., Adv. Mat., 16(2), 173-176 (2004).
71. Wang, L., et al., Nano Letters, Vol. 5(1), 37-43 (2005).
72. Ow, H., et al., Nano Letters, Vol. 5(1), 113-117 (2005).
73. Perez, J. M., et al., Nat. Biotechnol., 20, 816-820 (2002).
74. Chan, W. C W. et al, Luminescent quantum dots for multiplexed biological detection and imaging. Curr. Op. in Biotech., 13, 40-46 (2002).
75. Zheng, J, et al., Highly Fluorescent, Water-Soluble, Size-Tunable Gold Quantum Dots, Phys. Rev. Lett., 93(7) (2004).

76. Zheng, J, et al., Highly Fluorescent, Water-Soluble, Size-Tunable Gold Quantum Dots, Phys. Rev. Lett., 93(7), August 2004.
77. Takahashi, A., et al., Measurement of Intracellular Calcium, Pharmacological Reviews, 79(4), 1089-1125 (1999).
78. Lindhout, D. A., Litowski, J. R., Mercier, P., Hodges, R. S. and Sykes, B. D. NMR solution structure of a highly stable de novo heterodimeric coiled-coil. Biopolymers 75, 367 (2004).
79. O'Shea, E. K., Lumb, K. J. and Kim, P. S. Curr. Biol. 3, 658 (1993).
80. Glover, J. N. and Harrison, S. C. Nature 373, 257 (1995).
81. Pelletier, J. N., Arndt, K. M., Plückthun, A. and Michnick, S. W. An in vivo library-versus-library selection of optimized protein-protein interactions. Nat. Biotech. 17, 683 (1999).
82. Mock, J. J., Smith, D. R. and Schultz, S. Local refractive index dependence of plasmon resonance spectra from individual nanoparticles. Nano Letters 3, 485 (2003).
83. McFarland, A. D. and Van Duyne, R. P. Single silver nanoparticles as real-time optical sensors with zeptomole sensitivity. Nano Letters 3, 1057 (2003).
84. Clapham, D., TRP channels as cellular sensors. Nature, 426, 517 (2003).
85. Baubet, V., et al., Chimeric green fluorescent protein-aequorin as bioluminescent Ca2+ reporters at the single-cell level, Proc. Natl. Acad. Sci. 97(13), 7260-7265 (2000).
86. Bastiaens, P A and Squire, A., Fluorescence lifetime imaging microscopy: spatial resolution of biochemical processes in the cell Trends in Cell Biol. 9:48-52 (1999).
87. Jares-Erijman E A, Jovin T M, FRET imaging, Nat Biotechnol. 21(11):1387-95 (2003)
88. Herman B, Krishnan R V, Centonze V E, Microscopic analysis of fluorescence resonance energy transfer (FRET), Methods Mol. Biol., 261:351-70 (2004).
89. Kim, J., et al., Ion-Specific Aggregation in Conjugated Polymers: Highly Sensitive and Selective Fluorescent Ion Chemosensors. Angew. Chem. Int. Ed., 39, 3868-3872 (2000).
90. Bockaret, J., et al., G protein-coupled receptors: dominant players in cell-cell communication. Int. Rev. Cytol. 212, 63-132 (2002).
91. Cabrera-Vera, T. M., et al., Insights into G protein strucgture, function, and regulation. Endocr. Rev., 24, 765-781 (2003).
92. Dolphin, A. C., β-subunits of Voltage-Gated Calcium Currents, J. Bioenerg. Biomemb., 35(6). 599-620 (2003).
93. Kim, Y., et al., Nano Letters, 1, 165 (2001).
94. Bastiaens, P A and Squire, A., Fluorescence lifetime imaging microscopy: spatial resolution of biochemical processes in the cell. Trends in Cell Biol. 9:48-52 (1999).
95. Nakai, J., Ohkura, M. and Imoto, K. A high signal-to-noise Ca⁺⁺ probe composed of a single green fluorescent protein. Nat. Biotechnol. 19, 137 (2001).
96. Rudolf, R., Mongillo, M., Rizzuto, R. and Pozzan, T. Looking forward to seeing calcium. Nat. Rev. Mol. Cell. Bio. 4, 579 (2003).

We claim:

1. A nanosensor component comprising:
a nanoparticle having a moiety comprising a recognition domain attached to the nanoparticle, wherein the recognition domain is selected to specifically bind to a target domain of a cellular polypeptide, and wherein:
(i) the recognition domain comprises a coiled-coil peptide, an enzyme, or an enzyme inhibitor; or
(ii) the nanoparticle comprises an ion-sensitive fluorescent or luminescent moiety or comprises a resonance energy transfer (RET) donor having an emission spectrum capable of exciting an ion-sensitive RET donor; or
(iii) the nanoparticle comprises a coating layer comprising a material that undergoes a change in refractive index in the presence of a ligand; or
(iv) any combination of (i)-(iii).

2. The nanosensor component of claim 1, wherein the cellular polypeptide is a recombinant polypeptide comprising a heterologous target domain.

3. The nanosensor component of claim 2, wherein the cellular polypeptide is an ion channel subunit.

4. The nanosensor component of claim 2, wherein the nanoparticle is a metal nanoparticle.

5. The nanosensor component of claim 2, wherein the nanoparticle is a plasmon resonant metal particle.

6. (canceled)

7. The nanosensor component of claim 2, wherein the nanoparticle comprises a quantum dot.

8. (canceled)

9. The nanosensor component of claim 2, wherein the nanoparticle is made at least in part of silica.

10. The nanosensor component of claim 9, wherein the nanoparticle is made at least in part of mesoporous silica.

11. The nanosensor component of claim 2, wherein the recognition domain comprises a coiled-coil peptide.

12. The nanosensor component of claim 2, wherein the recognition domain comprises an enzyme or enzyme inhibitor.

13. (canceled)

14. The nanosensor component of claim 2, comprising a ligand-responsive binding moiety present at the surface of the nanoparticle.

15. The nanosensor component of claim 14, wherein the binding moiety is responsive to an ion.

16. The nanosensor component of claim 14, wherein the binding moiety is responsive to calcium ions.

17. (canceled)

18. The nanosensor component of claim 14, wherein presence of the ligand causes association of multiple binding moieties with one another.

19. The nanosensor component of claim 2, further comprising a delivery moiety attached to the nanosensor component.

20. The nanosensor component of claim 2, wherein the delivery moiety comprises a peptide.

21. (canceled)

22. (canceled)

23. (canceled)

24. (canceled)

25. (canceled)

26. (canceled)

27. (canceled)

28. (canceled)

29. A cell comprising the nanosensor component of claim 2.

30. A polypeptide comprising a recognition domain selected from the group consisting of: heterologous coiled-coil domains, enzymes, and enzyme inhibitors, wherein the polypeptide is selected from the group consisting of ion channel subunits, G protein subunits, and G protein coupled receptors (GPCRs).

31. The polypeptide of claim 30, wherein the recognition domain is a heterologous coiled-coil domain and the polypeptide is an ion channel subunit.

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. A method of detecting ion channel or GPCR activity or lack thereof comprising steps of:
(i) providing a cell comprising a polypeptide having a nanosensor component attached thereto, wherein the polypeptide is an ion channel subunit or GPCR, and wherein the nanosensor component comprises a nanoparticle;
(ii) maintaining the cell under conditions in which ion channel or GPCR activity may occur; and
(iii) detecting a signal that is indicative of ion channel or GPCR activity or lack thereof.

58. The method of claim 57, wherein the signal is an optical or magnetic property of a nanoscale sensor comprising the nanosensor component attached to the polypeptide.

59. The method of claim 57, wherein detecting the signal comprises detecting a spectral feature of an absorption, extinction, or scattering spectrum.

60. The method of claim 57, wherein detecting the signal comprises detecting a peak wavelength of an absorption, extinction, or scattering spectrum.

61. The method of claim 57, wherein detecting the signal comprises detecting a shift in peak wavelength of an absorption, extinction, or scattering spectrum relative to the peak wavelength that would exist in the absence of ion channel activity.

62. The method of claim 57, wherein the signal is a fluorescent or luminescent signal.

63. The method of claim 57, wherein the signal is proximity-dependent.

64. The method of claim 57, wherein the nanoparticle is a magnetic nanoparticle and detecting the signal comprises detecting a relaxation time of water or detecting a change in a relaxation time of water.

65. The method of claim 57, wherein detecting the signal comprises obtaining an image of the cell.

66. (canceled)

67. (canceled)

68. The method of claim 57, wherein the nanoparticle comprises an ion-sensitive fluorescent or bioluminescent molecule.

69. The method of claim 57, wherein the nanoparticle comprises a RET donor that has an emission spectrum capable of exciting an ion-sensitive RET acceptor.

70. (canceled)

71. (canceled)

72. The method of claim 57, wherein the nanoparticle comprises a delivery moiety.

73. (canceled)

74. The method of claim 57, wherein the nanoparticle is made at least in part of silica.

75. The method of claim 57, wherein the nanoparticle is made at least in part of mesoporous silica.

76. The method of claim 57, wherein the polypeptide is an ion channel subunit having at two ligand-responsive nanoparticles attached to it, or wherein the polypeptide is a subunit of an ion channel that comprises at least two subunits each having a nanoparticle sensor component attached thereto.

77. The method of claim 76, wherein passage of a ligand through the ion channel upon channel activity causes reversible or irreversible nanoparticle aggregation.

78. The method of claim 57, wherein the ion channel has at least one nanosensor component comprising a ligand-responsive nanoparticle attached to a subunit.

79. The method of claim 78, wherein passage of a ligand through the ion channel upon channel activity causes a change in refractive index in the local environment of the nanoparticle.

80. (canceled)

81. (canceled)

82. The method of claim 57, wherein the polypeptide comprises a heterologous target domain and the nanosensor component comprises a recognition domain that binds to the target domain.

83. The method of claim 82, wherein the recognition domain and the target domain are selected from the group consisting of: coiled-coil peptides, enzymes, and enzyme inhibitors.

84. (canceled)

85. The method of claim 57, further comprising the step of:
contacting the cell with a compound.

86. The method of claim 85, further comprising the step of:
identifying the compound as an ion channel or GPCR modulator if the activity of the ion channel or GPCR, respectively, differs from that which would be exhibited by a control cell.

87. A method of testing a compound comprising steps of:
(i) providing a cell comprising an ion channel or GPCR having a nanosensor component attached thereto, wherein the nanosensor component comprises a nanoparticle;
(ii) contacting the cell with the compound;
(iii) gathering a signal indicative of ion channel or GPCR activity or lack thereof; and
(iv) determining whether the compound is a modulator of ion channel or GPCR activity based on the information gathered in step (iii).

88. The method of claim 87, wherein a population of cells is provided.

89. The method of claim 87, further comprising the step of:
contacting the cell with a known modulator of ion channel or GPCR activity.

90. A method of screening a compound library comprising steps of:
(i) providing a compound library; and
(ii) performing the method of claim 87 using a plurality of compounds from the library, each of which is contacted with a cell or population of cells.

91. (canceled)

92. (canceled)

93. (canceled)

94. (canceled)

95. (canceled)

96. (canceled)

97. (canceled)

98. (canceled)

99. A method of attaching a nanoparticle to a cellular polypeptide of interest comprising steps of:
(i) providing a cell that expresses a polypeptide of interest comprising a target domain;
(ii) contacting the cell with a nanoparticle comprising a recognition domain that corresponds to the target domain under conditions in which internalization of the nanoparticle occurs;
(iii) maintaining the cell so that the recognition domain and the target domain bind to one another within the cell.

100. The method of claim 99, wherein the target domain is a heterologous target domain.

101. (canceled)

102. (canceled)

103. (canceled)

104. (canceled)

105. (canceled)

106. (canceled)

107. (canceled)

108. (canceled)

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. (canceled)

131. (canceled)

132. (canceled)

133. (canceled)

134. (canceled)

135. (canceled)

136. (canceled)

137. (canceled)

138. (canceled)

139. (canceled)

140. (canceled)

141. (canceled)

142. (canceled)

143. (canceled)

144. (canceled)

145. (canceled)

146. (canceled)

147. (canceled)

148. (canceled)

149. A method of detecting ion channel activity comprising steps of:
(i) providing a cell comprising at least one ion channel having a nanoparticle attached thereto, wherein the nanoparticle comprises an ion-sensitive signal-generating moiety; and
(ii) detecting a signal indicative of ion channel activity.

150. The method of claim 149, wherein the signal-generating moiety is an ion-sensitive fluorescent or luminescent molecule.

151. A method of detecting ion channel activity comprising steps of:
(i) providing a cell comprising at least one ion channel having a nanoparticle attached thereto, wherein the nanoparticle comprises a RET donor and the cell contains an ion-sensitive RET acceptor, wherein the RET donor and RET acceptor are a RET pair; and
(ii) detecting RET between the donor and acceptor, wherein RET is indicative of ion channel activity.

152. (canceled)

153. (canceled)

154. (canceled)

155. (canceled)

156. A method of detecting ion channel activity comprising steps of:
(i) providing a cell comprising at least one ion channel having at least two nanoparticles attached thereto; and
(ii) detecting an alteration in the distance between the nanoparticles, wherein the alteration in distance is indicative of ion channel activity.

157. (canceled)

158. (canceled)

159. (canceled)

160. (canceled)

161. (canceled)

162. (canceled)

163. (canceled)

164. (canceled)

165. (canceled)

166. (canceled)

167. (canceled)

168. (canceled)

169. (canceled)

170. (canceled)

171. (canceled)

172. (canceled)

173. (canceled)

174. (canceled)

175. (canceled)


SEQUENCE LISTING (46 sequences)

SEQ ID NO: 1 - protein, 7 aa, Artificial
  Target domain
KVSALKE

SEQ ID NO: 2 - protein, 7 aa, Artificial
  Target domain
EVSALEK

SEQ ID NO: 3 - protein, 21 aa, Artificial
  Recognition domain
KVSALKEKVSALKEKVSALKE

SEQ ID NO: 4 - protein, 28 aa, Artificial
  Recognition domain
EVSALEKEVSALEKEVSALEKEVSALEK

SEQ ID NO: 5 - protein, 21 aa, Artificial
  Complementary peptides
EIAALEKEIAALEKEIAALEK

SEQ ID NO: 6 - protein, 21 aa, Artificial
  Complementary peptides
KIAALKEKIAALKEKIAALKE

SEQ ID NO: 7 - protein, 30 aa, Artificial
  Coiled-coil peptides
AQLEKEKLALEQENAQLEWELQALEKELAQ

SEQ ID NO: 8 - protein, 30 aa, Artificial
  Coiled-coil peptides
AQLKKKLQALKKKNAQLKWKLQALKKKLAQ

SEQ ID NO: 9 - protein, 32 aa, Artificial
  Coiled coil peptides
IARLEEKVKTLKAQNSELASTANMLREQVAQL

SEQ ID NO: 10 - protein, 32 aa, Artificial
  Coiled-coil peptides
TDTLQAETDQLEDEKSALQTEIANLLKEKEKL

SEQ ID NO: 11 - protein, 32 aa, Artificial
  Target domains based on the Winzip-A2B1 heterodimer.
VAQLRERVKTLRAQNYELESEVQRLREQVAQL

SEQ ID NO: 12 - protein, 32 aa, Artificial
  Target domains based on the Winzip-A2B1 heterodimer.
VDELQAEVDQLQDENYALKTKVAQLRKKVEKL

SEQ ID NO: 13 - protein, 5 aa, Artificial
  Suitable peptides
CALNN

SEQ ID NO: 14 - protein, 5 aa, Artificial
  Suitable peptides
CTTTT

SEQ ID NO: 15 - protein, 6 aa, Artificial
  Suitable peptides
CCALNN

SEQ ID NO: 16 - protein, 6 aa, Artificial
  Suitable peptides
CCVVVT

SEQ ID NO: 17 - protein, 7 aa, Artificial
  Peptides having a terminal lysine residue
CALNNGK

SEQ ID NO: 18 - protein, 9 aa, Artificial
  TAT peptide
RKKRRQRRR

SEQ ID NO: 19 - protein, 12 aa, Artificial
  TAT peptide
YGRKKRRQRRRP

SEQ ID NO: 20 - protein, 13 aa, Artificial
  TAT peptide
GRKKRRQRRRPPQ

SEQ ID NO: 21 - protein, 16 aa, Artificial
  Antennapedia protein
RQIKIWFZQRRMKWKK

SEQ ID NO: 22 - protein, 28 aa, Artificial
  Transportan - 27
GWYLNSAGYLLGKCINLKALAALAKKIL

SEQ ID NO: 23 - protein, 23 aa, Artificial
  Transportan - 22
GWYLNSAGYLLGKCINLKALAAL

SEQ ID NO: 24 - protein, 13 aa, Artificial
  TAT peptide
GLFEALEELWEAK

SEQ ID NO: 25 - protein, 11 aa, Artificial
  TAT peptide
CGGRKKRRRRR

SEQ ID NO: 26 - DNA, 60 bp, Artificial
  Forward primer
cccgctagcgccaccatgaaggtaagcgccttcaaggagaaagtgtccgcactgaaagaa

SEQ ID NO: 27 - DNA, 59 bp, Artificial
  Forward primer
aagtgtccgcactgaaagaaaaagtttctgcactgaaggaaaaggtctcggcactcaag

SEQ ID NO: 28 - DNA, 49 bp, Artificial
  Forward primer
aaggtctcggcactcaaggaaggtggtggcatggtgagcaagggcgagg

SEQ ID NO: 29 - DNA, 18 bp, Artificial
  Reverse primer
ggttcagggggaggtgtg

SEQ ID NO: 30 - DNA, 39 bp, Artificial
  PCR reaction A primer
gatccgctagccaccatgaaaaaagcagtcattaacggg

SEQ ID NO: 31 - DNA, 48 bp, Artificial
  PCT reaction A primer
cagctcctcgcccttgctcaccatagaaagtatgatggtgatgtcgca

SEQ ID NO: 32 - DNA, 48 bp, Artificial
  PCR reaction B primer
tgcgacatcaccatcatactttctatggtgagcaagggcgaggagctg

SEQ ID NO: 33 - DNA, 18 bp, Artificial
  PCR reaction B primer
ggttcagggggaggtgtg

SEQ ID NO: 34 - DNA, 30 bp, Artificial
  Mouse
aggcaagcttcgatgaatgcccactccaag

SEQ ID NO: 35 - DNA, 28 bp, Artificial
  Mouse
aattggatccctacaccgacgtttctgg

SEQ ID NO: 36 - DNA, 29 bp, Artificial
  Rat TRPV1 cDNA
gatgtcgacagatggaacaacgggctagc

SEQ ID NO: 37 - DNA, 28 bp, Artificial
  Rat TRPV1 cDNA
tgcagaattcttatttctcccctgggac

SEQ ID NO: 38 - DNA, 33 bp, Artificial
  Rat P2X2 cDNA
aattagatctaccatggtccggcgcttggcccg

SEQ ID NO: 39 - DNA, 31 bp, Artificial
  Rat P2X2 cDNA
tttgtcgactcaaagttgggccaaacctttg

SEQ ID NO: 40 - DNA, 40 bp, Artificial
  Sequence encoding for barnase with BamHI and XhoI
aaaggatccgccaccatggcacaggttatcaacacgtttg

SEQ ID NO: 41 - DNA, 40 bp, Artificial
  Sequence encoding for barnase with BamHI and XhoI
ttccctcgagtcagcaacagtgatggtgatgatggtgcgg

SEQ ID NO: 42 - DNA, 33 bp, Artificial
  Haemaglutinin-Barnase and 6X-GST-Barnase constructs
tacaaaacaacggacgcttatcagacctttaca

SEQ ID NO: 43 - DNA, 33 bp, Artificial
  Haemaglutinin-Barnase and 6X-GST-Barnase constructs
tgtaaaggtctgataagcgtccgttgttttgta

SEQ ID NO: 44 - protein, 21 aa, Artificial
  Domain peptide
KVSALKEKVSALKEKVSALKE

SEQ ID NO: 45 - protein, 28 aa, Artificial
  Target domains that interact to form a coiled-coil
EVSALEKEVSALEKEVSALEKEVSALEK

SEQ ID NO: 46 - protein, 24 aa, Artificial
  Is a schematic representation showing a recognition design based on the barnase-barstar complex.
CGGKVSALKEKVSALKEKVSALKE